(19) United States
(12) Patent Application Publication
(10) Pub. No.: US 2022/0177466 A1
(43) Pub. Date: Jun. 9, 2022

(54) DEGRADERS OF KELCH-LIKE ECH-ASSOCIATED PROTEIN 1 (KEAP1)

(71) Applicant: DANA-FARBER CANCER INSTITUTE, INC., Boston, MA (US)

(72) Inventors: Nathanael Gray, Stanford, CA (US); Tinghu Zhang, Brookline, MA (US); Guangyan Du, Roslindale, MA (US); Nathaniel Henning, Brookline, MA (US); Jie Jiang, Brookline, MA (US); Eric Fischer, Chestnut Hill, MA (US); Katherine Donovan, Boston, MA (US)

(73) Assignee: DANA-FARBER CANCER INSTITUTE, INC., Boston, MA (US)

(21) Appl. No.: 17/601,536

(22) PCT Filed: Apr. 7, 2020
(86) PCT No.: PCT/US2020/027067

§ 371 Date: Oct. 5, 2021

Related U.S. Application Data
(60) Provisional application No. 62/830,670, filed on Apr. 8, 2019.

(51) Int. Cl. C07D 419/04; C07D 401/14; C07D 401/04; A61K 47/54

(52) U.S. Cl. CPC C07D 419/04; A61K 47/545; C07D 401/04; C07D 401/14

(57) ABSTRACT

The present invention relates to bifunctional compounds, compositions, and methods for treating diseases or conditions mediated by Kelch-like ECH-associated protein 1 (KEAP1). In some aspects, the present invention is directed to methods of treating diseases or disorders involving dysfunctional (e.g., dysregulated) KEAP1 activity, that entails administration of a therapeutically effective amount of a bifunctional compound of formula I or a pharmaceutically acceptable salt or stereoisomer thereof, to a subject in need thereof.

### RELATED APPLICATIONS

This application claims the benefit of priority under 35 U.S.C. § 119(e) to U.S. Provisional Application No. 62/830,670, filed Apr. 8, 2019, which is incorporated herein by reference in its entirety.

### GOVERNMENT LICENSE RIGHTS

This invention was made with government support under grant number 1R01 CA214608 awarded by the National Institutes of Health. The government has certain rights in the invention.

### BACKGROUND OF THE INVENTION

Immunomodulatory drugs (IMiDs) are a new class of anticancer compounds exemplified by thalidomide, lenalidomide, and pomalidomide. Thalidomide, lenalidomide, and pomalidomide are all FDA approved anti-myeloma drugs. The common primary cellular target is cereblon (CRBN), an E3 ligase adapter protein. These IMiD compounds induce degradation of the transcription factors Ikaros family of zinc-finger proteins 1 and 3 (IKZF1/3) and lead to apoptosis in cancer cell. Beyond that, accumulating evidence has shown that these IMiDs compounds can be further developed as “cereblon modulators” which can degrade different protein targets. For example, CC-885, an analog of thalidomide has been shown to induce degradation of the translation termination factor Gi to S phase transition 1 (GSPT1), resulting in an antiproliferative effect in acute myeloid leukemia. Structural analyses have revealed that CC-885 provides an interaction hotspot between CRBN and GSPT1. On the other hand, there are extensive developments on the linker-based approach frequently called “degraders” or “proteolysis targeting chimera (PROTACs)” for targeted protein degradation via CRBN. Several proteins, such as bromodomain containing 4 (BRD4) and cyclin dependent kinase 9 (CDK9) have been successfully degraded by well-designed PROTAC compounds.

Kelch-like ECH-associated protein 1 (KEAP1) is the key regulator of the nuclear factor-erythroid 2 p45-related factor 2 (NRF2)-mediated cytoprotective response. Under basal conditions, NRF2 is sequestered by the dimeric KEAP1/Cullin3 (Cul3) complex, becoming ubiquitinated and degraded by the proteasome. An increase in levels of electrophilic species results in covalent modification of cysteine residues in the BTB and IVR domains, and leads to dissociation of Cul3 and other conformational changes (Schneekloth et al., Chembiochem. 6:40-46 (2005); Lu et al., Chem. Biol. 22:755-763 (2015)). This perturbs the ubiquitination of NRF2, allowing translocation to the nucleus, where it activates antioxidant response elements (AREs) to increase expression of cytoprotective proteins (Davies et al., J. Med. Chem. 59:3991-4006 (2016)). Inadequate nuclear factor erythroid 2 (NRF2) signaling in the face of chronic oxidative stress has been proposed as a pathological mechanism in inflammatory diseases; up-regulation of the pathway might be beneficial in a range of therapeutic areas, including cardiovascular, neurodegeneration, and respiratory (e.g., chronic obstructive pulmonary disease) conditions. KEAP 1 has become increasingly recognized as a target for diseases involving oxidative stress.

### SUMMARY OF THE INVENTION

A first aspect of the present invention is directed to a bifunctional compound which is represented by Formula I:

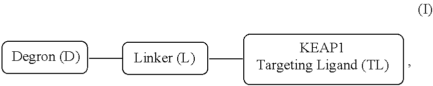

wherein the targeting ligand represents a moiety that binds KEAP1, the degron represents a moiety that binds an E3 ubiquitin ligase, and the linker represents a moiety that covalently connects the degron and the targeting ligand, or a pharmaceutically acceptable salt or stereoisomer thereof.

In another aspect, pharmaceutical compositions containing a therapeutically effective amount of the bifunctional compound of formula (I), or a pharmaceutically acceptable salt or stereoisomer thereof, and a pharmaceutically acceptable carrier are provided.

In yet another aspect of the present invention, methods of making the bifunctional compounds are provided.

A further aspect of the present invention is directed to a method of treating a disease or disorder mediated by dysfunctional (e.g., dysregulated) KEAP1 activity, that includes administering a therapeutically effective amount of the bifunctional compound of formula (I) or a pharmaceutically acceptable salt or stereoisomer thereof, to a subject in need thereof.

Without intending to be bound by any particular theory of operation, the bifunctional compounds of formula I are believed to promote the degradation of KEAP1 protein. By conjugating the KEAP1 binder-thalidomide compounds with a E3 ligase binder, these bifunctional degrader molecules are able to fast recruit E3 ligase, and therefore promote the degradation of KEAP1.

Accordingly, the bifunctional compounds of the present invention may serve as a set of new chemical tools for KEAP1 knockdown, exemplify a broadly applicable approach to arrive at degraders, and provide a potential treatment for many kinds of disease which are mediated by oxidative stress.

### BRIEF DESCRIPTION OF THE DRAWINGS

FIG. 1 is an immunoblot that shows the knockdown of KEAP1 in multiple myeloma (MM).1S cells after 15 hours at various concentrations for inventive compounds 1 and 4.

FIG. 2 is an immunoblot that shows the knockdown of KEAP1 in MM.1S cells over a period of 6 hours at 5 μM for inventive compounds 1 and 4.

FIG. 3 is a group immunoblots that show the knockdown of KEAP1 in MM.1S and MM.1S CRBN−/− cells over a period of 6 hours at 5 μM for inventive compounds 1 and 4.

FIG. 4A and FIG. 4B are immunoblots that show the knockdown of KEAP1 in MM.1S cells after 2 hours for inventive compounds 1 and 4, DGY-03-118, lenalidomide, bortezomib, and MLN4924.

FIG. 5A and FIG. 5B are immunoblots that show the knockdown of KEAP1 in MM.1S over a period of 24 hours at 5 μM for inventive compounds 1 and 4 with washout at 2, 4, 7, and 24 hours.

### DETAILED DESCRIPTION

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6]ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Examples of 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Examples of 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1 2 3, or 4) heteroatoms such as oxygen, sulfur, and nitrogen grouped in a linear, branched, or cyclic structural format.

Representative examples of substituents may thus include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

The term “binding” as it relates to interaction between the targeting ligand and the targeted protein or proteins, which in this invention is KEAP1, typically refers to an inter-molecular interaction that may be preferential or substantially specific in that binding of the targeting ligand with other proteinaceous entities present in the cell is functionally insignificant. The present bifunctional compounds bind and recruit KEAP1 for targeted degradation.

The term “binding”, as it relates to interaction between the degron and the E3 ubiquitin ligase, typically refers to an inter-molecular interaction that may or may not exhibit an affinity level that equals or exceeds that affinity between the targeting ligand and the target protein, but nonetheless wherein the affinity is sufficient to achieve recruitment of the ligase to the targeted degradation and the selective degradation of the targeted protein.

Broadly, the bifunctional compounds of the present invention have a structure represented by formula I:

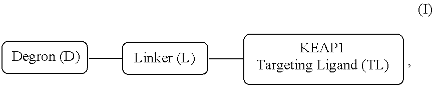

wherein the targeting ligand represents a moiety that binds Kelch-like ECH-associated protein 1 (KEAP1), the degron represents a moiety that binds an E3 ubiquitin ligase, and the linker represents a moiety that covalently connects the degron and the targeting ligand, or a pharmaceutically acceptable salt or stereoisomer thereof.

### KEAP1 Targeting Ligands

The KEAP1 targeting ligand (TL), which is a functional modality of the present compounds, binds KEAP1.

In some embodiments, the targeting ligand has a structure represented by formula TL-1a or TL-1b:

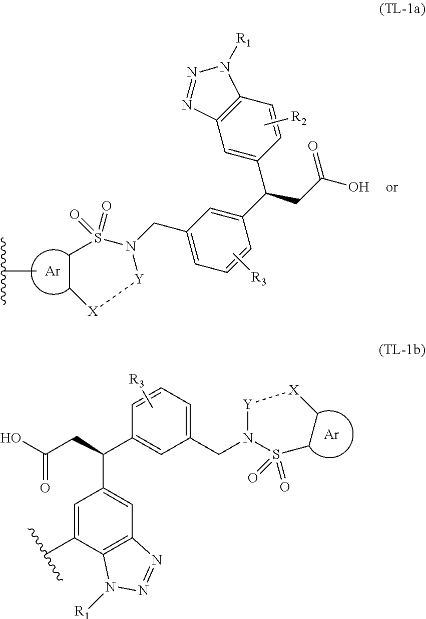

wherein:
R₁ represents optionally substituted C₁-C₃ alkyl;
R₂ represents Me, OMe, or halo;
R₃ represents Me, OMe, or halo;
X represents H and Y represents optionally substituted C₁-C₃ alkyl, or X represents —O—, Y represents optionally substituted —CH₂—CH₂— and X and Y together with the atoms to which they are bound form a 7-membered cyclic sulfonamide; and
Ar represents phenyl or pyridinyl.

Thus, in some embodiments, the bifunctional compounds of the present invention have a structure represented by formula I-1a or I-1b:

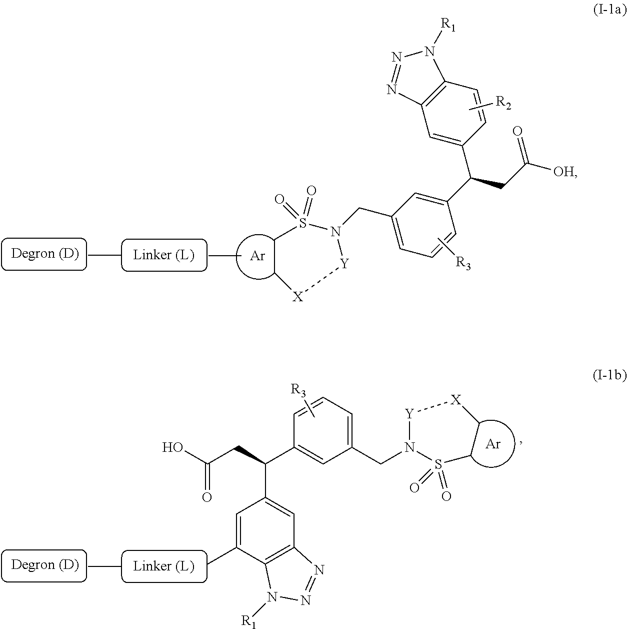

or a pharmaceutically acceptable salt or stereoisomer thereof;
wherein:
R₁ represents optionally substituted C₁-C₃ alkyl;
R₂ represents Me, OMe, or halo;
R₃ represents Me, OMe, or halo;
X represents H and Y represents optionally substituted C₁-C₃ alkyl, or X represents —O—, Y represents optionally substituted —CH₂—CH₂— and X and Y together with the atoms to which they are bound form a 7-membered cyclic sulfonamide; and
Ar represents phenyl or pyridinyl.

In certain embodiments, the targeting ligand has a structure represented by a structure selected from the group consisting of:

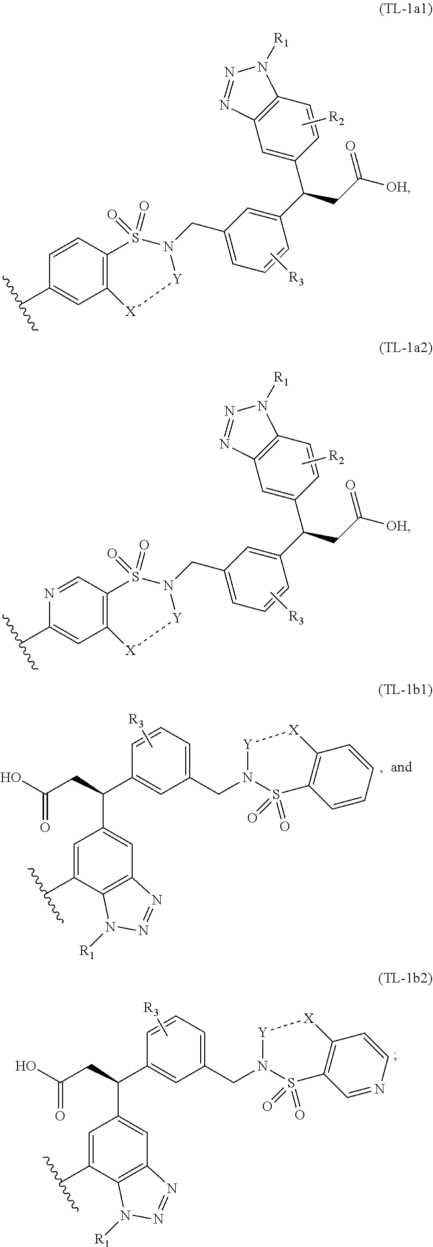

wherein:
R₁ represents optionally substituted C₁-C₃ alkyl;
R₂ represents Me, OMe, or halo;
R₃ represents Me, OMe, or halo;
X represents H and Y represents optionally substituted C1-C3 alkyl, or X represents —O—, Y represents optionally substituted —CH₂—CH₂— and X and Y together with the atoms to which they are bound form a 7-membered cyclic sulfonamide.

Thus, in certain embodiments, the bifunctional compounds of the present invention have a structure represented by a formula selected from the group consisting of:

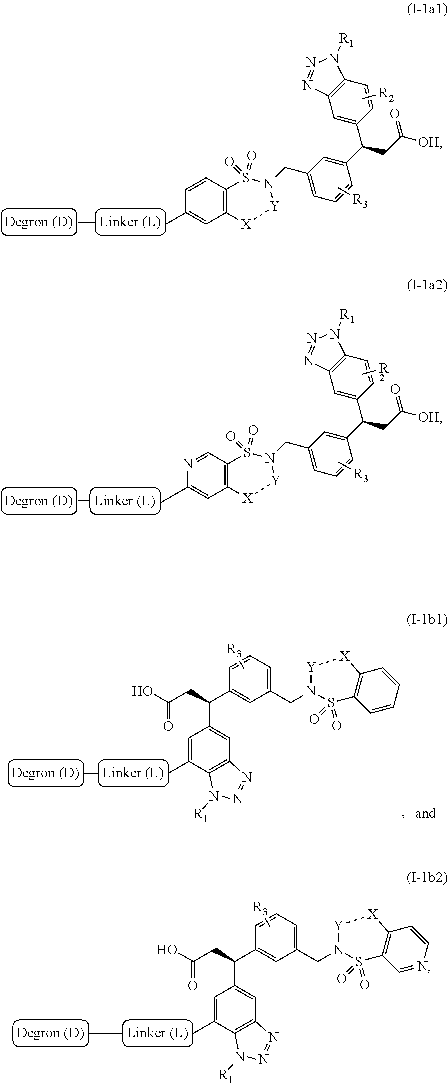

or a pharmaceutically acceptable salt or stereoisomer thereof;
wherein:
R₁ represents optionally substituted C1-C3 alkyl;
R₂ represents Me, OMe, or halo;
R₃ represents Me, OMe, or halo;
X represents H and Y represents optionally substituted C1-C3 alkyl, or X represents —O—, Y represents optionally substituted —CH₂—CH₂— and X and Y together with the atoms to which they are bound form a 7-membered cyclic sulfonamide.

In some embodiments, the targeting ligand has a structure represented by formula TL2:

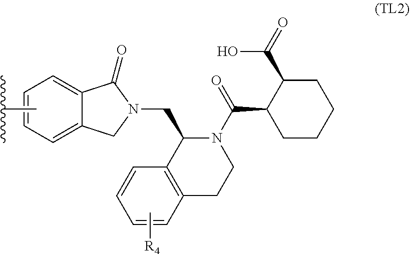

wherein:
R₄ represents optionally substituted C1-C3 alkyl, OMe, or halo.

Thus, in some embodiments, the bifunctional compounds of the present invention have a structure represented by formula I-2:

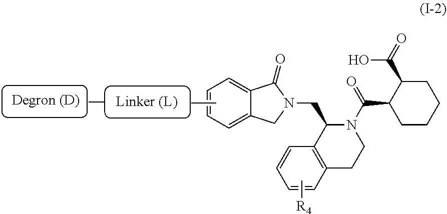

or a pharmaceutically acceptable salt or stereoisomer thereof;
wherein:
R₄ represents optionally substituted C1-C3 alkyl, OMe, or halo.

In some embodiments, the targeting ligand has a structure represented by formula TL3:

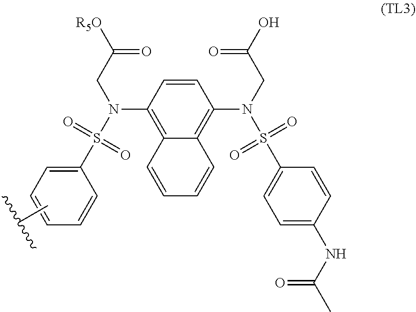

wherein:
R₅ represents optionally substituted C1-C3 alkyl.

Thus, in some embodiments, the bifunctional compounds of the present invention have a structure represented by formula (I-3):

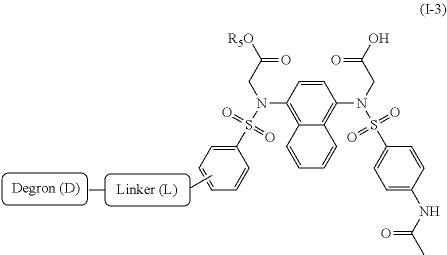

(I-3) or a pharmaceutically acceptable salt or stereoisomer thereof;
wherein:
R₅ represents optionally substituted C1-C3 alkyl.

In some embodiments, the bifunctional compounds of the present invention have a structure represented by a formula selected from the group consisting of:

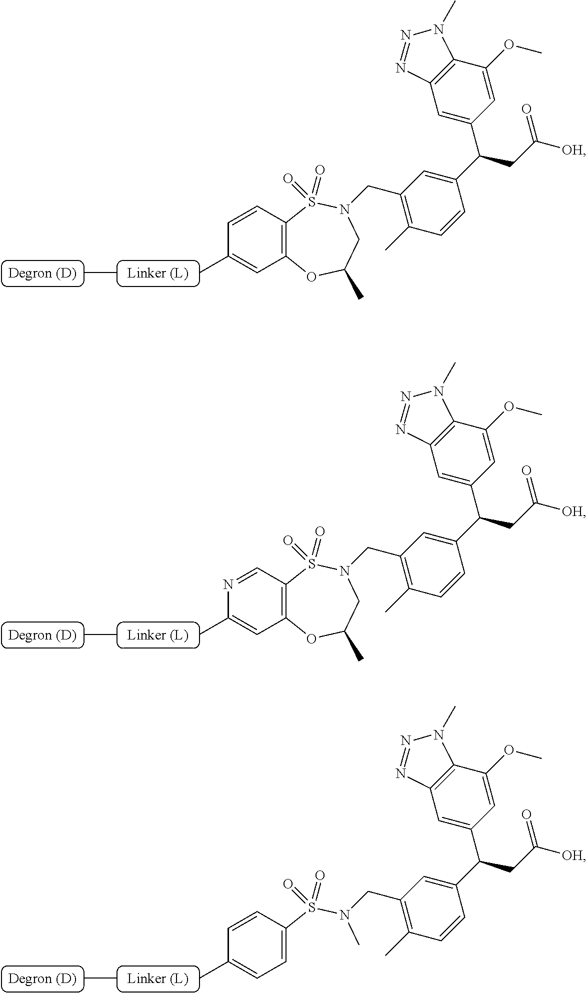

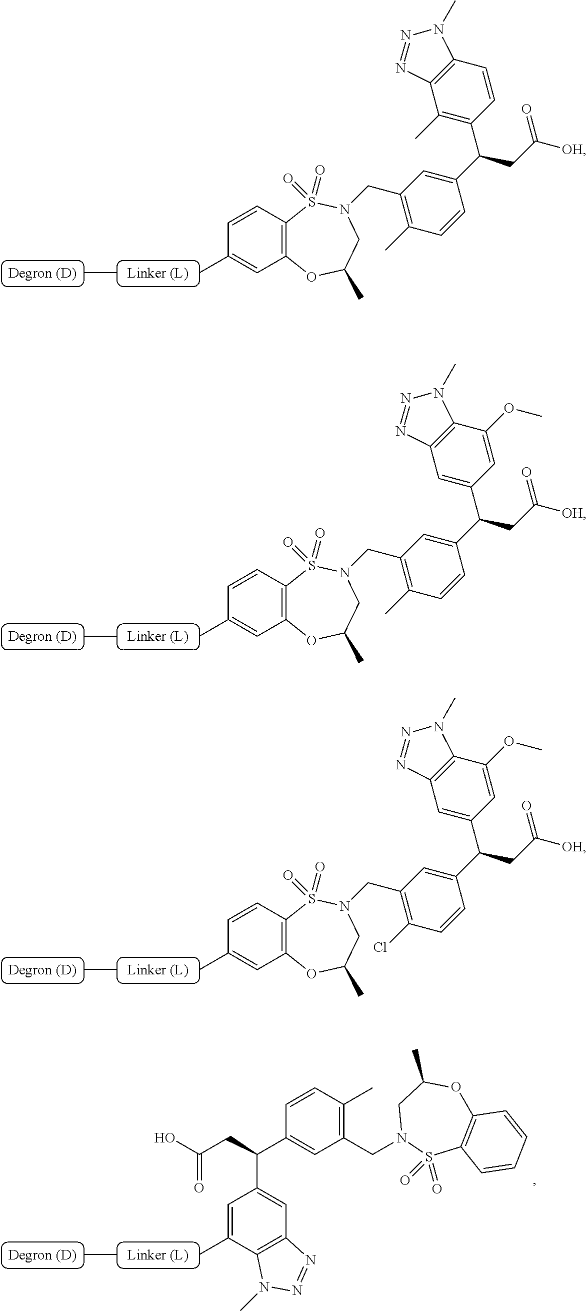

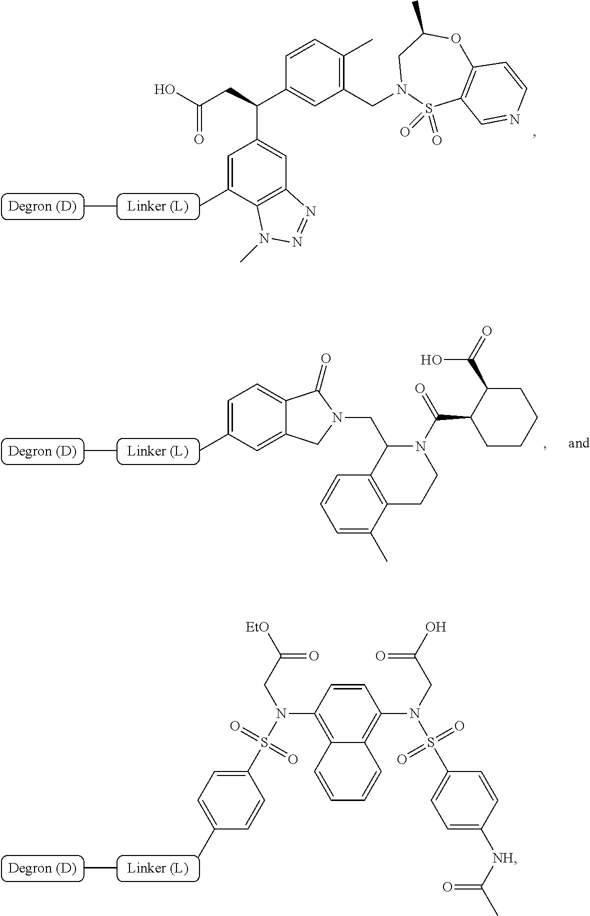

or a pharmaceutically acceptable salt or stereoisomer thereof.

### Linkers

The linker (“L”) provides a covalent attachment the targeting ligand and the degron. The structure of linker may not be critical, provided it does not substantially interfere with the activity of the targeting ligand or the degron. In some embodiments, the linker may be an alkylene chain or a bivalent alkylene chain, either of which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

In some embodiments, the linker may be a polyethylene glycol chain which may terminate (at either or both termini) in at least one of —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the one or both terminating groups may be the same or different.

“Carbocyclene” refers to a bivalent carbocycle radical, which is optionally substituted.

“Heterocyclene” refers to a bivalent heterocyclyl radical which may be optionally substituted.

“Heteroarylene” refers to a bivalent heteroaryl radical which may be optionally substituted.

In certain embodiments, the linker is an alkylene chain having 1-10 alkylene units and interrupted by or terminating in

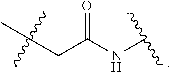 [SMILES: *CC(=O)N*]

In other embodiments, the linker is a polyethylene glycol chain having 2-8 PEG units and terminating in

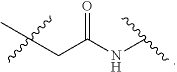 [SMILES: *CC(=O)N*]

Representative examples of linkers that may be suitable for use in the present invention include alkylene chains:

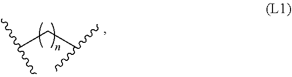

wherein n is an integer from 1-10, inclusive, e.g., 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10, examples of which include:

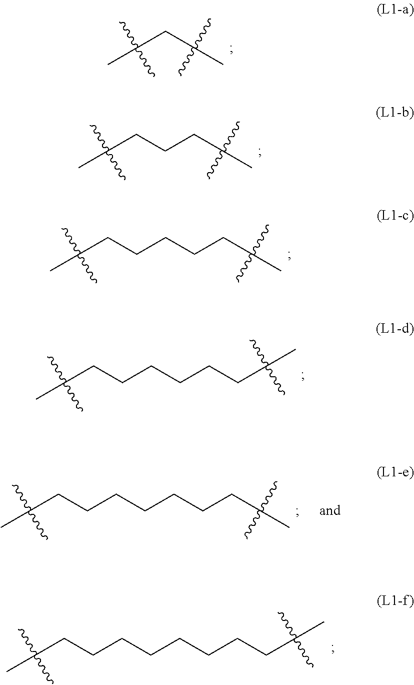

alkylene chains terminating in various functional groups (as described above), examples of which are as follows:

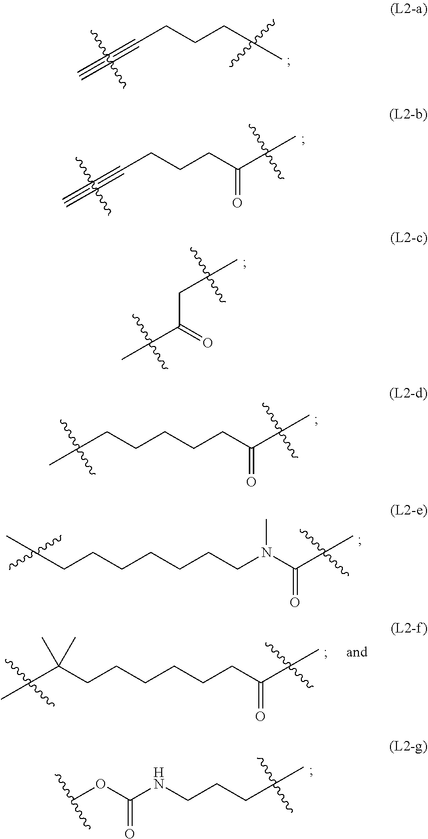

alkylene chains interrupted with various functional groups (as described above), examples of which are as follows:

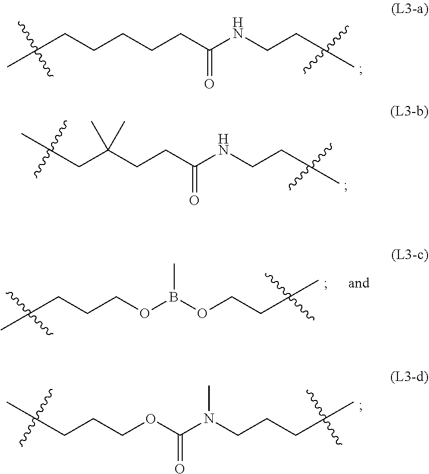 [SMILES: *CCCCCC(=O)NCC* ; *CCNC(=O)CCC(C)(C)C* ; *CCCOB(C)OCC* ; *CCCOC(=O)N(C)CCC*]

alkylene chains interrupted or terminating with heterocyclene groups, e.g.

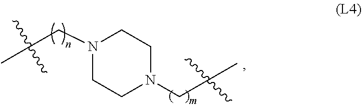

wherein m and n are independently integers of 0-10 examples of which include:

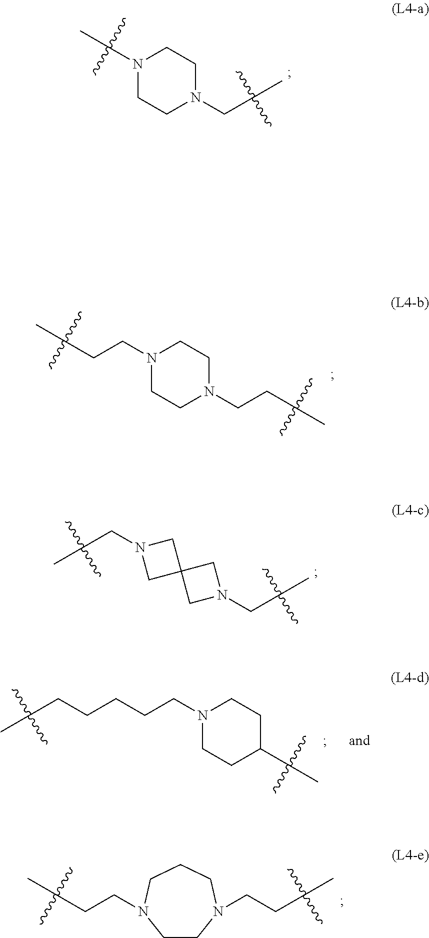 [SMILES: *CN1CCN(*)CC1 ; *CCN1CCN(CC*)CC1 ; *CN1CC2(C1)CN(C*)C2 ; *CCCCCN1CCC(*)CC1 ; *CCN1CCCN(CC*)CC1]

alkylene chains interrupted by amide, heterocyclene and/or aryl groups, examples of which include:

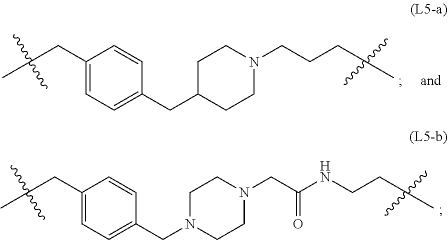 [SMILES: *CCCN1CCC(Cc2ccc(C*)cc2)CC1 ; *CCNC(=O)CN1CCN(Cc2ccc(C*)cc2)CC1]

alkylene chains interrupted by heterocyclene and aryl groups, and a heteroatom, examples of which include:

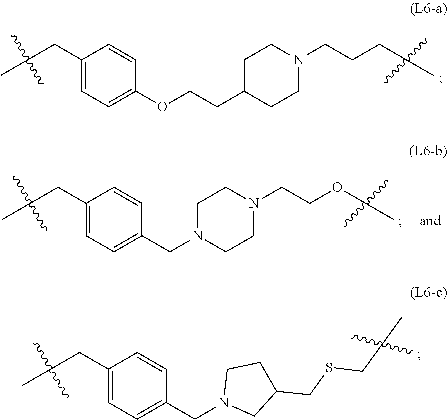 [SMILES: *CCCN1CCC(CCOc2ccc(C*)cc2)CC1 ; *Cc1ccc(CN2CCN(CCO*)CC2)cc1 ; *CSCC1CCN(Cc2ccc(C*)cc2)C1]

and
alkylene chains interrupted by a heteroatom such as N, O or B, e.g.,

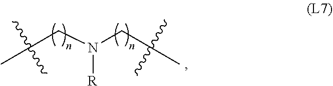

wherein n is an integer from 1-10, e.g., 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, and 1, 2, 3, 4, 5, 6, 7, 8, 9 and 10, and R is H, or C1 to C4 alkyl, an example of which is

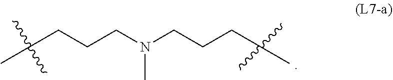 [SMILES: *CCCN(C)CCC*]

In some embodiments, the linker is a polyethylene glycol chain, examples of which include:

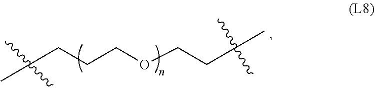

wherein n is an integer from 2-10, examples of which include:

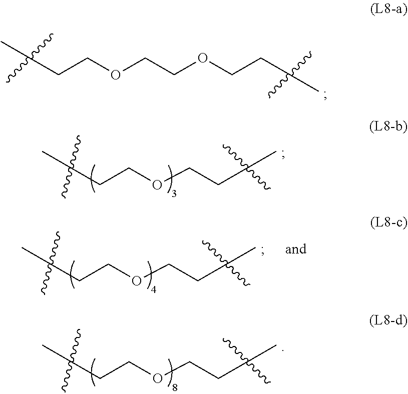

In some embodiments, the polyethylene glycol chain may terminate in a functional group, examples of which are as follows:

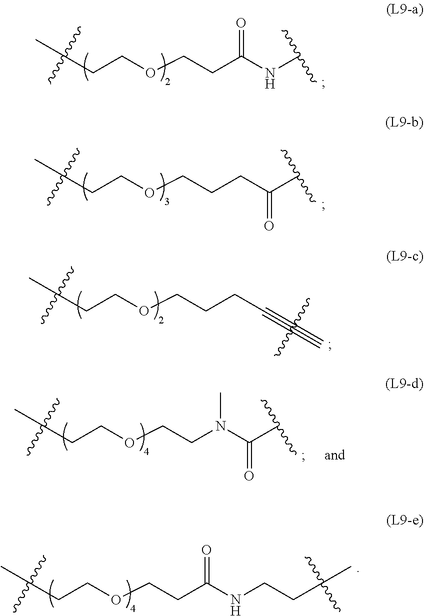

In some embodiments, the linker is represented by a structure selected from the group consisting of:

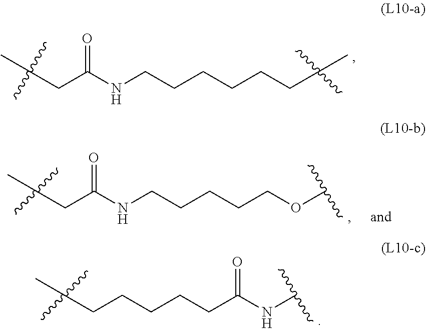 [SMILES: *CCCCCCNC(=O)C* ; *CC(=O)NCCCCCO* ; *CCCCCC(=O)N*]

In some embodiments, bifunctional compounds of the present invention may include a TL linked to a degron via an alkylene linker that is interrupted by and/or terminating in a non-cyclic functional group or one or more heteroatoms. Representative examples of bifunctional compounds include:

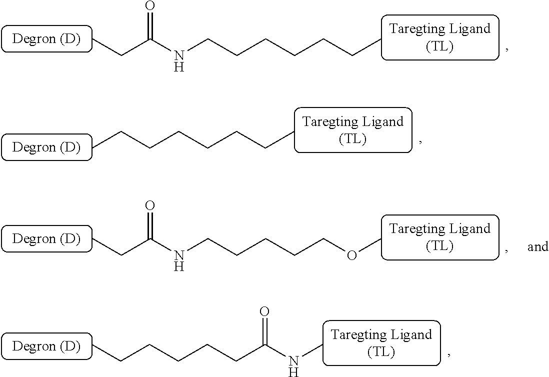

or a pharmaceutically acceptable salt or stereoisomer thereof.

In some embodiments, the bifunctional compounds of the present invention are represented by any of the following structures (with the Degron shown generically):

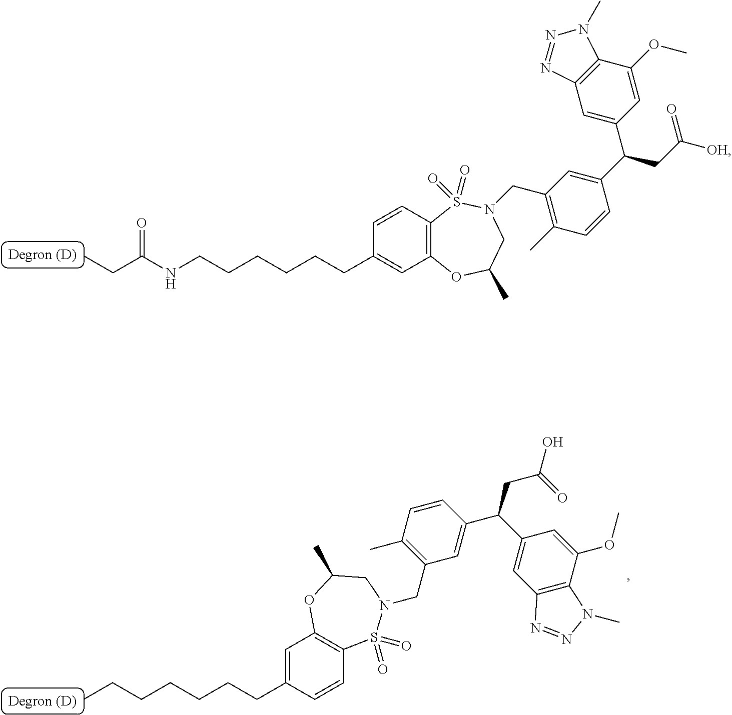

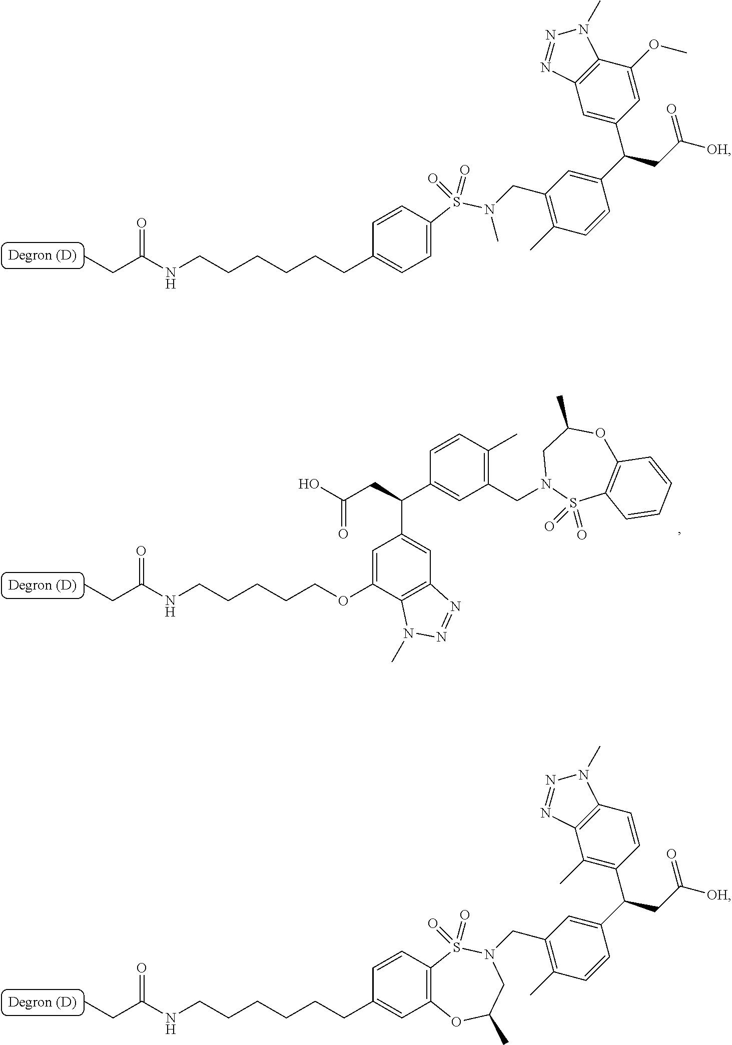

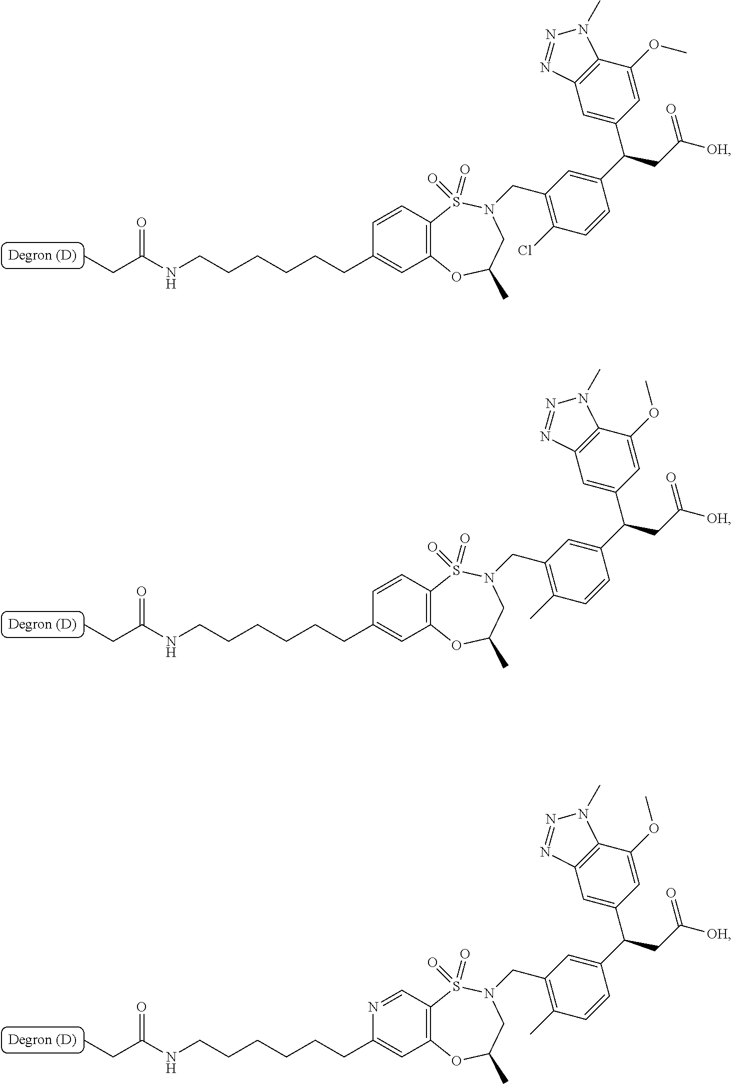

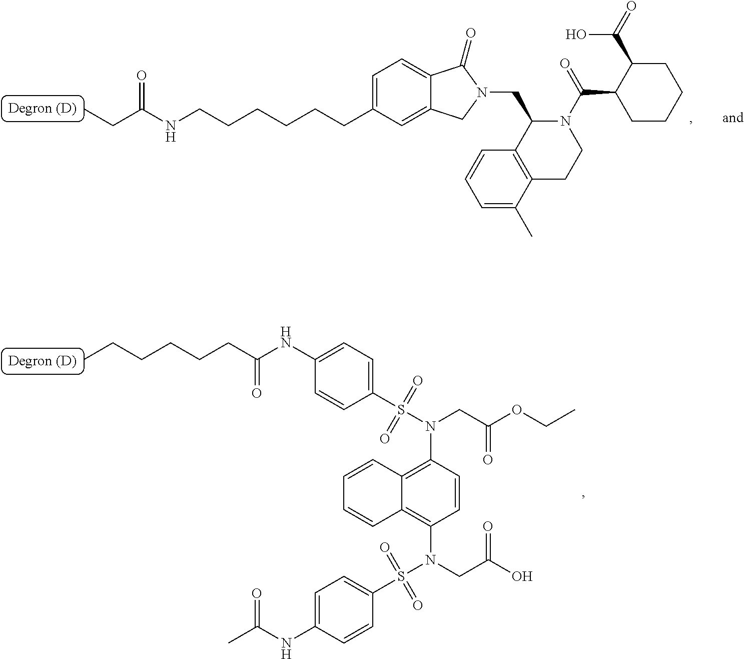

or a pharmaceutically acceptable salt or stereoisomer thereof.

### Degrons

The Ubiquitin-Proteasome Pathway (UPP) is a critical cellular pathway that regulates key regulator proteins and degrades misfolded or abnormal proteins. UPP is central to multiple cellular processes. The covalent attachment of ubiquitin to specific protein substrates is achieved through the action of E3 ubiquitin ligases. These ligases include over 500 different proteins and are categorized into multiple classes defined by the structural element of their E3 functional activity.

In some embodiments, the degron binds the E3 ubiquitin ligase which is cereblon and is represented by a structure selected from the group consisting of:

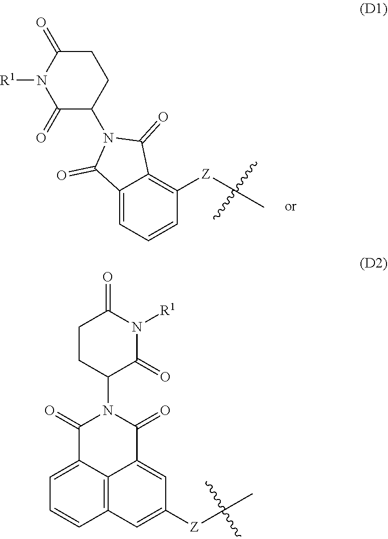

wherein

### R¹ is H or Me;

### Z is NH, O, or C≡.

Thus, in some embodiments, the compounds of this invention are represented by a formula selected from the group consisting of:

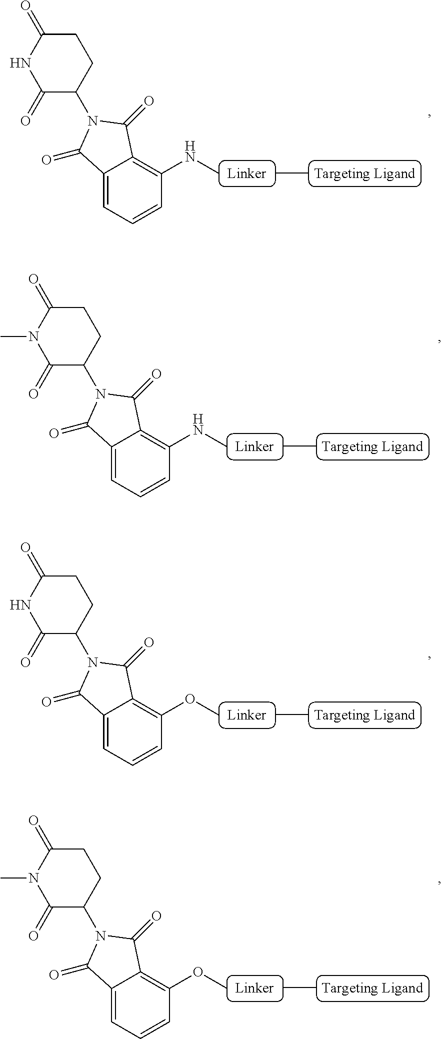

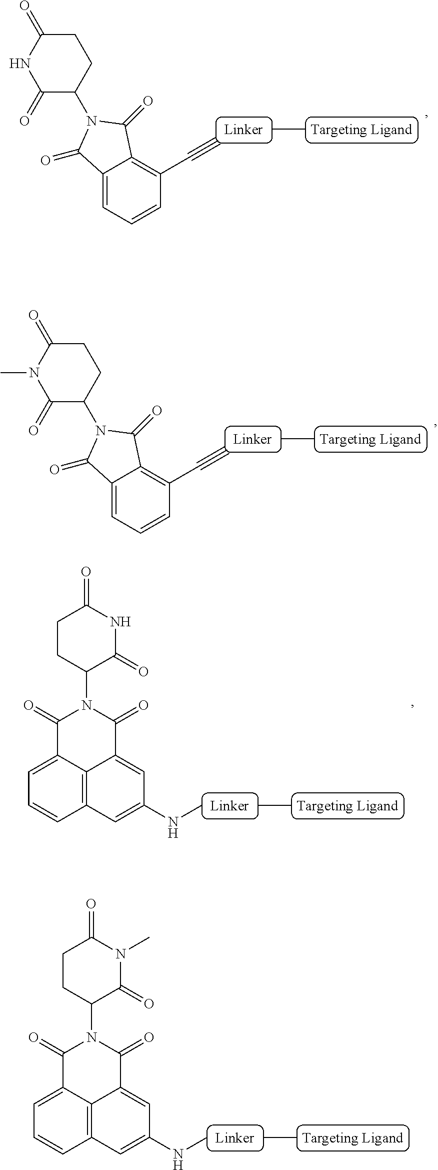

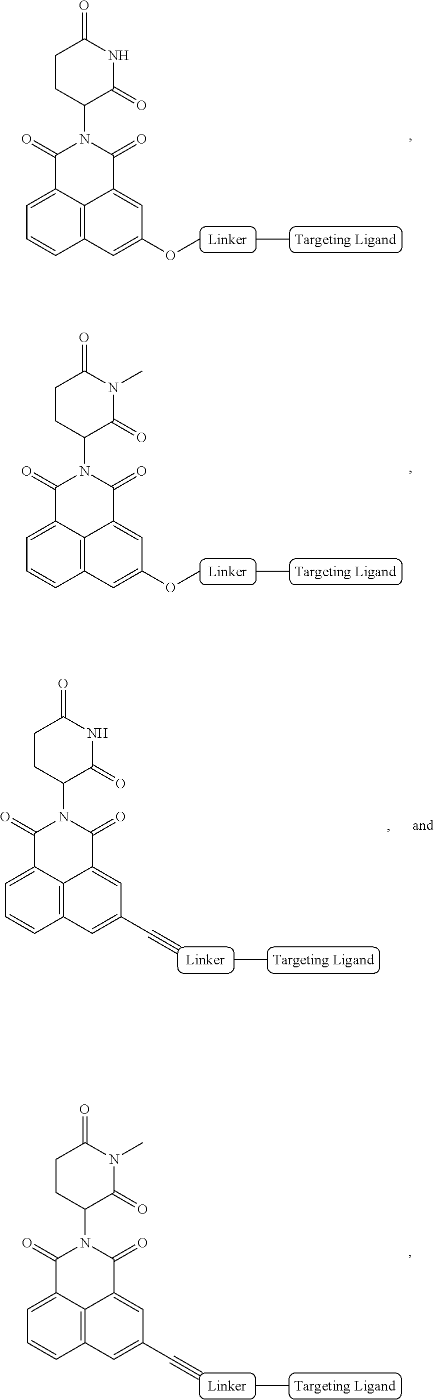

or a pharmaceutically acceptable salt or stereoisomer thereof.

Thus, in some embodiments, the compounds of this invention are represented by a formula selected from the group consisting of

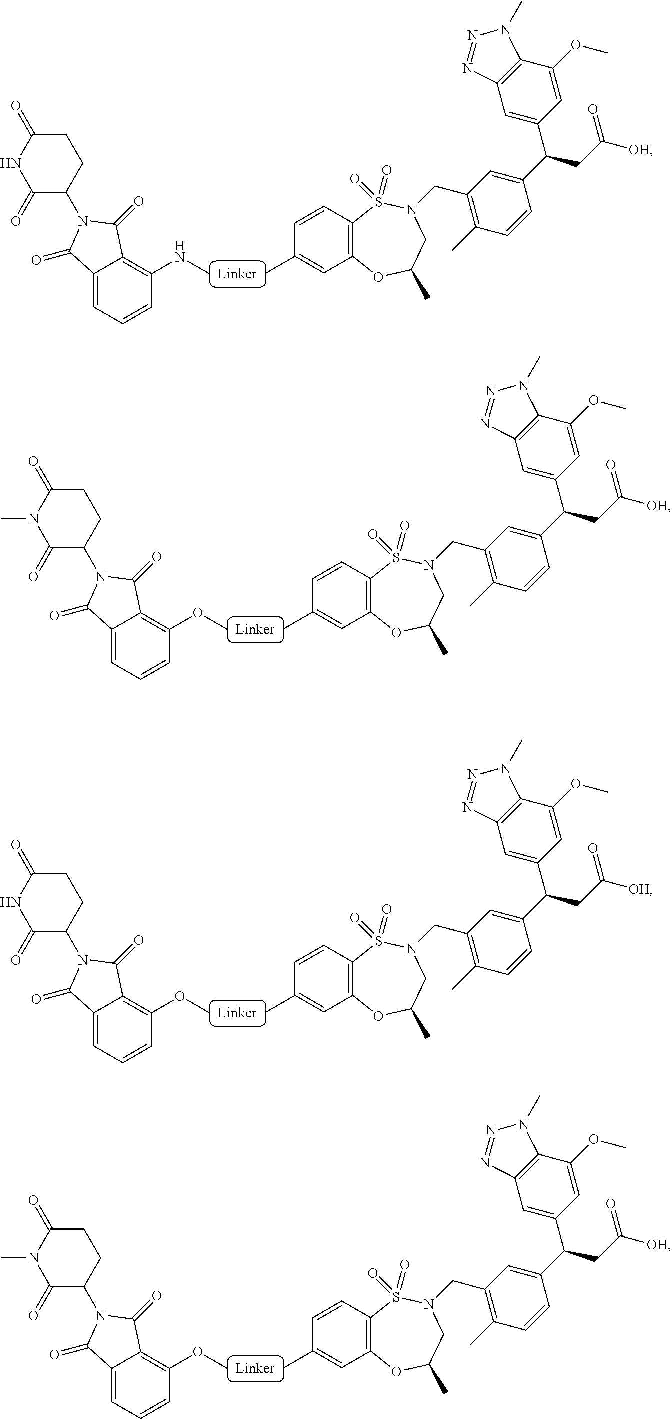

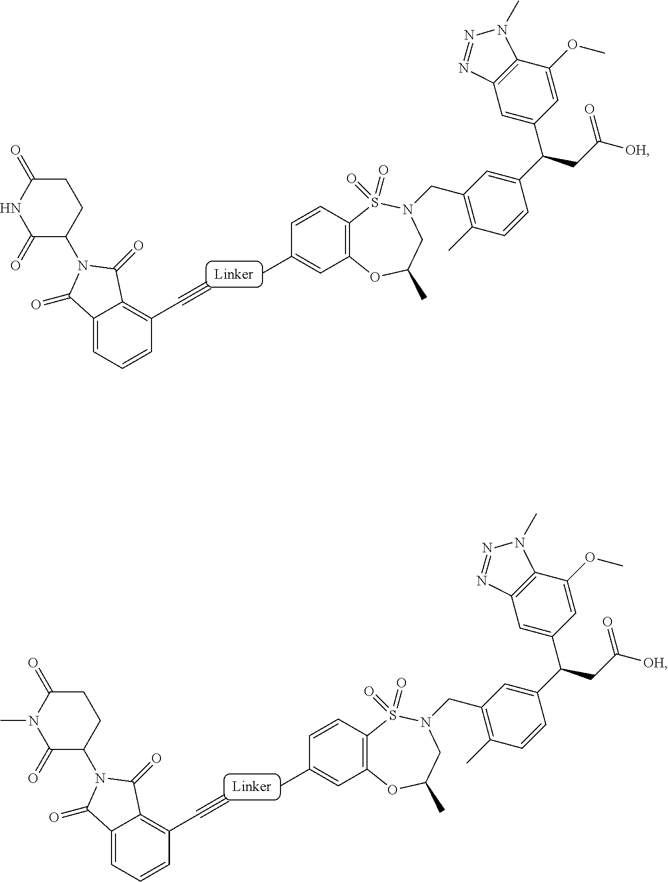 [SMILES: C#Cc1cccc2c1C(=O)N(C1CCC(=O)NC1=O)C2=O ; COc1cc([C@@H](CC(=O)O)c2ccc(C)c(CN3C[C@@H](C)Oc4cc(C)ccc4S3(=O)=O)c2)cc2nnn(C)c12 ; C#Cc1cccc2c1C(=O)N(C1CCC(=O)N(C)C1=O)C2=O ; COc1cc([C@@H](CC(=O)O)c2ccc(C)c(CN3C[C@@H](C)Oc4cc(C)ccc4S3(=O)=O)c2)cc2nnn(C)c12]

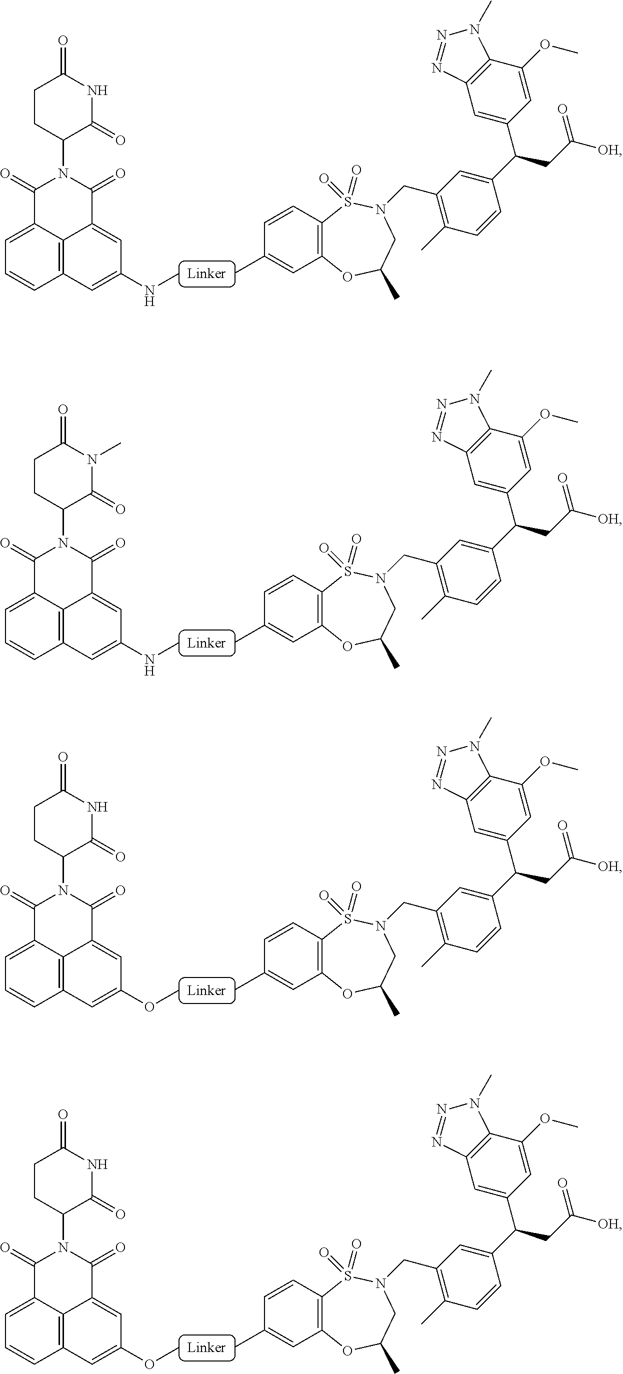

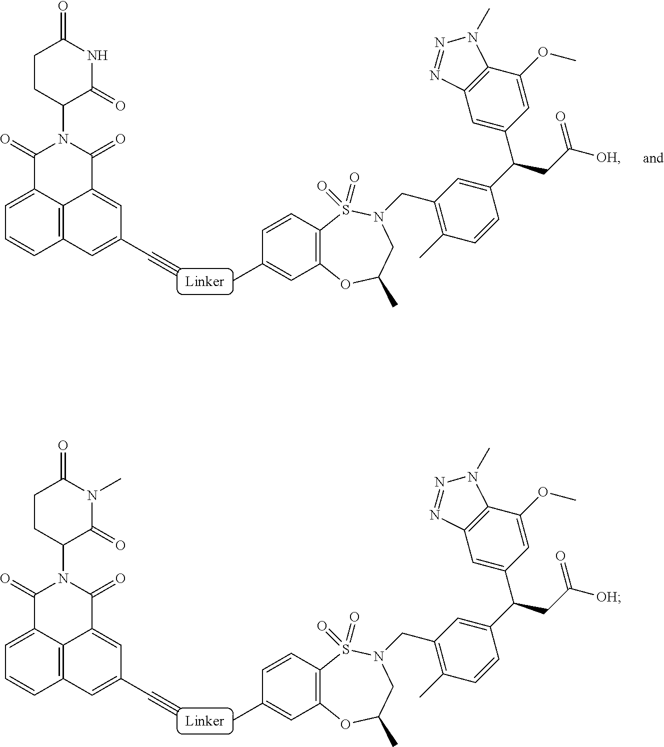 [SMILES: C#Cc1cc2c3c(cccc3c1)C(=O)N(C1CCC(=O)NC1=O)C2=O ; COc1cc([C@@H](CC(=O)O)c2ccc(C)c(CN3C[C@@H](C)Oc4cc(C)ccc4S3(=O)=O)c2)cc2nnn(C)c12 ; C#Cc1cc2c3c(cccc3c1)C(=O)N(C1CCC(=O)N(C)C1=O)C2=O ; COc1cc([C@@H](CC(=O)O)c2ccc(C)c(CN3C[C@@H](C)Oc4cc(C)ccc4S3(=O)=O)c2)cc2nnn(C)c12]

or a pharmaceutically acceptable salt or stereoisomer thereof.

Yet other degrons that bind cereblon and which may be suitable for use in the present invention are disclosed in U.S. Pat. No. 9,770,512, and U.S. Patent Application Publication Nos. 2018/0015087, 2018/0009779, 2016/0243247, 2016/0235731, 2016/0235730, and 2016/0176916, and International Patent Publications WO 2017/197055, WO 2017/197051, WO 2017/197036, WO 2017/197056 and WO 2017/197046.

In some embodiments, the E3 ubiquitin ligase that is bound by the degron is the von Hippel-Lindau (VHL) tumor suppressor. See, Iwai, et al., Proc. Nat'l. Acad. Sci. USA 96:12436-41 (1999).

In some embodiments, the degrons that bind VHL are represented by the following formulas:

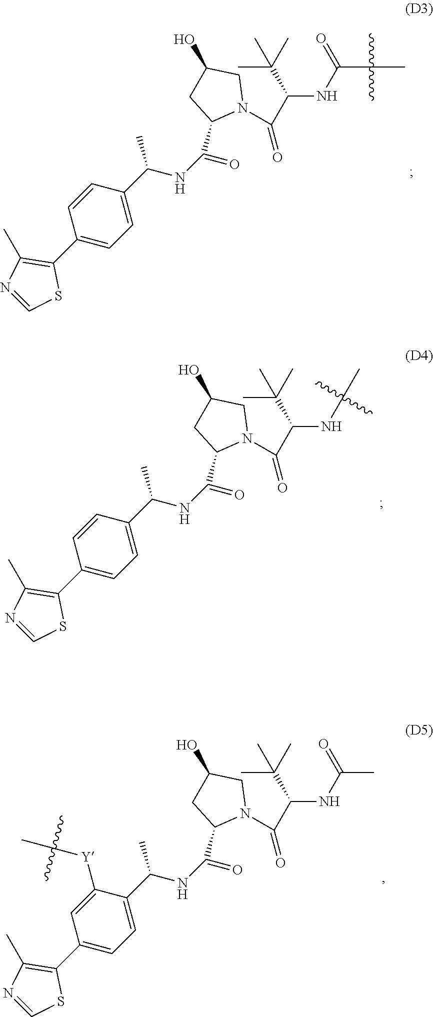

wherein Y′ is a bond, N, O or C;

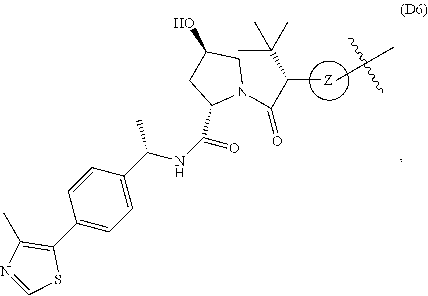

wherein Z is a cyclic group, which in
some embodiments is a C5-6 carbocyclic or heterocyclic group. In certain embodiments, the heterocyclic group is

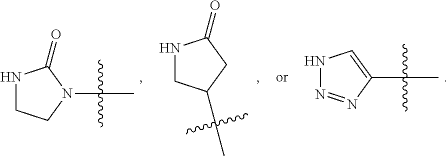 [SMILES: *N1CCNC1=O ; *C1CNC(=O)C1 ; *c1c[nH]nn1]

In some embodiments, the present invention provides a compound represented by any of the following formulas:

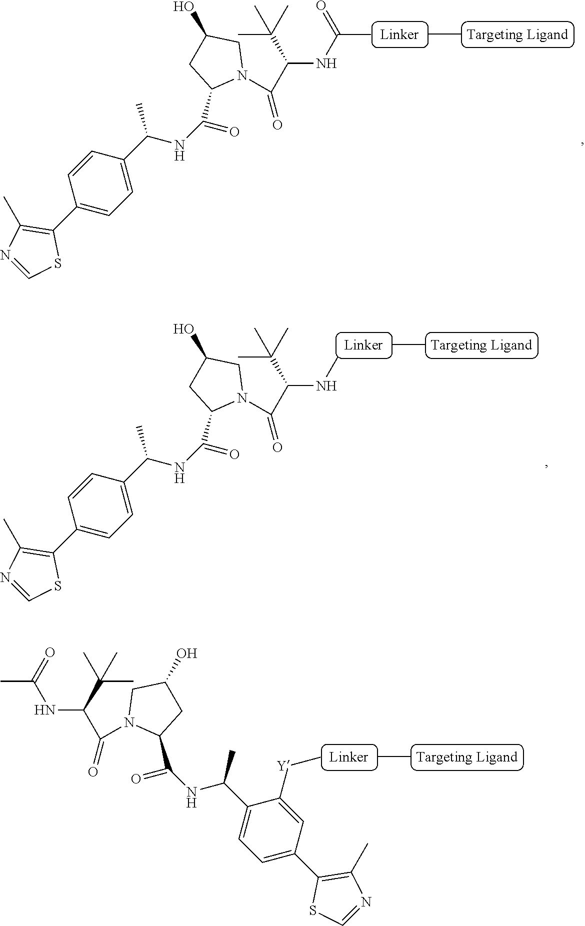

wherein Y′ is a bond, NH, O or CH₂,

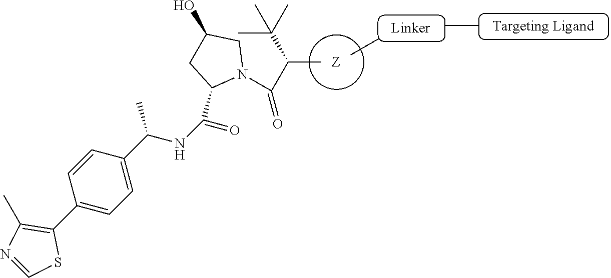

wherein Z is a cyclic group, or a pharmaceutically acceptable salt or stereoisomer thereof.

In some embodiments, the cyclic group is preferably phenyl, pyrrolyl, furanyl, thiophenyl, pyrazolyl, imidazolyl, oxazolyl, thiazolyl, pyridinyl, pyridazinyl, pyrimidinyl, benzofuranyl, benzothiophenyl, indolyl, quinolinyl, or isoquinolinyl.

Yet other degrons that bind VHL and which may be suitable for use in the present invention are disclosed in U.S. Patent Application Publication 2017/0121321 A1.

Thus, in some embodiments, the compounds of this invention are represented by any structures generated by the combination of structures TL1 to TL4, L1 to L10, and the structures of the degrons described herein, including D1 to D6, or a pharmaceutically acceptable salt or stereoisomer thereof.

The above structures are representative compounds of the present invention that contain cereblon targeted degrons (D1 and D2). VHL targeted degrons (D3 to D6) can be substituted for the cereblon targeted degrons (D1 and D2) in the above structures to represent further compounds of the present invention.

Further representative compounds of the present invention are represented by the following structures:

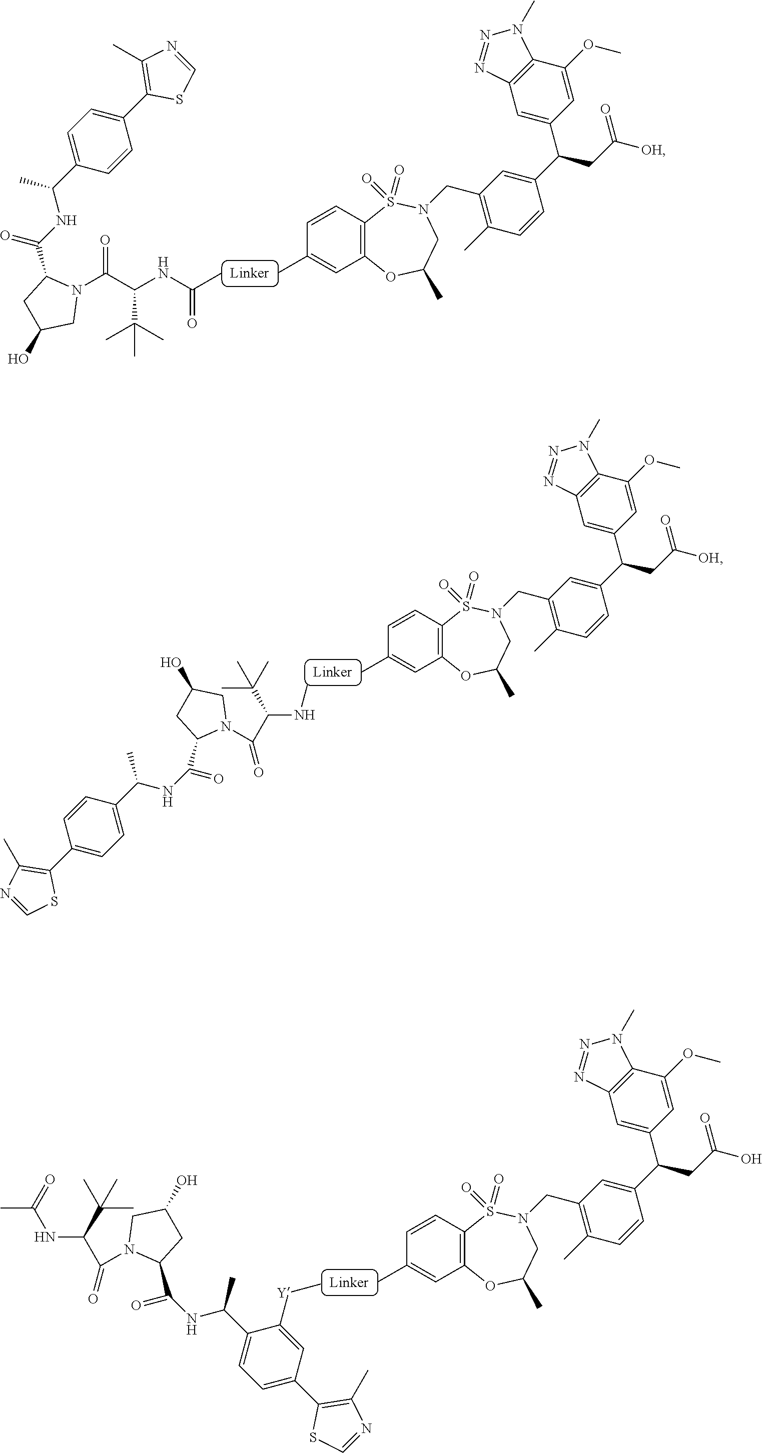

wherein Y′ is a bond, N, O or C, and

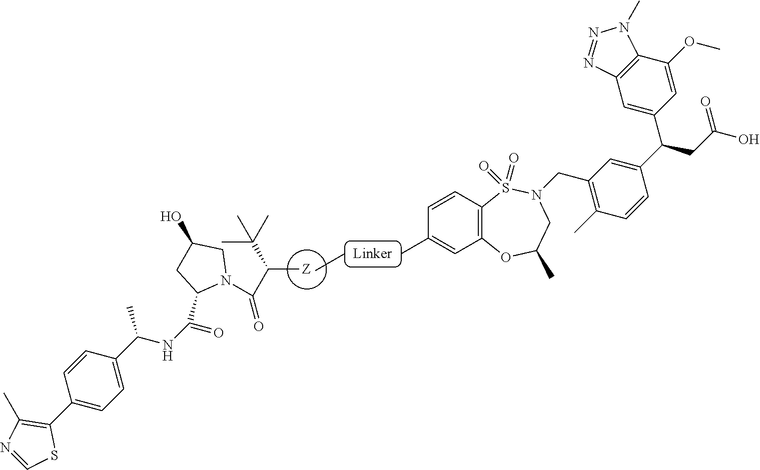

wherein Z is a cyclic group, or a pharmaceutically acceptable salt or stereoisomer thereof.

In some embodiments, the compounds of the present invention have the following structures:

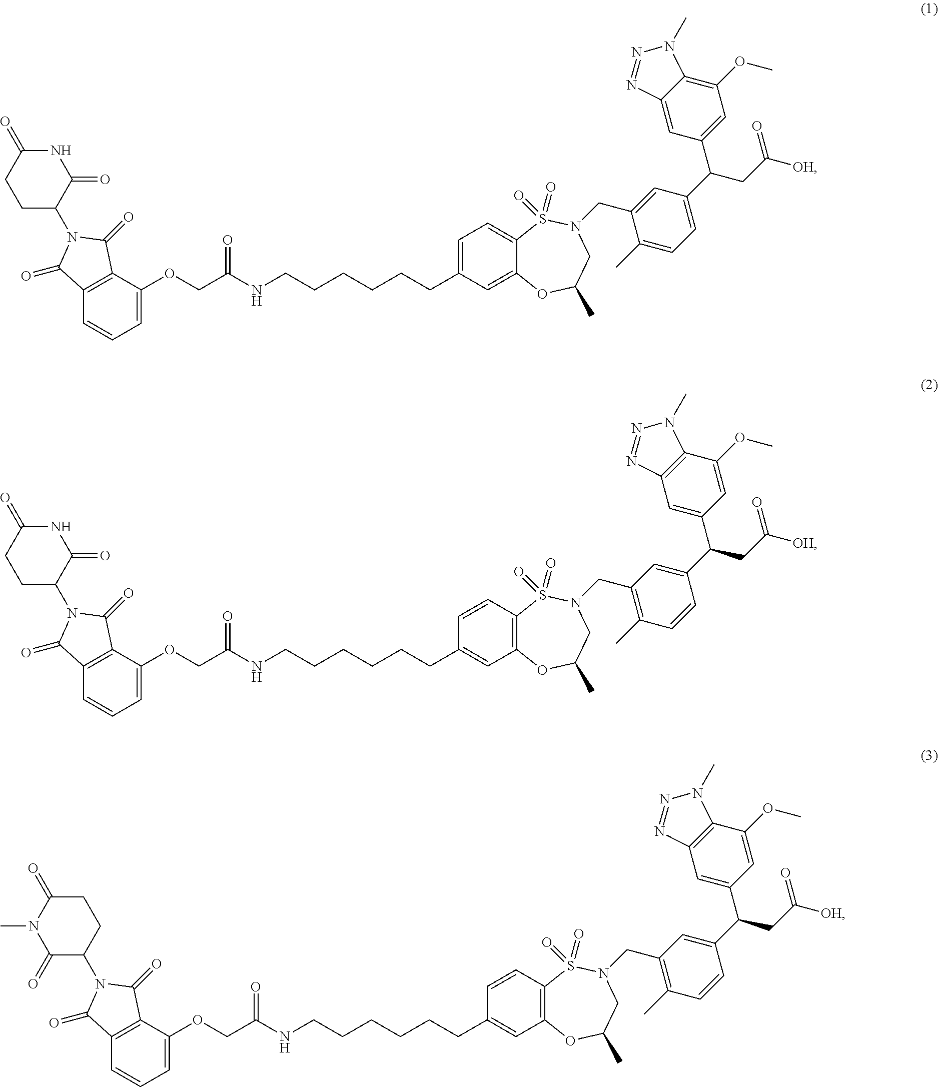 [SMILES: COc1cc(C(CC(=O)O)c2ccc(C)c(CN3C[C@@H](C)Oc4cc(CCCCCCNC(=O)COc5cccc6c5C(=O)N(C5CCC(=O)NC5=O)C6=O)ccc4S3(=O)=O)c2)cc2nnn(C)c12 ; COc1cc([C@@H](CC(=O)O)c2ccc(C)c(CN3C[C@@H](C)Oc4cc(CCCCCCNC(=O)COc5cccc6c5C(=O)N(C5CCC(=O)NC5=O)C6=O)ccc4S3(=O)=O)c2)cc2nnn(C)c12 ; COc1cc([C@@H](CC(=O)O)c2ccc(C)c(CN3C[C@@H](C)Oc4cc(CCCCCCNC(=O)COc5cccc6c5C(=O)N(C5CCC(=O)N(C)C5=O)C6=O)ccc4S3(=O)=O)c2)cc2nnn(C)c12]

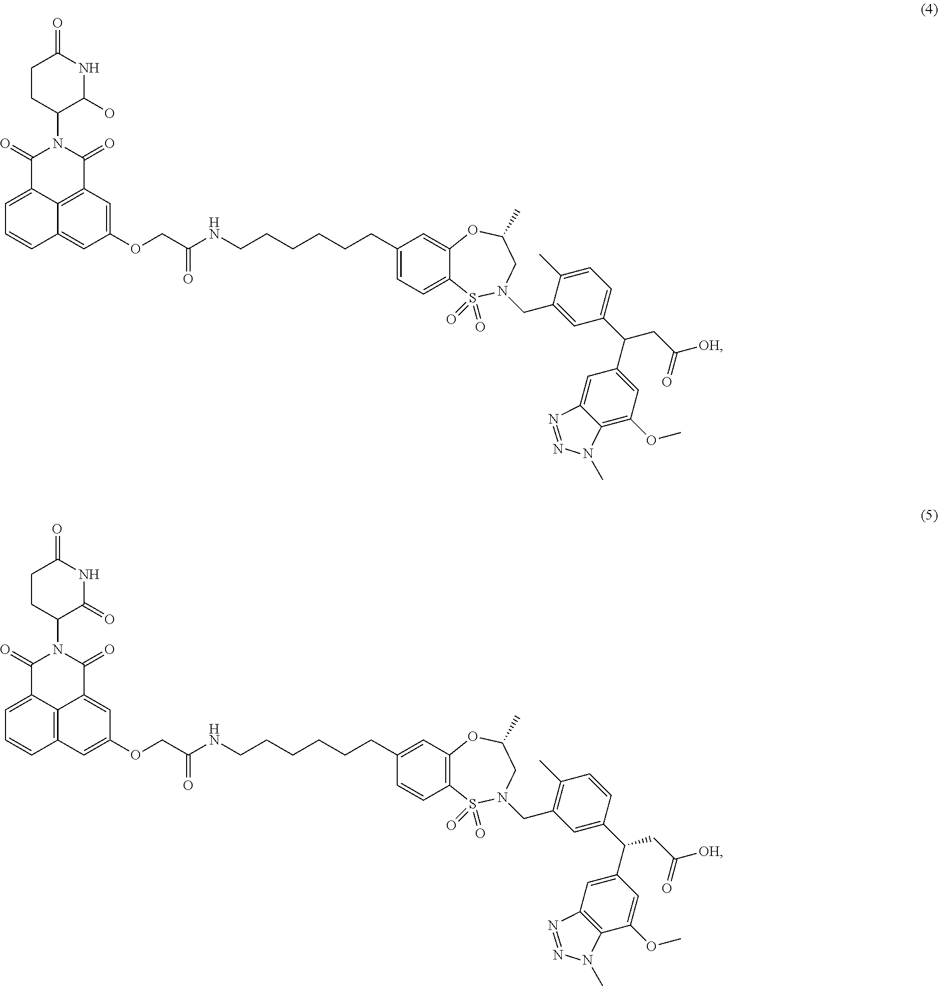 [SMILES: COc1cc(C(CC(=O)O)c2ccc(C)c(CN3C[C@@H](C)Oc4cc(CCCCCCNC(=O)COc5cc6c7c(cccc7c5)C(=O)N(C5CCC(=O)NC5O)C6=O)ccc4S3(=O)=O)c2)cc2nnn(C)c12 ; COc1cc([C@@H](CC(=O)O)c2ccc(C)c(CN3C[C@@H](C)Oc4cc(CCCCCCNC(=O)COc5cc6c7c(cccc7c5)C(=O)N(C5CCC(=O)NC5=O)C6=O)ccc4S3(=O)=O)c2)cc2nnn(C)c12]

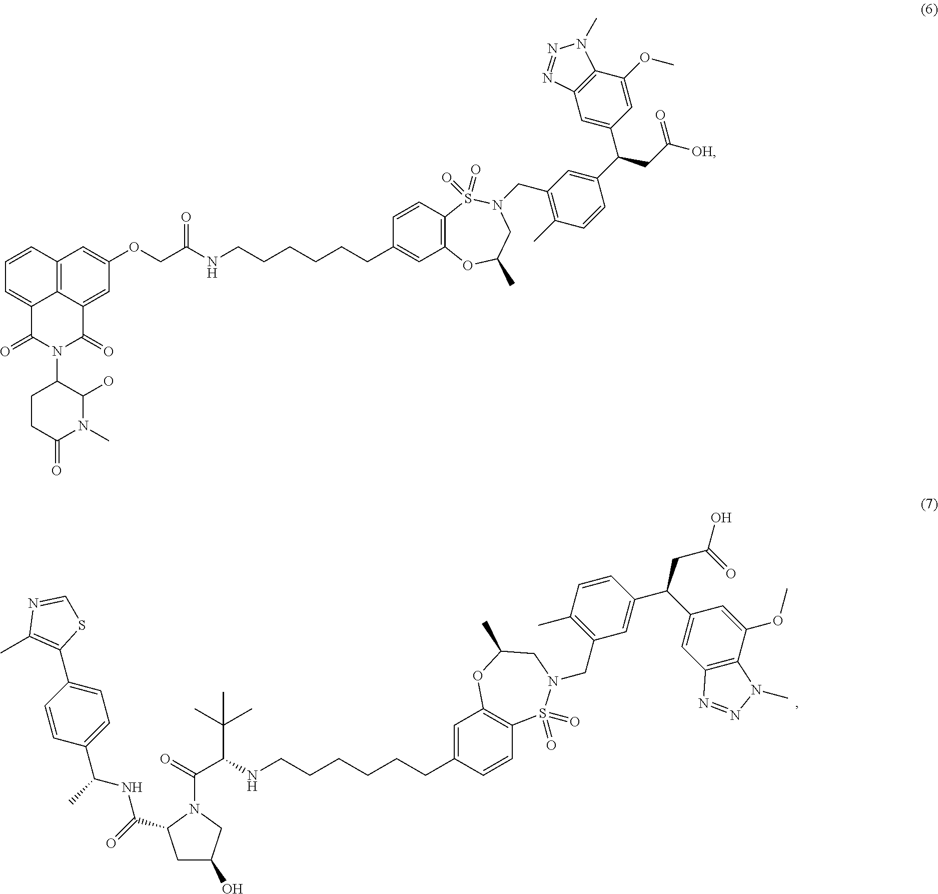

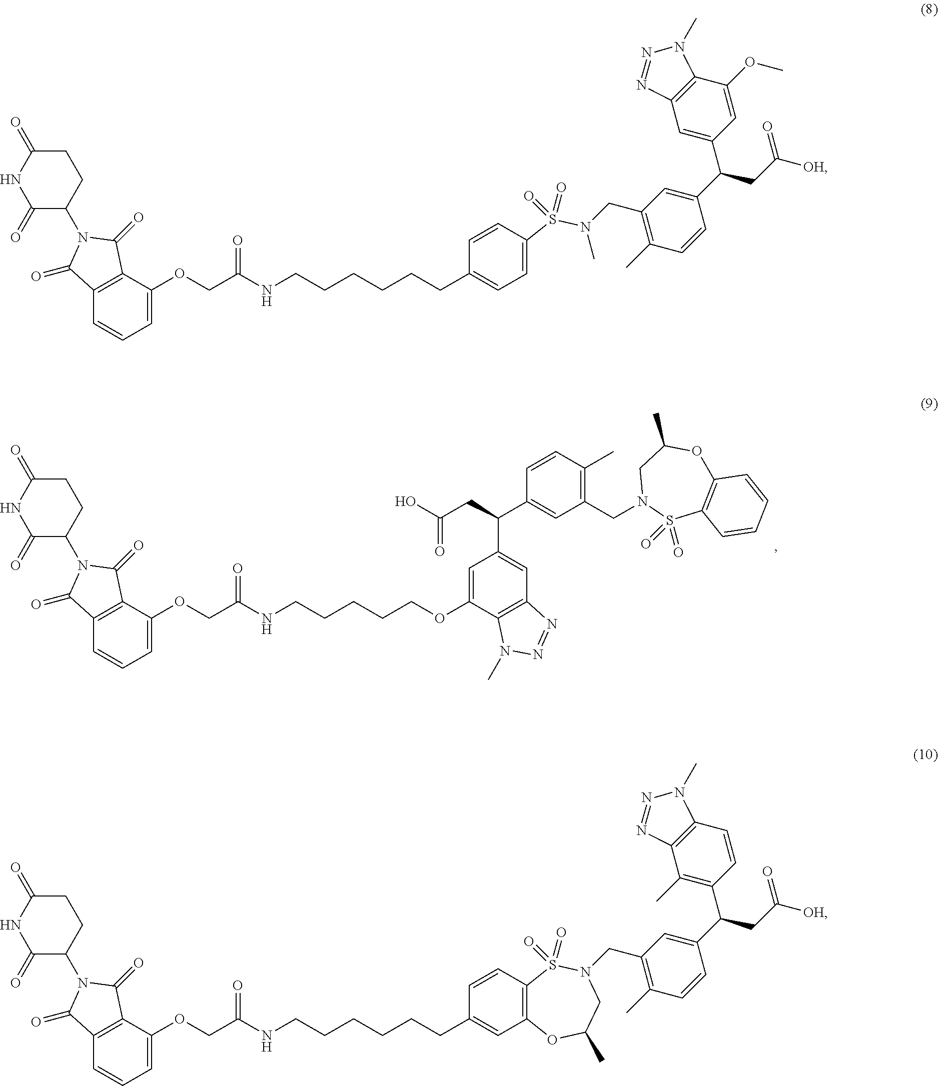 [SMILES: COc1cc([C@@H](CC(=O)O)c2ccc(C)c(CN(C)S(=O)(=O)c3ccc(CCCCCCNC(=O)COc4cccc5c4C(=O)N(C4CCC(=O)NC4=O)C5=O)cc3)c2)cc2nnn(C)c12 ; Cc1ccc([C@H](CC(=O)O)c2cc(OCCCCCNC(=O)COc3cccc4c3C(=O)N(C3CCC(=O)NC3=O)C4=O)c3c(c2)nnn3C)cc1CN1C[C@@H](C)Oc2ccccc2S1(=O)=O ; Cc1ccc([C@H](CC(=O)O)c2ccc3c(nnn3C)c2C)cc1CN1C[C@@H](C)Oc2cc(CCCCCCNC(=O)COc3cccc4c3C(=O)N(C3CCC(=O)NC3=O)C4=O)ccc2S1(=O)=O]

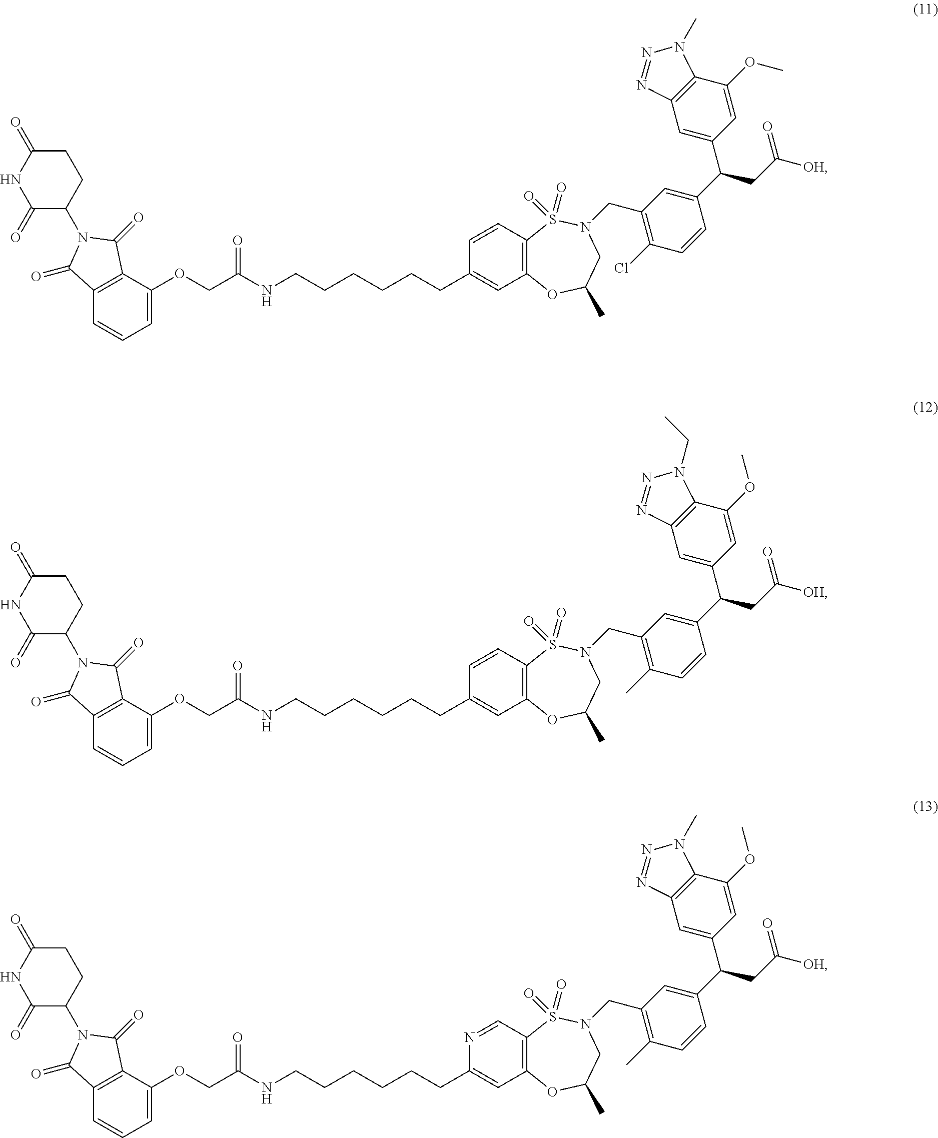 [SMILES: COc1cc([C@@H](CC(=O)O)c2ccc(Cl)c(CN3C[C@@H](C)Oc4cc(CCCCCCNC(=O)COc5cccc6c5C(=O)N(C5CCC(=O)NC5=O)C6=O)ccc4S3(=O)=O)c2)cc2nnn(C)c12 ; CCn1nnc2cc([C@@H](CC(=O)O)c3ccc(C)c(CN4C[C@@H](C)Oc5cc(CCCCCCNC(=O)COc6cccc7c6C(=O)N(C6CCC(=O)NC6=O)C7=O)ccc5S4(=O)=O)c3)cc(OC)c21 ; COc1cc([C@@H](CC(=O)O)c2ccc(C)c(CN3C[C@@H](C)Oc4cc(CCCCCCNC(=O)COc5cccc6c5C(=O)N(C5CCC(=O)NC5=O)C6=O)ncc4S3(=O)=O)c2)cc2nnn(C)c12]

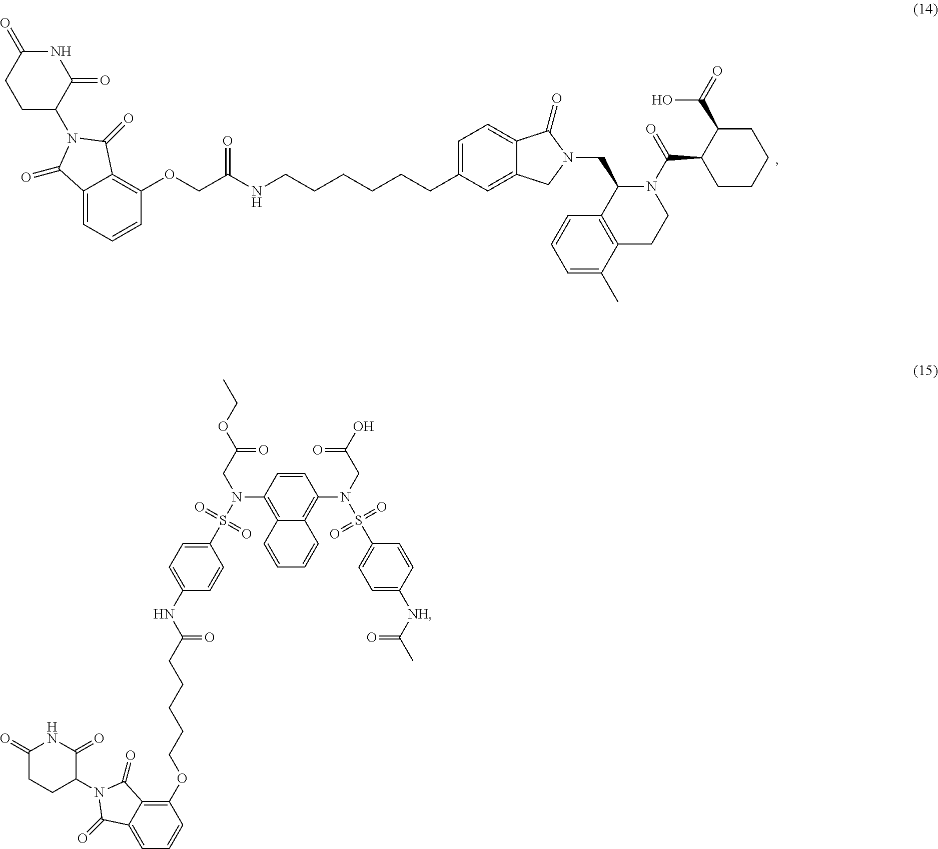 [SMILES: Cc1cccc2c1CCN(C(=O)[C@@H]1CCCC[C@@H]1C(=O)O)[C@@H]2CN1Cc2cc(CCCCCCNC(=O)COc3cccc4c3C(=O)N(C3CCC(=O)NC3=O)C4=O)ccc2C1=O ; CCOC(=O)CN(c1ccc(N(CC(=O)O)S(=O)(=O)c2ccc(NC(C)=O)cc2)c2ccccc12)S(=O)(=O)c1ccc(NC(=O)CCCCCOc2cccc3c2C(=O)N(C2CCC(=O)NC2=O)C3=O)cc1]

or a pharmaceutically acceptable salt or stereoisomer thereof.

Bifunctional compounds of the present invention may be in the form of a free acid or free base, or a pharmaceutically acceptable salt. As used herein, the term “pharmaceutically acceptable” refers to a material, such as a carrier or diluent, which does not abrogate the biological activity or properties of the compound, and is relatively non-toxic, i.e., the material may be administered to a subject without causing undesirable biological effects (such as dizziness or gastric upset) or interacting in a deleterious manner with any of the components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain bifunctional compounds of the invention can form pharmaceutically acceptable salts with various organic bases such as lysine, arginine, guanidine, diethanolamine or metformin.

In some embodiments, the bifunctional compound of the present invention is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

Bifunctional compounds of the present invention may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present invention may be made and used in the form of individual isomers and substantially free of other isomers, or in the form of a mixture of various isomers, e.g., racemic mixtures of stereoisomers.

### Methods of Synthesis

In another aspect, the present invention is directed to a method for making a bifunctional compound of formula I, or a pharmaceutically acceptable salt or stereoisomer thereof. Broadly, the inventive compounds or pharmaceutically-acceptable salts or stereoisomers thereof may be prepared by any process known to be applicable to the preparation of chemically related compounds. The compounds of the present invention will be better understood in connection with the synthetic schemes that described in various working examples and which illustrate non-limiting methods by which the bifunctional compounds may be prepared.

### Pharmaceutical Compositions

In some embodiments, the present invention is directed to a pharmaceutical composition that includes a therapeutically effective amount of the bifunctional compound of formula I or a pharmaceutically acceptable salt or stereoisomer thereof, and a pharmaceutically acceptable carrier. The bifunctional compounds of the present invention may be formulated into several different types of pharmaceutical compositions that contain a therapeutically effective amount of the compound, and a pharmaceutically acceptable carrier.

Broadly, bifunctional compounds of formula I and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral), parenteral (e.g., subcutaneous (s.c.), intravenous (i.v.), intramuscular (i.m.), and intrasternal injection, or infusion techniques, intra-arterial, intramedullary, intrathecal, intraventricular, transdermal, interdermal, rectal, intravaginal, intraperitoneal, topical mucosal, nasal, buccal, sublingual, intratracheal instillation, bronchial instillation, and/or inhalation. In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). In some embodiments, the compositions are formulated for oral or intravenous administration (e.g., systemic intravenous injection).

The term “pharmaceutically acceptable carrier,” as known in the art, refers to a pharmaceutically acceptable material, composition or vehicle, suitable for administering bifunctional compounds of the present invention to mammals. Suitable carriers may include, for example, liquids (both aqueous and non-aqueous alike, and combinations thereof), solids, encapsulating materials, gases, and combinations thereof (e.g., semi-solids), that function to carry or transport the compound from one organ, or portion of the body, to another organ or portion of the body. A carrier is “acceptable” in the sense of being physiologically inert to and compatible with the other ingredients of the formulation, and which is non-toxic to the subject or patient. Depending on the type of formulation, the composition may further include one or more pharmaceutically acceptable excipients.

Accordingly, bifunctional compounds of formula I and their pharmaceutically acceptable salts or stereoisomers may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, bifunctional compounds of formula I may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

In some embodiments, bifunctional compounds of formula I may be formulated into tablets that may include excipients such as lactose monohydrate, microcrystalline cellulose, sodium starch glycolate, magnesium tartrate, and hydrophobic colloidal silica.

They may be formulated as solutions for parenteral and oral delivery forms, particularly to the extent that they are water-soluble. Parenteral administration may also be advantageous in that the compound may be administered relatively quickly such as in the case of a single-dose treatment and/or an acute condition.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, bifunctional compounds of formula I may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

The compositions may be formulated for buccal or sublingual administration, examples of which include tablets, lozenges and gels.

The bifunctional compounds of formula I may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Bifunctional compounds of formula I may be formulated for topical administration which as used herein, refers to administration intradermally by invention of the formulation to the epidermis. These types of compositions are typically in the form of ointments, pastes, creams, lotions, gels, solutions and sprays.

Representative examples of carriers useful in formulating compositions for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

As used herein, the term, “therapeutically effective amount” refers to an amount of a bifunctional compound of formula I or a pharmaceutically acceptable salt or a stereoisomer thereof that is effective in producing the desired therapeutic response in a particular patient suffering from a disease or disorder. The term “therapeutically effective amount” thus includes the amount of the compound of the invention or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased (e.g., cancer) cells, or reduces the amount of KEAP1 in diseased cells.

The total daily dosage of the inventive bifunctional compounds and usage thereof may be decided in accordance with standard medical practice, e.g., by the attending physician using sound medical judgment. Accordingly, the specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the specific compound employed; and like factors well known in the medical arts (see, for example, Goodman and Gilman's, “The Pharmacological Basis of Therapeutics”, 10th Edition, A. Gilman, J. Hardman and L. Limbird, eds., McGraw-Hill Press, 155-173, 2001).

Bifunctional compounds of formula I and their pharmaceutically acceptable salts and stereoisomers may be effective over a wide dosage range. In some embodiments, the total daily dosage (e.g., for adult humans) may range from about 0.001 to about 1000 mg, from 0.01 to about 1000 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, or in yet other embodiments from about 10 to about 30 mg per day. In some embodiments, the total daily dosage may range from 400 mg to 600 mg. Individual dosages may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day.

### Methods of Use

In some aspects, the present invention is directed to methods of treating diseases or disorders involving dysfunctional (e.g., dysregulated) KEAP1 activity, that entails administration of a therapeutically effective amount of a bifunctional compound of formula I or a pharmaceutically acceptable salt or stereoisomer thereof, to a subject in need thereof.

The diseases or disorders may be said to be characterized or mediated by dysfunctional KEAP1 activity (e.g., elevated levels of protein or otherwise functionally abnormal relative to a non-pathological state). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health. In some embodiments, compounds of the invention may be useful in the treatment of cell proliferative diseases and disorders (e.g., cancer or benign neoplasms). As used herein, the term “cell proliferative disease or disorder” refers to the conditions characterized by deregulated or abnormal cell growth, or both, including noncancerous conditions such as neoplasms, precancerous conditions, benign tumors, and cancer.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

Exemplary types of non-cancerous (e.g., cell proliferative) diseases or disorders that may be amenable to treatment with the bifunctional compounds of the present invention include inflammatory diseases and conditions, autoimmune diseases, neurodegenerative diseases, heart diseases, viral diseases, chronic and acute kidney diseases or injuries, vascular diseases, metabolic diseases, and allergic and genetic diseases.

Representative examples of specific non-cancerous diseases and disorders include rheumatoid arthritis, alopecia areata, lymphoproliferative conditions, autoimmune hematological disorders (e.g. hemolytic anemia, aplastic anemia, anhidrotic ecodermal dysplasia, pure red cell anemia and idiopathic thrombocytopenia), cholecystitis, acromegaly, rheumatoid spondylitis, osteoarthritis, gout, scleroderma, sepsis, septic shock, dacryoadenitis, cryopyrin associated periodic syndrome (CAPS), endotoxic shock, endometritis, gram-negative sepsis, keratoconjunctivitis sicca, toxic shock syndrome, asthma, adult respiratory distress syndrome, chronic obstructive pulmonary disease, chronic pulmonary inflammation, chronic graft rejection, hidradenitis suppurativa, inflammatory bowel disease, Crohn's disease, Behcet's syndrome, systemic lupus erythematosus, multiple sclerosis, juvenile-onset diabetes, autoimmune uveoretinitis, autoimmune vasculitis, thyroiditis, Addison's disease, lichen planus, appendicitis, bullous pemphigus, pemphigus vulgaris, pemphigus foliaceus, paraneoplastic pemphigus, myasthenia gravis, immunoglobulin A nephropathy, autoimmune thyroiditis or Hashimoto's disease, Sjogren's syndrome, vitiligo, Wegener granulomatosis, granulomatous orchitis, autoimmune oophoritis, sarcoidosis, rheumatic carditis, ankylosing spondylitis, Grave's disease, autoimmune thrombocytopenic purpura, psoriasis, psoriatic arthritis, eczema, dermatitis herpetiformis, ulcerative colitis, pancreatic fibrosis, hepatitis, hepatic fibrosis, CD14 mediated sepsis, non-CD14 mediated sepsis, acute and chronic renal disease, irritable bowel syndrome, pyresis, restenosis, cerebral malaria, cervicitis, stroke and ischemic injury, neural trauma, acute and chronic pain, allergic rhinitis, allergic conjunctivitis, chronic heart failure, congestive heart failure, acute coronary syndrome, cachexia, malaria, leprosy, leishmaniasis, Lyme disease, Reiter's syndrome, acute synovitis, muscle degeneration, bursitis, tendonitis, tenosynovitis, herniated, ruptured, or prolapsed intervertebral disk syndrome, osteopetrosis, thrombosis, restenosis, silicosis, pulmonary sarcosis, bone resorption diseases, such as osteoporosis, graft-versus-host reaction, fibromyalgia, AIDS and other viral diseases such as Herpes Zoster, Herpes Simplex I or II, influenza virus and cytomegalovirus, diabetes Type I and II, obesity, insulin resistance and diabetic retinopathy, 22q11.2 deletion syndrome, Angelman syndrome, Canavan disease, celiac disease, Charcot-Marie-Tooth disease, color blindness, Cri du chat, Down syndrome, cystic fibrosis, Duchenne muscular dystrophy, haemophilia, Klinefleter's syndrome, neurofibromatosis, phenylketonuria, Prader-Willi syndrome, sudden infant death syndrome, sickle cell disease, Tay-Sachs disease, Turner syndrome, urea cycle disorders, thalassemia, otitis, pancreatitis, parotitis, pericarditis, peritonitis, pharyngitis, pleuritis, phlebitis, pneumonitis, cystic fibrosis, uveitis, polymyositis, proctitis, interstitial lung fibrosis, dermatomyositis, arteriosclerosis, atherosclerosis, amyotrophic lateral sclerosis, asocality, immune response, vaginitis, including chronic recurrent yeast vaginitis, depression, Sudden Infant Death Syndrome, obesity and varicosis.

In some embodiments, the cardiovascular disease is coronary artery disease, angina pectoris, peripheral artery disease, heart arrhythmia, cardiomyopathy, atrial fibrillation, rheumatic fever, heart valve disease, syncope, embolism, hypertensive heart disease, endocarditis, myocarditis, or aortic stenosis.

In some embodiments, the chronic obstructive pulmonary disease is emphysema, chronic bronchitis, refractory asthma, or bronchiectasis.

In some embodiments, the chronic kidney disease is uremia, glomerulonephritis, nephritis, acidosis, urinary tract infection, renal osteodystrophy, or interstitial nephritis.

In some embodiments, the neurological disease is Parkinson's disease, epilepsy, encephalopathy, Huntington's disease, ataxia, dystonia, encephalitis, dysarthria, Alzheimer's disease, autism, or migraines.

In other embodiments, the methods are directed to treating subjects having cancer. Broadly, the compounds of the present invention may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, cerebellar astrocytoma, cerebral astrocytoma/malignant glioma, ependymoma, medulloblastoma, supratentorial primitive neuroectodeimal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), germ cell tumor, ovarian germ cell tumor, gestational trophoblastic tumor glioma, head and neck cancer, Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilms' Tumor, clear cell renal cell carcinoma), liver cancer, lung cancer (e.g., non-small cell lung cancer and small cell lung cancer), Waldenstrom's macroglobulinema, melanoma, intraocular (eye) melanoma, merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, pituitary tumor, plasma cell neoplasm/multiple myeloma, pleuropulmonary blastoma, prostate cancer, retinoblastoma rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, transitional cell cancer of the renal pelvis and ureter and other urinary organs, urethral cancer, gestational trophoblastic tumor, vaginal cancer and vulvar cancer.

Sarcomas that may be treatable with compounds of the present invention include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue) and mesenchymous or mixed mesodermal tumor (mixed connective tissue types).

In some embodiments, methods of the present invention entail treatment of subjects having cell proliferative diseases or disorders of the hematological system, liver (hepatocellular), brain, lung, colorectal (e.g., colon), pancreas, prostate, skin, ovary, breast, skin (e.g., melanoma), and endometrium.

As used herein, “cell proliferative diseases or disorders of the hematologic system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, primary CNS lymphoma (PCNSL), marginal zone lymphoma (MZL), leukemia, including chronic lymphocytic leukemia (CLL), childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the long, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), squamous cell carcinoma, adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell types).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

The bifunctional compounds of formula I and their pharmaceutically acceptable salts or stereoisomers may be administered to a patient, e.g., a cancer patient, as a monotherapy or by way of combination therapy, and as a front-line therapy or a follow-on therapy for patients who are unresponsive to front-line therapy. Therapy may be “first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which have been partially successful but are intolerant to the particular treatment. Therapy may also be given as an adjuvant treatment, i.e., to prevent reoccurrence of cancer in patients with no currently detectable disease or after surgical removal of a tumor. Thus, in some embodiments, the compound may be administered to a patient who has received another therapy, such as chemotherapy, radioimmunotherapy, surgical therapy, immunotherapy, radiation therapy, targeted therapy or any combination thereof.

The methods of the present invention may entail administration of a bifunctional compound of formula I or a pharmaceutical composition thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails a 28-day cycle which includes daily administration for 3 weeks (21 days). In other embodiments, the bifunctional compound may be dosed twice a day (BID) over the course of two and a half days (for a total of 5 doses) or once a day (QD) over the course of two days (for a total of 2 doses). In other embodiments, the bifunctional compound may be dosed once a day (QD) over the course of five days.

### Combination Therapy

The bifunctional compounds of formula I and their pharmaceutically acceptable salts or stereoisomers may be used in combination or concurrently with at least one other active agent, e.g., anti-cancer agent or regimen, in treating diseases and disorders. The terms “in combination” and “concurrently in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a bifunctional compound of formula I in combination with one or more additional therapeutics known for use in treating the disease or condition (e.g., cancer). The dosage of the additional anticancer therapeutic may be the same or even lower than known or recommended doses. See, Hardman et al., eds., Goodman & Gilman's The Pharmacological Basis Of Basis Of Therapeutics, 10th ed., McGraw-Hill, New York, 2001; Physician's Desk Reference 60th ed., 2006. For example, anti-cancer agents that may be used in combination with the inventive compounds are known in the art. See, e.g., U.S. Pat. No. 9,101,622 (Section 5.2 thereof) and U.S. Pat. No. 9,345,705 B2 (Columns 12-18 thereof). Representative examples of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bispecific antibodies) and CAR-T therapy.

In some embodiments, the bifunctional compound of formula I and the additional anticancer therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more anticancer therapeutics may be administered within the same patient visit.

In some embodiments, the bifunctional compound of formula I and the additional agent or therapeutic (e.g., an anti-cancer therapeutic) are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

### Pharmaceutical Kits

The present compositions may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain the bifunctional compound of formula I or a pharmaceutically acceptable salt or stereoisomer or a pharmaceutical composition that contains the bifunctional compound. The kits or pharmaceutical systems of the invention may also include printed instructions for using the compounds and compositions.

These and other aspects of the present invention will be further appreciated upon consideration of the following examples, which are intended to illustrate certain particular embodiments of the invention but are not intended to limit its scope, as defined by the claims.

### EXAMPLES

### Example 1: Synthesis of 3-(3-(((4R)-7-(6-(2-((2-(2,6-dioxopiperidin-3-yl)-1,3-dioxoisoindolin-4-yl)oxy)acetamido)hexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoic Acid (1)

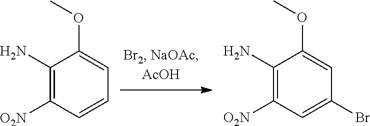

### 4-bromo-2-methoxy-6-nitroaniline

2-methoxy-6-nitrophenylamine (5.0 g, 29.8 mmol) was dissolved in AcOH (70 mL) and NaOAc (2.69 g, 32.8 mmol) was added. Br₂ (2.4 mL, 48 mmol) was then added slowly while stirring at room temperature, and a precipitate formed. After stirring for 1 hour, water was added. The solid was then filtered, and washed with water to yield 4-bromo-2-methoxy-6-nitroaniline (7.2 g, 29.0 mmol, 97%) as an orange solid. LC/MS m/z calculated for [M+H]+ 247.0, found 247.0.

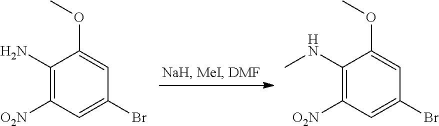

### 4-bromo-2-methoxy-N-methyl-6-nitroaniline

4-bromo-2-methoxy-6-nitroaniline (7.2 g, 29 mmol) was dissolved in DMF (70 mL) and cooled to 0° C. NaH (1.2 g, 30 mmol, 60% dispersion in mineral oil) was then added, and the mixture stirred for 1 hour at 0° C. Iodomethane (2.1 mL, 33 mmol) dissolved in DMF (5 mL) was then added dropwise over about 5 minutes. After stirring for 30 minutes, water was added. The mixture was filtered, solids washed with water, and dried to obtain 4-bromo-2-methoxy-N-methyl-6-nitroaniline (6.0 g, 23 mmol, 79%) as red solid. LC/MS m/z calculated for [M+H]⁺ 261.0, found 262.9.

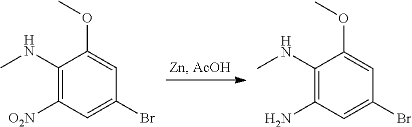

### 4-bromo-6-methoxy-N1-methylbenzene-1,2-diamine

4-bromo-2-methoxy-N-methyl-6-nitroaniline (6.0 g, 23 mmol) was dissolved in AcOH (70 mL). At room temperature, Zn (5.1 g, 78 mmol) was added portionwise over 1 hour, taking care to keep the temperature of the reaction mixture moderate. The reaction was filtered through Celite, and the solids were washed with EtOAc. The filtrate was concentrated to provide 4-bromo-6-methoxy-N1-methylbenzene-1,2-diamine (2.4 g, 10 mmol, 45%). LC/MS m/z calculated for [M+H]⁺ 231.0, found 231.0.

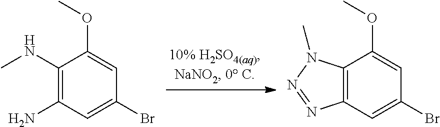

### 5-bromo-7-methoxy-1-methyl-1H-benzo[d][1,2,3]

4-bromo-6-methoxy-N1-methylbenzene-1,2-diamine (7.5 g, 32 mmol) was suspended in 10% H₂SO_{4(aq)} (40 mL) at room temperature. This mixture was then cooled to 0° C. and, while stirring, NaNO₂ (3.13 g, 45.4 mmol) was added portionwise over 30 minutes. During that time, a thick black substance separated from the orange suspension. The mixture was stirred for another 30 minutes, diluted with water, followed by extraction with EtOAc (3×20 mL). Combined extracts were washed with brine, dried with Na₂SO₄, concentrated, and purified by silica gel chromatography to provide the title compound (1.3 g, 5.4 mmol, 17%). LC/MS m/z calculated for [M+H]⁺ 242.0, found 241.9.

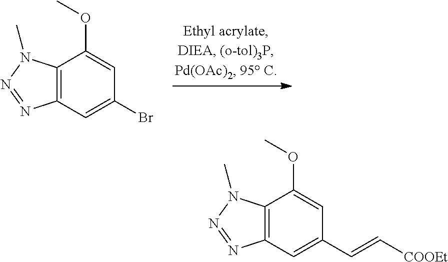 [SMILES: COc1cc(Br)cc2nnn(C)c12 ; CCOC(=O)/C=C/c1cc(OC)c2c(c1)nnn2C]

### Ethyl 3-(7-methoxy-1-methyl-1H-benzo[d][1,2,3]triazol-5-yl)acrylate

5-bromo-7-methoxy-1-methyl-1H-benzo[d][1,2,3]triazole (2.63 g, 10.91 mmol), DIEA (4.75 mL, 27.3 mmol), tri(o-tolyl)phosphine (644 mg, 2.18 mmol), and Pd(OAc)₂ (245 mg, 1.09 mmol) were dissolved in DMF (30 mL). Ethyl acrylate (5.7 mL, 54.5 mmol) was added, the solution was degassed and sparged with N₂, and stirred at 95° C. for 5 h. The reaction mixture was filtered, and water was added followed by extraction with EtOAc (3×15 mL). Combined extracts were washed with brine, dried with Na₂SO₄, concentrated, and purified by silica gel chromatography to provide the title compound (1.33 g, 5.08 mmol, 46%). LC/MS m/z calculated for [M+H]⁺ 262.1, found 262.2.

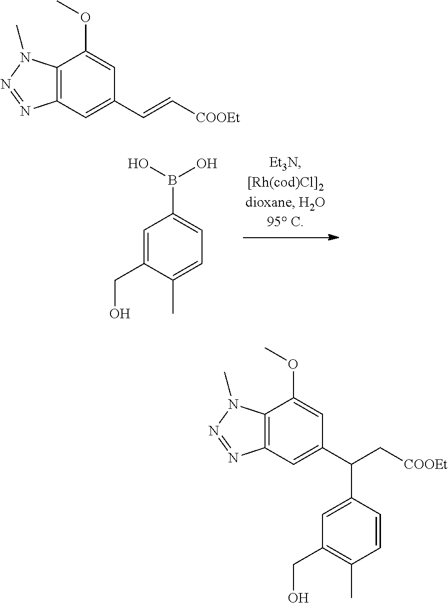

### Ethyl 3-(3-(hydroxymethyl)-4-methylphenyl)-3-(7-methoxy-1-methyl-1H-benzo[d][1,2,3]triazol-5-yl)propanoate

Ethyl (E)-3-(7-methoxy-1-methyl-1H-benzo[d][1,2,3]triazol-5-yl)acrylate (2.33 g, 8.88 mmol), (3-(hydroxymethyl)-4-methylphenyl)boronic acid (2.91 g, 17.5 mmol), triethylamine (2.47 mL, 17.8 mmol), and [Rh(COD)Cl]₂ (438 mg, 0.89 mmol) were dissolved in a mixture of dioxane (40 mL) and water (20 mL). The mixture was degassed and sparged with N₂ twice, then stirred at 95° C. for 5 hours. Water was added and the solution extracted with EtOAc (3×15 mL). Combined extracts were washed with brine, dried with Na₂SO₄, concentrated, and purified by silica gel chromatography to obtain the title compound (2.55 g, 6.65 mmol, 75%) as an orange solid. LC/MS m/z calculated for [M+H]⁺ 384.2, found 384.3.

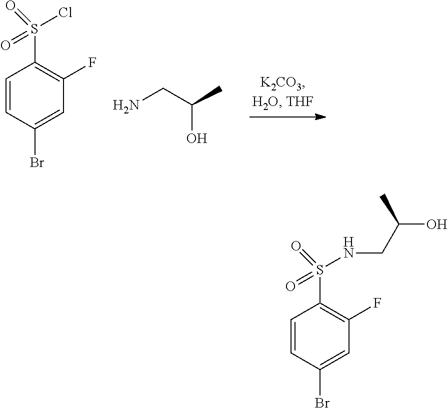

### (R)-4-bromo-2-fluoro-N-(2-hydroxypropyl)

4-bromo-2-fluorobenzenesulfonyl chloride (6.54 g, 24.0 mmol) was dissolved in a mixture of THF (40 mL) and water (10 mL) at room temperature. K₂CO₃ (3.31 g, 24.0 mmol) was added, followed by (R)-1-aminopropan-2-ol (1.87 mL, 1.80 g, 24.0 mmol). The solution was stirred for 24 hours, at which point water was added and the mixture extracted with ethyl acetate (3×20 mL). Combined extracts were washed with brine, dried using Na₂SO₄, and concentrated to obtain the title compound (7.45 g, 23.9 mmol, 99%).

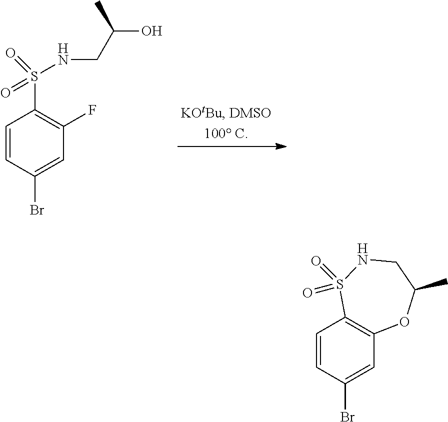 [SMILES: C[C@@H](O)CNS(=O)(=O)c1ccc(Br)cc1F ; C[C@@H]1CNS(=O)(=O)c2ccc(Br)cc2O1]

### (R)-7-bromo-4-methyl-3,4-dihydro-2H-benzo[b][1,4,5]oxathiazepine 1,1-dioxide

(R)-4-bromo-2-fluoro-N-(2-hydroxypropyl)benzenesulfonamide (7.45 g, 23.9 mmol) was dissolved in DMSO (80 mL) and treated with KOtBu (8.03 g, 71.7 mmol) at room temperature. The mixture was placed in an oil bath at 100° C. for 1 hour and then removed and allowed to cool to room temperature. Water was added, and the solution acidified to pH ˜6 using aqueous HCl and extracted with ethyl acetate (3×25 mL). Combined organic extracts were washed with water, brine, dried using Na₂SO₄, and concentrated. The crude oil was then purified by silica gel chromatography to provide (R)-7-bromo-4-methyl-3,4-dihydro-2H-benzo[b][1,4,5]oxathiazepine 1,1-dioxide (5.06 g, 17.3 mmol, 72%).

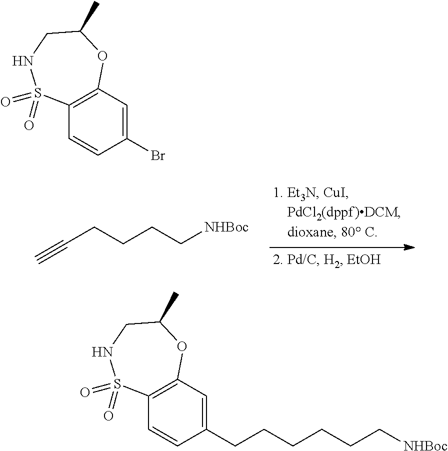 [SMILES: C[C@@H]1CNS(=O)(=O)c2ccc(Br)cc2O1 ; C#CCCCCNC(=O)OC(C)(C)C ; C[C@@H]1CNS(=O)(=O)c2ccc(CCCCCCNC(=O)OC(C)(C)C)cc2O1]

### Tert-Butyl (R)-(6-(4-methyl-1,1-dioxido-3,4-dihydro-2H-benzo[b][1,4,5]oxathiazepin-7-yl)hexyl)carbamate

(R)-7-bromo-4-methyl-3,4-dihydro-2H-benzo[b][1,4,5]oxathiazepine 1,1-dioxide (305 mg, 1.04 mmol), t-butyl hex-5-yn-1-yl carbamate (308 mg, 1.56 mmol), Et₃N (723 μL, 5.20 mmol), CuI, (40 mg, 0.21 mmol) and PdCl₂(dppf).DCM (82 mg, 0.10 mmol) were added to 1,4-dioxane (5 mL). The mixture was degassed and sparged with N₂ and stirred at 80° C. for 4 hours. After filtering through Celite, the solution was diluted with water and extracted with EtOAc (5×10 mL). Combined extracts were concentrated and purified twice by silica gel chromatography to provide tert-butyl (R)-(6-(4-methyl-1,1-dioxido-3,4-dihydro-2H-benzo[b][1,4,5]oxathiazepin-7-yl)hex-5-yn-1-yl)carbamate as an amber oil. LC/MS m z calculated for [M+H]⁺ 409.2, found 409.0.

This oil was then dissolved in EtOH (15 mL), Pd/C was added (50 mg), and the suspension vigorously stirred under H₂ (1 atm) overnight. The suspension was then filtered, and the solvent evaporated to yield the title compound (335 mg, 0.81 mmol, 78% over two steps) as an amber oil. LC/MS m/z calculated for [M+H]⁺ 413.2, found 413.3.

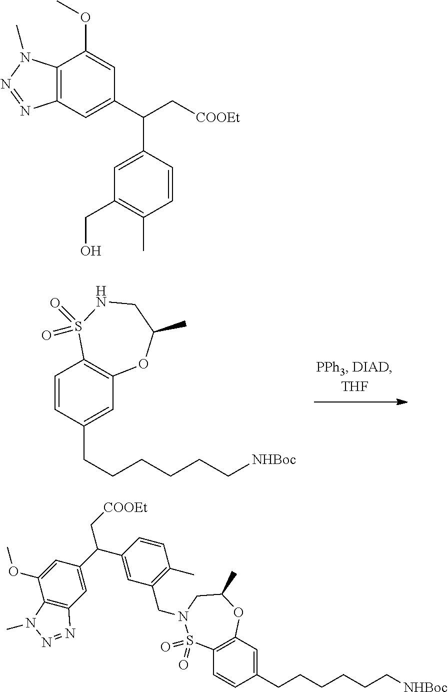

### ethyl 3-(3-(((R)-7-(6-((tert-butoxycarbonyl)amino)hexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoate

Ethyl3-(3-(hydroxymethyl)-4-methylphenyl)-3-(7-methoxy-1 -methyl-1H-benzo[d][1,2,3]triazol-5-yl)propanoate (100 mg, 0.24 mmol), tert-butyl (R)-(6-(4-methyl-1,1-dioxido-3,4-dihydro-2H-benzo[b][1,4,5]oxathiazepin-7-yl)hexyl)carbamate (116 mg, 0.24 mmol), and triphenylphosphine (126 mg, 0.48 mmol) were dissolved in THF (2 mL). DIAD (94 μL, 0.48 mmol) was then added. After 20 minutes, the solvent was evaporated and the residue purified by HPLC to obtain the title compound (117 mg, 0.15 mmol, 63%). LC/MS m/z calculated for [M+H]⁺ 778.4, found 778.3.

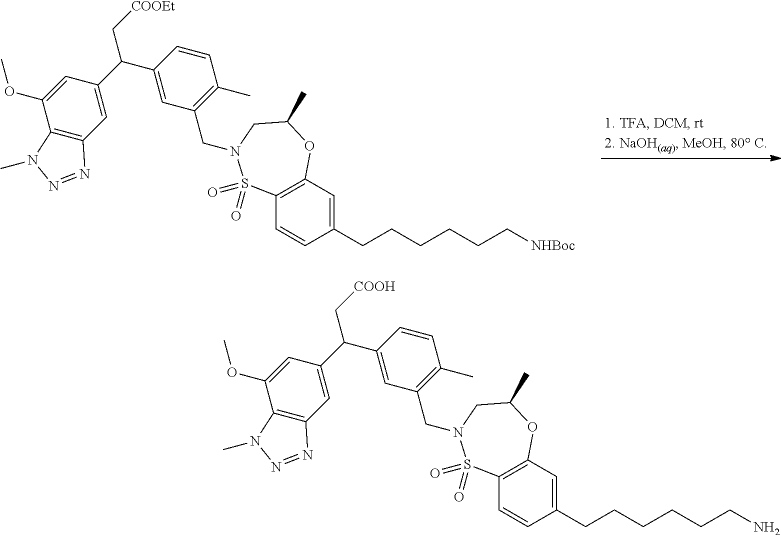

### Ethyl (S)-3-(3-(((R)-7-(6-aminohexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoic Acid

Ethyl 3-(3-(((R)-7-(6-((tert-butoxycarbonyl)amino)hexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoate (65 mg, 0.084 mmol) was dissolved in DCM (1 mL) and TFA (1 mL). The solution was stirred for 1 hour before the solvent was evaporated to obtain the amine as a TFA salt. LC/MS m/z calculated for [M+H]⁺ 678.3, found 678.5.

The residue was then dissolved in MeOH (1 mL). NaOH_{(aq)} (1N, 400 μL) was added and the solution stirred at 80° C. for 2 hours. The mixture was then acidified with HCl_{(aq)}, and the solvent was evaporated. The residue was suspended in EtOH, filtered, and concentrated to provide the title compound (52 mg, 0.069 mmol, 82%). LC/MS m/z calculated for [M+H]⁺ 649.3, found 649.3.

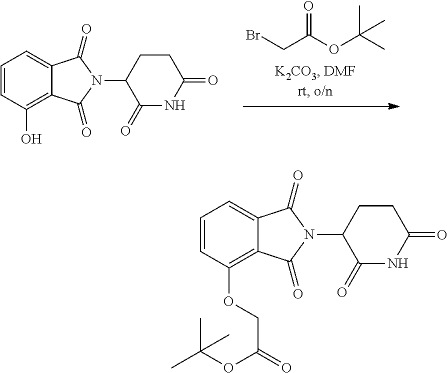

### Tert-Butyl 2-((2-(2,6-dioxopiperidin-3-yl)-1,3-dioxoisoindolin-4-yl)oxy)acetate

T-butyl bromoacetate (199 mg, 1.02 mmol) was added to a mixture of 2-(2,6-dioxopiperidin-3-yl)-4-hydroxyisoindoline-1,3-dione (200 mg, 0.73 mmol) and K₂CO₃ (304 mg, 2.20 mmol) in 3 mL DMF at room temperature. The mixture was stirred overnight and then quenched with water. The aqueous mixture was then extracted with 3×5 mL ethyl acetate, washed with brine, dried using Na₂SO₄, and concentrated. Purification by silica gel chromatography provided tert-butyl 2-((2-(2,6-dioxopiperidin-3-yl)-1,3-dioxoisoindolin-4-yl)oxy)acetate (245 mg, 0.63 mmol, 86%) as a white crystalline solid. LC/MS m/z calculated for [M+2H−tBu]⁺ 333.1, found 333.1.

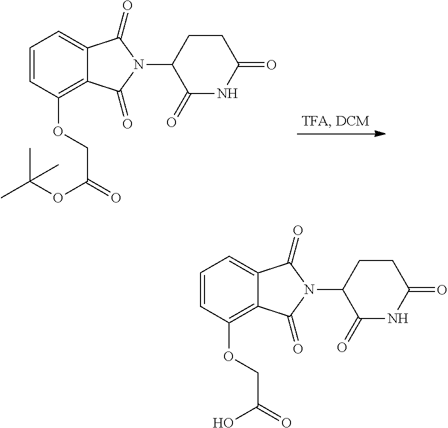 [SMILES: CC(C)(C)OC(=O)COc1cccc2c1C(=O)N(C1CCC(=O)NC1=O)C2=O ; O=C(O)COc1cccc2c1C(=O)N(C1CCC(=O)NC1=O)C2=O]

### 2-((2-(2,6-dioxopiperidin-3-yl)-1,3-dioxoisoindolin-4-yl)oxy)acetic acid

Tert-butyl 2-((2-(2,6-dioxopiperidin-3-yl)-1,3-dioxoisoindolin-4-yl)oxy)acetate (140 mg, 0.036 mmol) was dissolved in 1 mL DCM and 1 mL TFA, and stirred for 1 hour at room temperature. The solvent was evaporated to obtain 2-((2-(2,6-dioxopiperidin-3-yl)-1,3-dioxoisoindolin-4-yl)oxy)acetic acid (121 mg, 0.036 mmol, 101%) as a white solid. LC/MS m/z calculated for [M+H]⁺ 333.1, found 332.7.

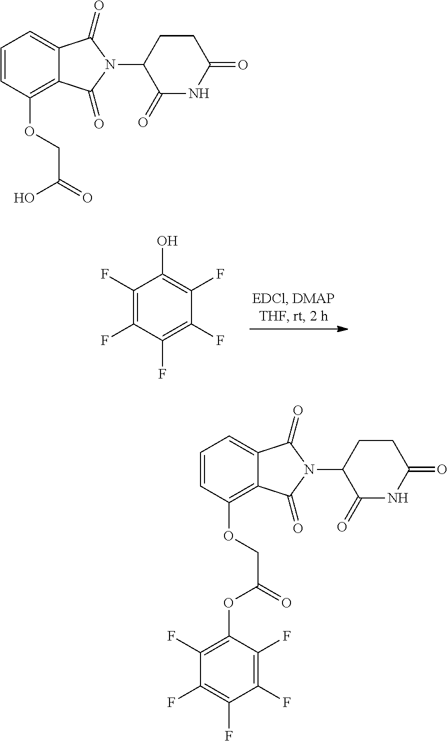 [SMILES: O=C(O)COc1cccc2c1C(=O)N(C1CCC(=O)NC1=O)C2=O ; Oc1c(F)c(F)c(F)c(F)c1F ; O=C1CCC(N2C(=O)c3cccc(OCC(=O)Oc4c(F)c(F)c(F)c(F)c4F)c3C2=O)C(=O)N1]

### Perfluorophenyl 2-((2-(2,6-dioxopiperidin-3-yl)-1,3-dioxoisoindolin-4-yl)oxy)acetate

DMAP (1.0 mg, 0.0082 mmol) and EDCI (5 mg, 0.025 mmol) were added to a solution of 2-((2-(2,6-dioxopiperidin-3-yl)-1,3-dioxoisoindolin-4-yl)oxy)acetic acid (7.7 mg, 0.023 mmol) and 2,3,4,5,6-pentafluorophenol (5 mg, 0.025 mmol) in THF (1 mL) at room temperature. The solution was stirred at room temperature for 2 hours to produce perfluorophenyl 2-((2-(2,6-dioxopiperidin-3-yl)-1,3-dioxoisoindolin-4-yl)oxy)acetate. The solvent was evaporated and the crude product used without purification. LC/MS m/z calculated for [M+H]⁺ 499.1, found 499.3.

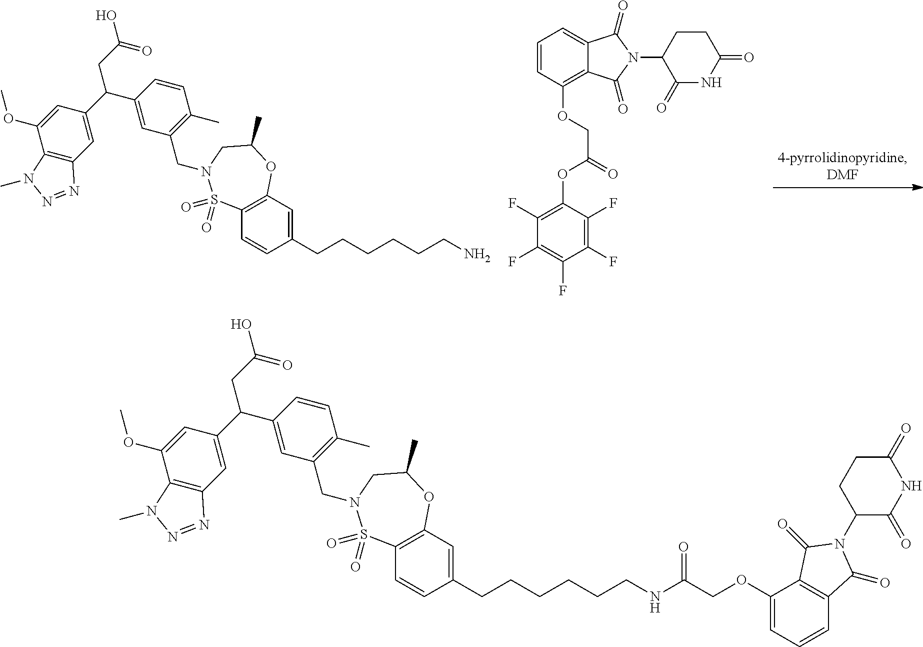 [SMILES: COc1cc(C(CC(=O)O)c2ccc(C)c(CN3C[C@@H](C)Oc4cc(CCCCCCN)ccc4S3(=O)=O)c2)cc2nnn(C)c12 ; O=C1CCC(N2C(=O)c3cccc(OCC(=O)Oc4c(F)c(F)c(F)c(F)c4F)c3C2=O)C(=O)N1 ; COc1cc(C(CC(=O)O)c2ccc(C)c(CN3C[C@@H](C)Oc4cc(CCCCCCNC(=O)COc5cccc6c5C(=O)N(C5CCC(=O)NC5=O)C6=O)ccc4S3(=O)=O)c2)cc2nnn(C)c12]

### 3-(3-(((4R)-7-(6-(2-((2-(2,6-dioxopiperidin-3-yl)-1,3-dioxoisoindolin-4-yl)oxy)acetamido)hexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoic Acid (1)

Perfluorophenyl 2-((2-(2,6-dioxopiperidin-3-yl)-1,3-dioxoisoindolin-4-yl)oxy)acetate (11 mg, 0.023 mmol) was added to a solution of 3-(3-(((R)-7-(6-aminohexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoic acid (15 mg, 0.023 mmol) and 4-pyrrolopyridine (7 mg, 0.046 mmol) in DMF (1 mL). After stirring at room temperature for 1 hour, the reaction mixture was filtered and purified by HPLC to yield the title compound (6.5 mg, 0.0067 mmol, 29%) as a white solid. ¹H NMR (500 MHz, DMSO-d₆) δ 12.13 (s, 1H), 11.12 (s, 1H), 7.95 (t, J=5.7 Hz, 1H), 7.81 (t, J=7.9 Hz, 1H), 7.66 (dd, J=7.9, 2.2 Hz, 1H), 7.50 (d, J=7.3 Hz, 1H), 7.45 (d, J=4.8 Hz, 1H), 7.40 (d, J=8.5 Hz, 1H), 7.36 (s, J=2.4 Hz, 1H), 7.32-7.24 (m, 1H), 7.20-7.15 (m, 1H), 7.15-7.09 (m, 2H), 6.92 (d, J=11.4 Hz, 1H), 5.12 (dd, J=12.9, 5.4 Hz, 1H), 4.78 (s, 2H), 4.51 (m, 1H), 4.46-4.35 (m, 3H), 4.33 (d, J=3.6 Hz, 3H), 3.93 (d, J=5.6 Hz, 3H), 3.80 (d, J=14.0 Hz, 1H), 3.60 (m, 1H), 3.16 (q, J=6.6 Hz, 2H), 3.12-3.06 (m, 2H), 2.89 (m, 1H), 2.77 (dd, J=39.7, 15.0 Hz, 1H), 2.64 (t, J=7.8 Hz, 2H), 2.62-2.54 (m, 1H), 2.24 (d, J=5.1 Hz, 3H), 2.04 (m, 1H), 1.59 (m, 2H), 1.45 (m, 2H), 1.37-1.27 (m, 4H), 1.14 (dd, J=48.8, 6.3 Hz, 3H). LC/MS m z calculated for [M+H]⁺ 964.4, found 964.5.

### Example 2: Synthesis of (3S)-3-(3-(((4R)-7-(6-(2-((2-(2,6-dioxopiperidin-3-yl)-1,3-dioxoisoindolin-4-yl)oxy)acetamido)hexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoic acid (2)

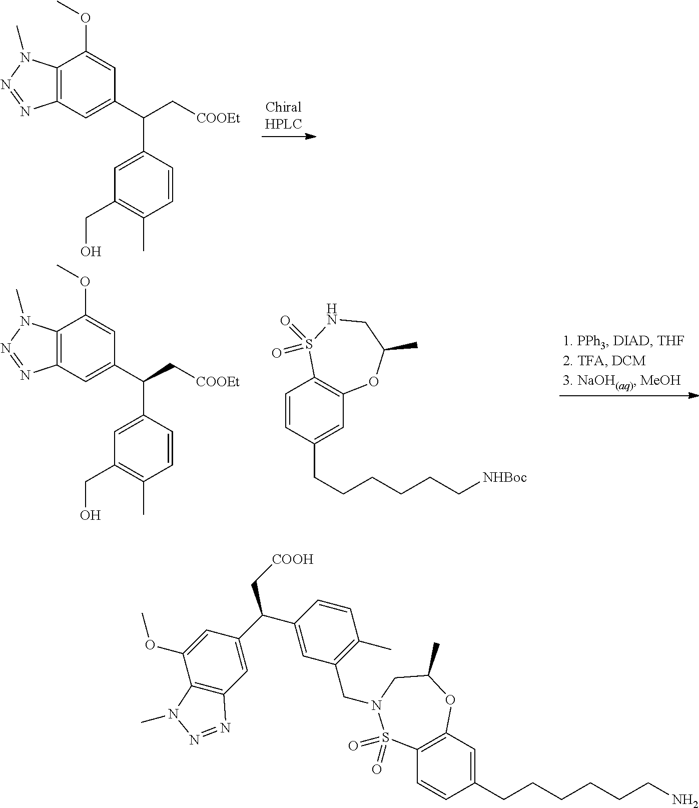

### (S)-3-(3-(((R)-7-(6-aminohexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoic acid

Ethyl 3-(3-(hydroxymethyl)-4-methylphenyl)-3-(7-methoxy-1-methyl-1H-benzo[d][1,2,3]triazol-5-yl)propanoate was purified by chiral HPLC to yield ethyl (S)-3-(3-(hydroxymethyl)-4-methylphenyl)-3-(7-methoxy-1-methyl-TH-benzo[d][1,2,3]triazol-5-yl)propanoate. This intermediate was then carried forward to (S)-3-(3-(((R)-7-(6-aminohexyl)-4-methyl-1,1-dioxido-3,4-dihydro-2H-benzo[b][1,4,5]oxathiazepin-2-yl)methyl)-4-methylphenyl)-3-(7-methoxy-1-methyl-TH-benzo[d][1,2,3]triazol-5-yl)propanoic acid using similar procedures to those outlined above for the racemic mixtures.

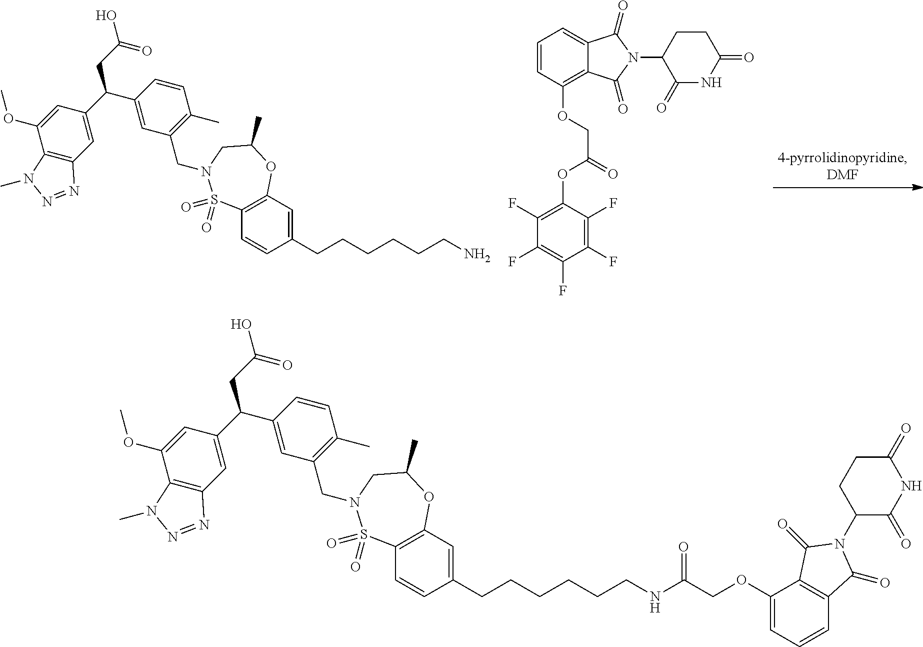 [SMILES: COc1cc([C@@H](CC(=O)O)c2ccc(C)c(CN3C[C@@H](C)Oc4cc(CCCCCCN)ccc4S3(=O)=O)c2)cc2nnn(C)c12 ; O=C1CCC(N2C(=O)c3cccc(OCC(=O)Oc4c(F)c(F)c(F)c(F)c4F)c3C2=O)C(=O)N1 ; COc1cc([C@@H](CC(=O)O)c2ccc(C)c(CN3C[C@@H](C)Oc4cc(CCCCCCNC(=O)COc5cccc6c5C(=O)N(C5CCC(=O)NC5=O)C6=O)ccc4S3(=O)=O)c2)cc2nnn(C)c12]

### (3S)-3-(3-(((4R)-7-(6-(2-((2-(2,6-dioxopiperidin-3-yl)-1,3-dioxoisoindolin-4-yl)oxy)acetamido)hexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoic Acid (2)

Perfluorophenyl 2-((2-(2,6-dioxopiperidin-3-yl)-1,3-dioxoisoindolin-4-yl)oxy)acetate (18.9 mg, 0.038 mmol) was added to a solution of (S)-3-(3-(((R)-7-(6-aminohexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoic acid (26 mg, 0.040 mmol) and 4-pyrrolopyridine (11 mg, 0.070 mmol) in DMF (1 mL). After stirring at room temperature for 1 hour, the reaction mixture was filtered and purified by HPLC to provide the title compound (16.3 mg, 0.017 mmol, 44%) as a white solid. ¹H NMR (500 MHz, DMSO-d₆) δ 12.11 (s, 1H), 11.12 (s, 1H), 7.94 (t, J=5.7 Hz, 1H), 7.81 (dd, J=8.5, 7.3 Hz, 1H), 7.66 (d, J=7.9 Hz, 1H), 7.50 (d, J=7.3 Hz, 1H), 7.45 (s, 1H), 7.40 (d, J=8.5 Hz, 1H), 7.35 (d, J=2.0 Hz, 1H), 7.28 (dd, J=7.8, 1.9 Hz, 1H), 7.17 (dd, J=8.1, 1.6 Hz, 1H), 7.12 (m, 2H), 6.93 (s, 1H), 5.12 (dd, J=12.8, 5.4 Hz, 1H), 4.78 (s, 2H), 4.50 (t, J=8.0 Hz, 1H), 4.41 (d, J=14.0 Hz, 1H), 4.37 (m, 2H), 4.33 (s, 3H), 3.93 (s, 3H), 3.80 (d, J=14.0 Hz, 1H), 3.59 (dd, J=15.4, 10.2 Hz, 1H), 3.16 (m, 2H), 3.09 (m, 2H), 2.89 (ddd, J=17.2, 13.9, 5.4 Hz, 1H), 2.72 (d, J=15.1 Hz, 1H), 2.64 (dd, J=8.7, 6.7 Hz, 2H), 2.57 (m, 1H), 2.23 (s, 3H), 2.04 (m, 1H), 1.59 (m, 2H), 1.45 (m, 2H), 1.32 (m, 4H), 1.09 (d, J=6.3 Hz, 3H). LC/MS m/z calculated for [M+H]⁺ 964.4, found 964.5.

### Example 3: Synthesis of (3S)-3-(7-methoxy-1-methyl-1H-benzo[d][1,2,3]triazol-5-yl)-3-(4-methyl-3-(((4R)-4-methyl-7-(6-(2-((2-(1-methyl-2,6-dioxopiperidin-3-yl)-1,3-dioxoisoindolin-4-yl)oxy)acetamido)hexyl)-1,1-dioxido-3,4-dihydro-2H-benzo[b][1,4,5]oxathiazepin-2-yl)methyl)phenyl)propanoic Acid (3)

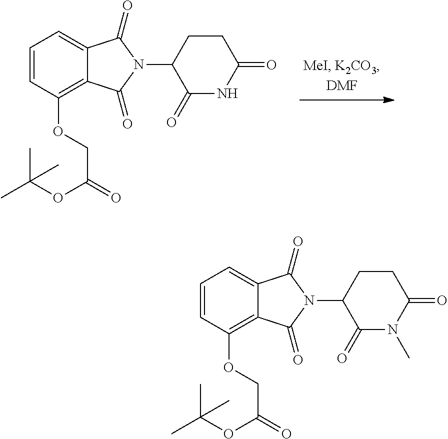 [SMILES: CC(C)(C)OC(=O)COc1cccc2c1C(=O)N(C1CCC(=O)NC1=O)C2=O ; CN1C(=O)CCC(N2C(=O)c3cccc(OCC(=O)OC(C)(C)C)c3C2=O)C1=O]

### Tert-Butyl 2-((2-(1-methyl-2,6-dioxopiperidin-3-yl)-1,3-dioxoisoindolin-4-yl)oxy)acetate

Iodomethane (196 mg, 1.39 mmol) was added to a mixture of tert-butyl 2-((2-(2,6-dioxopiperidin-3-yl)-1,3-dioxoisoindolin-4-yl)oxy)acetate (1008 mg, 0.28 mmol) and K₂CO₃ (77 mg, 0.56 mmol) in 2 mL DMF at room temperature. The reaction was then stirred overnight. Additional iodomethane (196 mg, 1.39 mmol) was added and stirred for another 24 hours. The reaction was then quenched with water, extracted 3×5 ML ethyl acetate, washed with brine, dried using Na₂SO₄, and concentrated. The crude product was purified by silica gel chromatography to provide tert-butyl 2-((2-(1-methyl-2,6-dioxopiperidin-3-yl)-1,3-dioxoisoindolin-4-yl)oxy)acetate (84 mg, 0.21 mmol, 75%). LC/MS m/z calculated for [M+H]⁺ 403.1, [M+H]⁺ found 347.2.

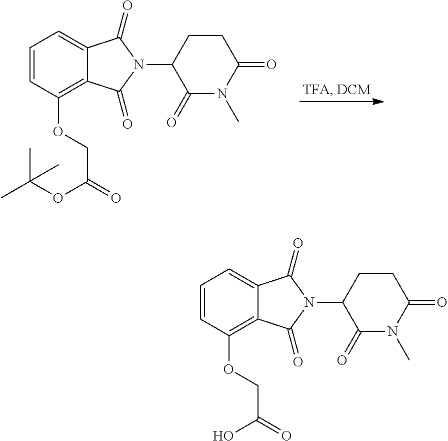 [SMILES: CN1C(=O)CCC(N2C(=O)c3cccc(OCC(=O)OC(C)(C)C)c3C2=O)C1=O ; CN1C(=O)CCC(N2C(=O)c3cccc(OCC(=O)O)c3C2=O)C1=O]

### 2-((2-(1-methyl-2,6-dioxopiperidin-3-yl)-1,3-dioxoisoindolin-4-yl)oxy)acetic acid

Tert-butyl 2-((2-(1-methyl-2,6-dioxopiperidin-3-yl)-1,3-dioxoisoindolin-4-yl)oxy)acetate was dissolved in 1 mL DCM, and 1 mL TFA was added to the solution, which was stirred at room temperature for 1 hour. The solvent was then evaporated and the product lyophilized to yield the title compound (85 mg, 0.19 mmol, 90%). LC/MS m/z calculated for [M+Na]⁺ 349.1, [M+Na]⁺ found 349.2.

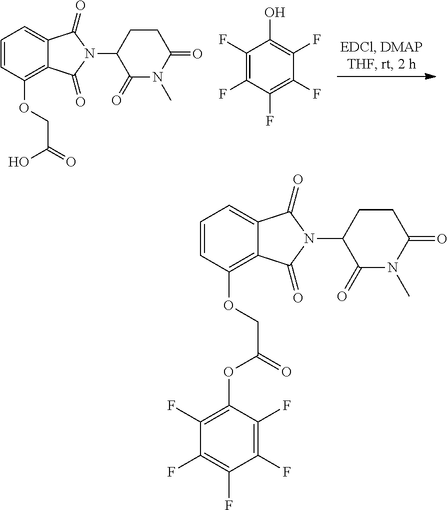

### Perfluorophenyl 2-((2-(1-methyl-2,6-dioxopiperidin-3-yl)-1,3-dioxoisoindolin-4-yl)oxy)acetate

DMAP (1.0 mg, 0.0082 mmol) and EDCI (7.6 mg, 0.040 mmol) were added to a solution of 2-((2-(1-methyl-2,6-dioxopiperidin-3-yl)-1,3-dioxoisoindolin-4-yl)oxy)acetic acid (15 mg, 0.033 mmol) and 2,3,4,5,6-pentafluorophenol (9 mg, 0.050 mmol) in THF (1 mL) at room temperature. The solution was stirred at room temperature for 2 hours to yield perfluorophenyl 2-((2-(1-methyl-2,6-dioxopiperidin-3-yl)-1,3-dioxoisoindolin-4-yl)oxy)acetate. The solvent was evaporated, and the crude product used in the next step without purification.

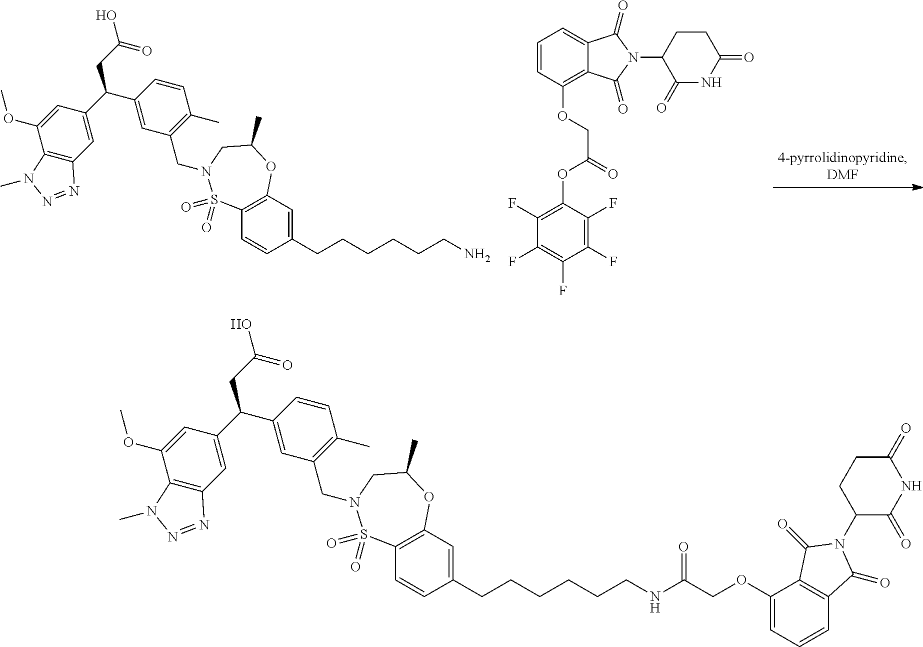 [SMILES: COc1cc([C@@H](CC(=O)O)c2ccc(C)c(CN3C[C@@H](C)Oc4cc(CCCCCCN)ccc4S3(=O)=O)c2)cc2nnn(C)c12 ; O=C1CCC(N2C(=O)c3cccc(OCC(=O)Oc4c(F)c(F)c(F)c(F)c4F)c3C2=O)C(=O)N1 ; COc1cc([C@@H](CC(=O)O)c2ccc(C)c(CN3C[C@@H](C)Oc4cc(CCCCCCNC(=O)COc5cccc6c5C(=O)N(C5CCC(=O)NC5=O)C6=O)ccc4S3(=O)=O)c2)cc2nnn(C)c12]

### (3S)-3-(7-methoxy-1-methyl-1H-benzo[d][1,2,3]triazol-5-yl)-3-(4-methyl-3-(((4R)-4-methyl-7-(6-(2-((2-(1-methyl-2,6-dioxopiperidin-3-yl)-1,3-dioxoisoindolin-4-yl)oxy)acetamido)hexyl)-1,1-dioxido-3,4-dihydro-2H-benzo[b][1,4,5]oxathiazepin-2-yl)methyl)phenyl)propanoic Acid (3)

A solution of perfluorophenyl 2-((2-(1-methyl-2,6-dioxopiperidin-3-yl)-1,3-dioxoisoindolin-4-yl)oxy)acetate (16.9 mg, 0.033) was added to a solution of (S)-3-(3-(((R)-7-(6-aminohexyl)-4-methyl-1,1-dioxido-3,4-dihydro-2H-benzo[b][1,4,5]oxathiazepin-2-yl)methyl)-4-methylphenyl)-3-(7-methoxy-1-methyl-TH-benzo[d][1,2,3]triazol-5-yl)propanoic acid (20 mg, 0.030 mmol) and 4-pyrrolopyridine (9 mg, 0.60 mmol) in 1 mL DMF. The reaction was stirred for 1 hour, filtered, and purified immediately by HPLC to provide the title compound (16.1 mg, 0.016 mmol, 55%) as a white solid. ¹H NMR (500 MHz, DMSO-d₆) δ 12.11 (s, 1H), 7.95 (t, J=5.7 Hz, 1H), 7.82 (dd, J=8.5, 7.3 Hz, 1H), 7.66 (d, J=7.9 Hz, 1H), 7.50 (d, J=7.3 Hz, 1H), 7.45 (s, 1H), 7.41 (d, J=8.5 Hz, 1H), 7.35 (d, J=2.0 Hz, 1H), 7.28 (dd, J=7.8, 1.9 Hz, 1H), 7.17 (dd, J=8.1, 1.6 Hz, 1H), 7.12 (m, 2H), 6.93 (d, J=1.1 Hz, 1H), 5.19 (dd, J=13.0, 5.4 Hz, 1H), 4.78 (s, 2H), 4.50 (t, J=8.0 Hz, 1H), 4.41 (d, J=14.1 Hz, 1H), 4.34 (m, 1H), 4.33 (s, 3H), 3.93 (s, 3H), 3.80 (d, J=14.0 Hz, 1H), 3.58 (m, 1H), 3.16 (m, 2H), 3.09 (m, 2H), 3.02 (s, 3H), 2.95 (m, 1H), 2.75 (m, 2H), 2.64 (dd, J=8.7, 6.7 Hz, 2H), 2.55 (m, 1H), 2.24 (s, 3H), 2.05 (m, 1H), 1.59 (m, 2H), 1.45 (m, 2H), 1.32 (m, 4H), 1.09 (d, J=6.3 Hz, 3H). LC/MS m/z calculated for [M+H]⁺ 978.4, found 978.5.

### Example 4: Synthesis of 3-(3-(((4R)-7-(6-(2-((2-(2,6-dioxopiperidin-3-yl)-1,3-dioxo-2,3-dihydro-1H-benzo[de]isoquinolin-5-yl)oxy)acetamido)hexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoic Acid (4)

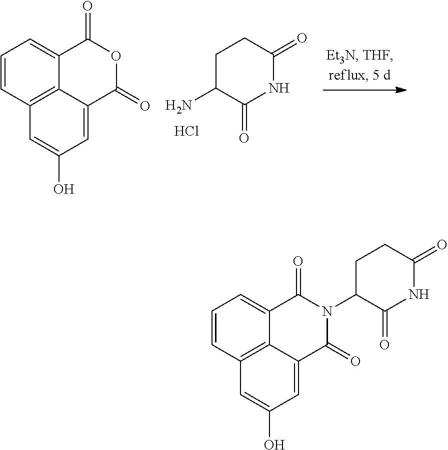

### 2-(2,6-dioxopiperidin-3-yl)-5-hydroxy-1H-benzo[de]isoquinoline-1,3(2H)-dione

3-hydroxy-1,8-naphthalic anhydride (2.14 g, 10.0 mmol) and 3-aminopiperidine-2,6-dione (1.65 g, 10.0 mmol) were dissolved in THF (40 mL) at room temperature, and triethylamine (2.78 mL, 20.0 mmol) was added. The suspension was then refluxed for 5 days, with a green precipitate forming in the first 24 hours and eventually turning black. The solvent was evaporated, water was added, and the mixture was acidified, and stirred for 1 hour. The suspension was then filtered to provide the title compound (3.44 g, 9.53 mmol, 95%) as a green solid. LC/MS m/z calculated for [M+H]⁺ 325.1, found 325.1.

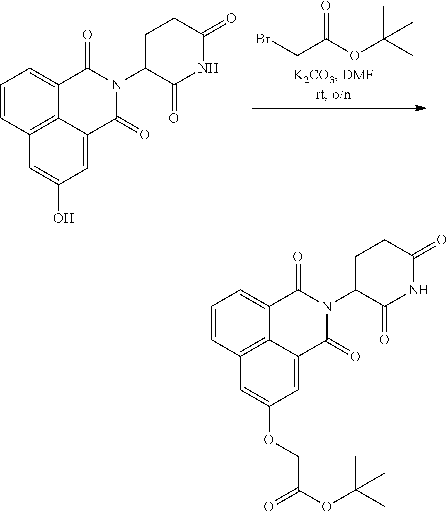

### Tert-Butyl 2-((2-(2,6-dioxopiperidin-3-yl)-1,3-dioxo-2,3-dihydro-1H-benzo[de]isoquinolin-5-yl)oxy)acetate

2-(2,6-dioxopiperidin-3-yl)-5-hydroxy-1H-benzo[de]isoquinoline-1,3(2H)-dione (361 mg, 1.1 mmol) was suspended in 3 mL DMF, followed by addition of K₂CO₃ (276 mg, 2.0 mmol) and t-butyl bromoacetate (234 mg, 1.2 mmol). The blue suspension was stirred at 35° C. for 4 hours, at which point an additional 1.0 mmol of t-butyl bromoacetate was added. After continuing to stir at 35° C. overnight, water was added, and the suspension was filtered to provide the title compound (464 mg, 1.06 mmol, 95%) as a light gray solid. LC/MS m z calculated for [M+2H−tBu]⁺ 383.1, found 383.2.

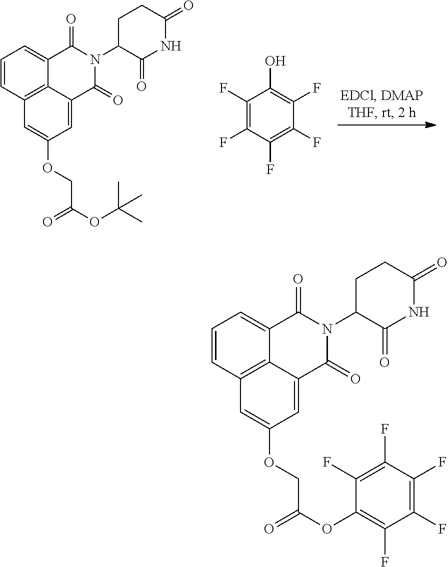 [SMILES: CC(C)(C)OC(=O)COc1cc2c3c(cccc3c1)C(=O)N(C1CCC(=O)NC1=O)C2=O ; Oc1c(F)c(F)c(F)c(F)c1F ; O=C1CCC(N2C(=O)c3cccc4cc(OCC(=O)Oc5c(F)c(F)c(F)c(F)c5F)cc(c34)C2=O)C(=O)N1]

### Perfluorophenyl 2-((2-(2,6-dioxopiperidin-3-yl)-1,3-dioxo-2,3-dihydro-1H-benzo[de]isoquinolin-5-yl)oxy)acetate

DMAP (1.0 mg, 0.0082 mmol) and EDCI (5 mg, 0.025 mmol) were added to a solution of 2-((2-(2,6-dioxopiperidin-3-yl)-1,3-dioxo-2,3-dihydro-1H-benzo[de]isoquinolin-5-yl)oxy)acetic acid (7.7 mg, 0.023 mmol) and 2,3,4,5,6-pentafluorophenol (5 mg, 0.025 mmol) in THF (1 mL) at room temperature. The solution was stirred at room temperature for 2 hours to yield the title compound. The solvent was evaporated and the crude product used without purification. LC/MS m/z calculated for [M+H]⁺ 549.1, found 549.2.

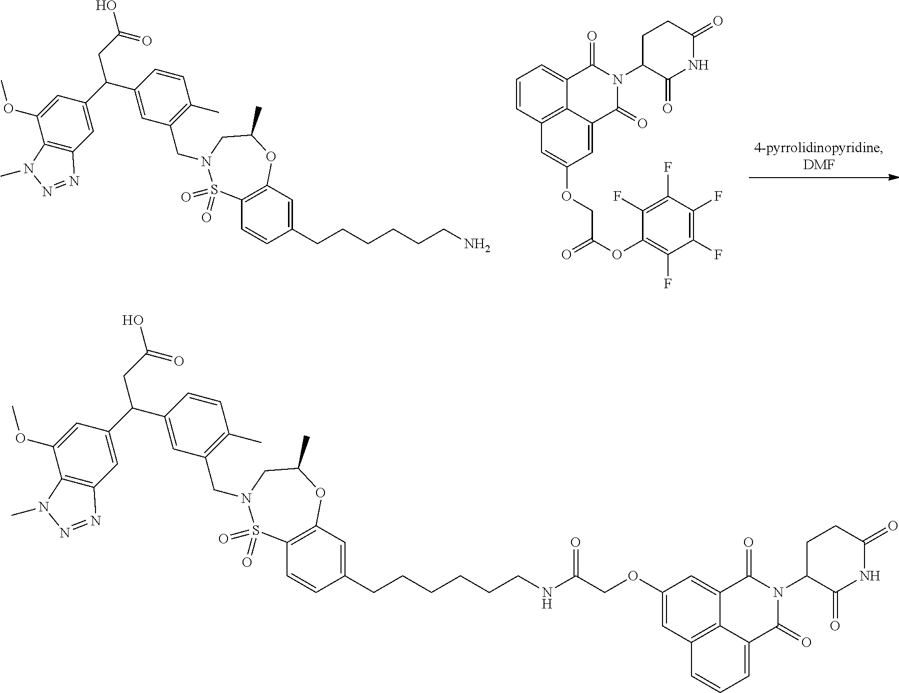 [SMILES: COc1cc(C(CC(=O)O)c2ccc(C)c(CN3C[C@@H](C)Oc4cc(CCCCCCN)ccc4S3(=O)=O)c2)cc2nnn(C)c12 ; O=C1CCC(N2C(=O)c3cccc4cc(OCC(=O)Oc5c(F)c(F)c(F)c(F)c5F)cc(c34)C2=O)C(=O)N1 ; COc1cc(C(CC(=O)O)c2ccc(C)c(CN3C[C@@H](C)Oc4cc(CCCCCCNC(=O)COc5cc6c7c(cccc7c5)C(=O)N(C5CCC(=O)NC5=O)C6=O)ccc4S3(=O)=O)c2)cc2nnn(C)c12]

### 3-(3-(((4R)-7-(6-(2-((2-(2,6-dioxopiperidin-3-yl)-1,3-dioxo-2,3-dihydro-1H-benzo[de]isoquinolin-5-yl)oxy)acetamido)hexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoic Acid (4)

Perfluorophenyl 2-((2-(2,6-dioxopiperidin-3-yl)-1,3-dioxo-2,3-dihydro-1H-benzo[de]isoquinolin-5-yl)oxy)acetate (12.6 mg, 0.023 mmol) was added to a solution of 3-(3-(((R)-7-(6-aminohexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoic acid (15 mg, 0.023 mmol) and 4-pyrrolopyridine (7 mg, 0.046 mmol) in DMF (1 mL). After stirring at room temperature for 1 hour, the reaction mixture was filtered and purified by HPLC to yield the title compound (7.4 mg, 0.0073 mmol, 32%) as a white solid. ¹H NMR (500 MHz, DMSO-d₆) δ 12.12 (s, 1H), 11.02 (s, 1H), 8.31 (m, 3H), 7.96 (dd, J=9.3, 2.6 Hz, 1H), 7.84 (q, J=6.7, 5.9 Hz, 1H), 7.65 (dd, J=8.0, 2.1 Hz, 1H), 7.44 (d, J=4.5 Hz, 1H), 7.36 (s, 1H), 7.27 (m, 1H), 7.12 (m, 3H), 6.91 (d, J=11.2 Hz, 1H), 5.84 (m, 1H), 4.75 (s, 2H), 4.51 (m, 1H), 4.41 (d, J=14.0 Hz, 2H), 4.36 (m, 1H), 4.33 (d, J=3.9 Hz, 3H), 3.92 (d, J=5.7 Hz, 3H), 3.79 (d, J=14.0 Hz, 1H), 3.59 (m, 1H), 3.16 (m, 2H), 3.10 (t, J=7.1 Hz, 2H), 2.94 (m, 1H), 2.77 (dd, J=40.1, 15.1 Hz, 1H), 2.60 (m, 3H), 2.24 (d, J=5.1 Hz, 3H), 2.08 (s, 1H), 2.04 (dd, J=10.4, 5.3 Hz, 1H), 1.49 (m, 4H), 1.26 (m, 4H), 1.14 (dd, J=49.6, 6.2 Hz, 3H). LC/MS m/z calculated for [M+H]⁺ 1014.4, found 1014.5.

### Example 5: Synthesis of (3S)-3-(3-(((4R)-7-(6-(2-((2-(2,6-dioxopiperidin-3-yl)-1,3-dioxo-2,3-dihydro-1H-benzo[de]isoquinolin-5-yl)oxy)acetamido)hexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoic Acid (5)

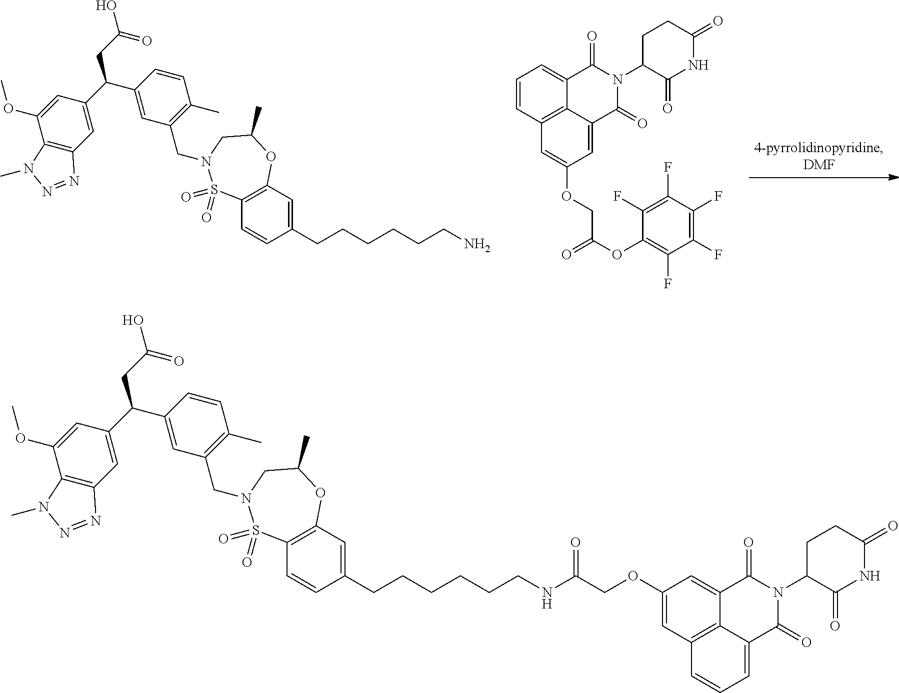 [SMILES: COc1cc([C@@H](CC(=O)O)c2ccc(C)c(CN3C[C@@H](C)Oc4cc(CCCCCCN)ccc4S3(=O)=O)c2)cc2nnn(C)c12 ; O=C1CCC(N2C(=O)c3cccc4cc(OCC(=O)Oc5c(F)c(F)c(F)c(F)c5F)cc(c34)C2=O)C(=O)N1 ; COc1cc([C@@H](CC(=O)O)c2ccc(C)c(CN3C[C@@H](C)Oc4cc(CCCCCCNC(=O)COc5cc6c7c(cccc7c5)C(=O)N(C5CCC(=O)NC5=O)C6=O)ccc4S3(=O)=O)c2)cc2nnn(C)c12]

### (3S)-3-(3-(((4R)-7-(6-(2-((2-(2,6-dioxopiperidin-3-yl)-1,3-dioxo-2,3-dihydro-1H-benzo[de]isoquinolin-5-yl)oxy)acetamido)hexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoic Acid (5)

Perfluorophenyl 2-((2-(2,6-dioxopiperidin-3-yl)-1,3-dioxo-2,3-dihydro-1H-benzo[de]isoquinolin-5-yl)oxy)acetate (24.1 mg, 0.044 mmol) was added to a solution of (S)-3-(3-(((R)-7-(6-aminohexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoic acid (26 mg, 0.040 mmol) and 4-pyrrolopyridine (12 mg, 0.080 mmol) in DMF (1 mL). The reaction was stirred at room temperature for 1 hour, filtered, and immediately purified by HPLC to provide the title compound as a white solid (15.1 mg, 0.015 mmol, 38%). ¹H NMR (500 MHz, DMSO-d₆) δ 12.11 (s, 1H), 11.02 (d, J=2.4 Hz, 1H), 8.31 (m, 3H), 7.95 (dd, J=9.3, 2.6 Hz, 1H), 7.85 (m, 1H), 7.65 (m, 1H), 7.45 (d, J=2.4 Hz, 1H), 7.35 (d, J=1.9 Hz, 1H), 7.28 (dd, J=7.7, 1.9 Hz, 1H), 7.11 (m, 3H), 6.92 (d, J=2.8 Hz, 1H), 5.84 (m, 1H), 4.75 (s, 2H), 4.50 (t, J=8.0 Hz, 1H), 4.41 (d, J=14.0 Hz, 2H), 4.37 (d, J=6.6 Hz, 1H), 4.33 (d, J=4.0 Hz, 3H), 3.93 (s, 3H), 3.79 (d, J=14.0 Hz, 1H), 3.59 (dd, J=15.4, 10.2 Hz, 1H), 3.15 (dt, J=13.9, 7.1 Hz, 2H), 3.08 (dt, J=16.0, 7.9 Hz, 2H), 2.94 (m, 1H), 2.72 (d, J=15.1 Hz, 1H), 2.60 (m, 3H), 2.23 (s, 4H), 2.08 (s, 1H), 2.04 (m, 1H), 1.49 (m, 2H), 1.31 (d, J=6.2 Hz, 1H), 1.26 (m, 4H), 1.09 (d, J=6.2 Hz, 3H). LC/MS m/z calculated for [M+H]⁺ 1014.4, found 1014.5.

### Example 6: Synthesis of (3S)-3-(7-methoxy-1-methyl-1H-benzo[d][1,2,3]triazol-5-yl)-3-(4-methyl-3-(((4R)-4-methyl-7-(6-(2-((2-(1-methyl-2,6-dioxopiperidin-3-yl)-1,3-dioxo-2,3-dihydro-1H-benzo[de]isoquinolin-5-yl)oxy)acetamido)hexyl)-1,1-dioxido-3,4-dihydro-2H-benzo[b][1,4,5]oxathiazepin-2-yl)methyl)phenyl)propanoic Acid (6)

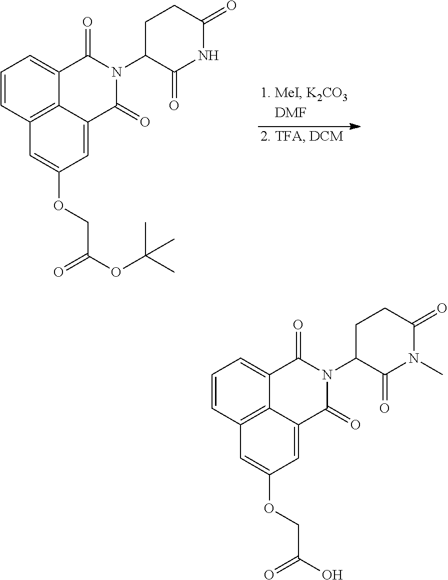 [SMILES: CC(C)(C)OC(=O)COc1cc2c3c(cccc3c1)C(=O)N(C1CCC(=O)NC1=O)C2=O ; CN1C(=O)CCC(N2C(=O)c3cccc4cc(OCC(=O)O)cc(c34)C2=O)C1=O]

### 2-((2-(1-methyl-2,6-dioxopiperidin-3-yl)-1,3-dioxo-2,3-dihydro-1H-benzo[de]isoquinolin-5-yl)oxy)acetic Acid

Tert-butyl 2-((2-(2,6-dioxopiperidin-3-yl)-1,3-dioxo-2,3-dihydro-1H-benzo[de]isoquinolin-5-yl)oxy)acetate (86 mg, 0.20 mmol) and K₂CO₃ (54 mg, 0.39 mmol) were suspended in 2 mL DMF, and iodomethane (139 mg, 0.98 mmol) was added at room temperature. After stirring overnight, water was added and the aqueous mixture was extracted with 3×5 mL DCM. Combined extracts were washed with brine, dried using Na₂SO₄, and concentrated to provide tert-butyl 2-((2-(1-methyl-2,6-dioxopiperidin-3-yl)-1,3-dioxo-2,3-dihydro-1H-benzo[de]isoquinolin-5-yl)oxy)acetate (74 mg, 0.16 mmol, 84%). LC/MS m z calculated for [M+Na]⁺ 475.1, found 475.1.

This ester was then dissolved in 2 mL DCM and 1 mL TFA was added. After the hydrolysis was complete, the solvent was evaporated and the crude residue was purified by silica gel chromatography to obtain the title compound (46.3 mg, 0.12 mmol, 75%). LC/MS m/z calculated for [M+H]⁺ 397.1, found 397.3.

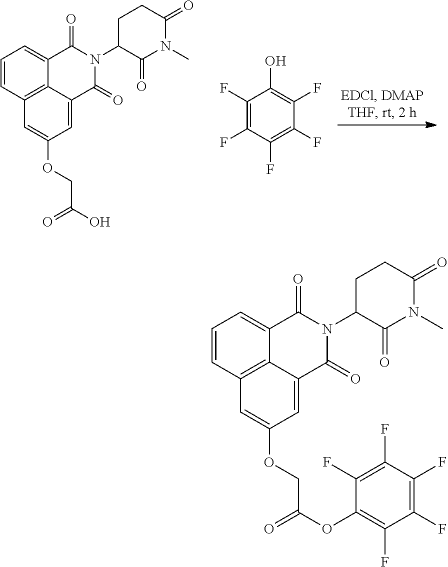 [SMILES: CN1C(=O)CCC(N2C(=O)c3cccc4cc(OCC(=O)O)cc(c34)C2=O)C1=O ; Oc1c(F)c(F)c(F)c(F)c1F ; CN1C(=O)CCC(N2C(=O)c3cccc4cc(OCC(=O)Oc5c(F)c(F)c(F)c(F)c5F)cc(c34)C2=O)C1=O]

### Perfluorophenyl 2-((2-methyl-2,6-dioxopiperidin-3-yl)-1,3-dioxo-2,3-dihydro-1H-benzo[de]isoquinolin-5-yl)oxy)acetate

DMAP (1.5 mg, 0.012 mmol) and EDCI (11 mg, 0.057 mmol) were added to a solution of 2-((2-(1-methyl-2,6-dioxopiperidin-3-yl)-1,3-dioxo-2,3-dihydro-1H-benzo[de]isoquinolin-5-yl)oxy)acetic acid (10 mg, 0.025 mmol) and 2,3,4,5,6-pentafluorophenol (9.0 mg, 0.049 mmol) in THF (1 mL) at room temperature. The solution was stirred at room temperature for 1.5 hours to produce perfluorophenyl 2-((2-(1-methyl-2,6-dioxopiperidin-3-yl)-1,3-dioxo-2,3-dihydro-1H-benzo[de]isoquinolin-5-yl)oxy)acetate. The solvent was evaporated, and the crude product used without purification in the next step.

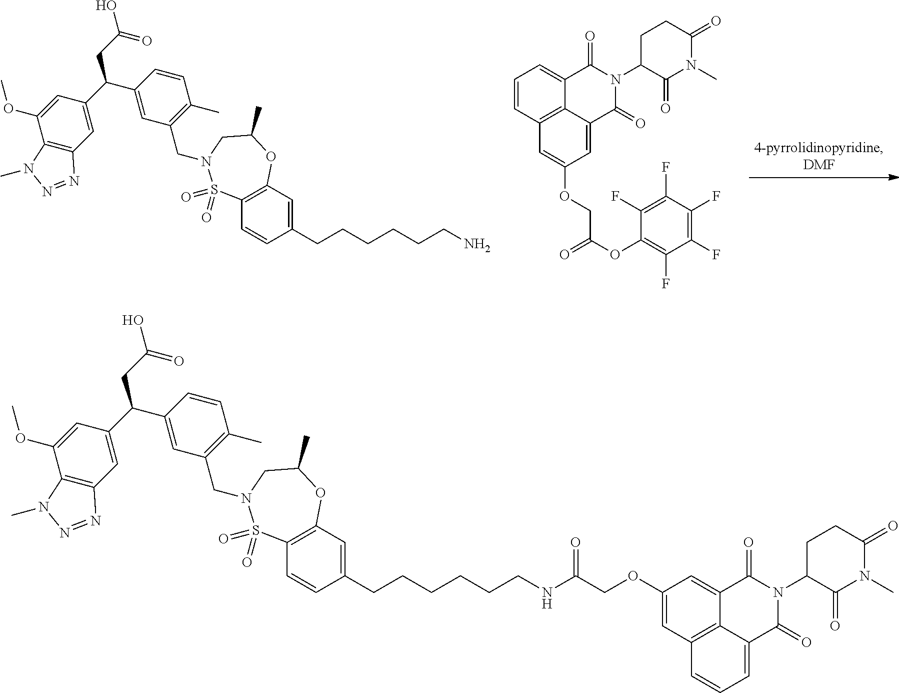

### (3S)-3-(7-methoxy-1-methyl-1H-benzo[d][1,2,3]triazol-5-yl)-3-(4-methyl-3-(((4R)-4-methyl-7-(6-(2-((2-(1-methyl-2,6-dioxopiperidin-3-yl)-1,3-dioxo-2,3-dihydro-1H-benzo[de]isoquinolin-5-yl)oxy)acetamido)hexyl)-1,1-dioxido-3,4-dihydro-2H-benzo[b][1,4,5]oxathiazepin-2-yl)methyl)phenyl)propanoic acid (6)

Perfluorophenyl 2-((2-(1-methyl-2,6-dioxopiperidin-3-yl)-1,3-dioxo-2,3-dihydro-1H-benzo[de]isoquinolin-5-yl)oxy)acetate (14.0 mg, 0.025 mmol) was added to a solution of (S)-3-(3-(((R)-7-(6-aminohexyl)-4-methyl-1,1-dioxido-3,4-dihydro-2H-benzo[b][1,4,5]oxathiazepin-2-yl)methyl)-4-methylphenyl)-3-(7-methoxy-1-methyl-1H-benzo[d][1,2,3]triazol-5-yl)propanoic acid (15 mg, 0.023 mmol) and 4-pyrrolopyridine (8.0 mg, 0.054 mmol) in DMF (1 mL). After stirring at room temperature for 20 min, the reaction mixture was filtered and purified by HPLC to provide the title compound (13.6 mg, 0.013 mmol, 58%) as a white solid. ¹H NMR (500 MHz, DMSO-d₆) δ 12.10 (s, 1H), 8.31 (m, 3H), 7.96 (m, 1H), 7.84 (d, J=8.5 Hz, 1H), 7.65 (d, J=7.9 Hz, 1H), 7.45 (s, 1H), 7.35 (d, J=2.1 Hz, 1H), 7.28 (m, 1H), 7.11 (t, J=7.4 Hz, 3H), 6.92 (d, J=1.1 Hz, 1H), 5.93 (dd, J=12.2, 5.8 Hz, 1H), 4.75 (s, 2H), 4.50 (t, J=8.0 Hz, 1H), 4.41 (d, J=14.0 Hz, 1H), 4.36 (d, J=4.5 Hz, 1H), 4.33 (s, 3H), 3.93 (s, 3H), 3.79 (d, J=14.0 Hz, 1H), 3.59 (dd, J=15.4, 10.2 Hz, 1H), 3.15 (m, 2H), 3.09 (t, J=7.8 Hz, 1H), 3.05 (d, J=2.9 Hz, 3H), 2.99 (m, 1H), 2.75 (m, 2H), 2.58 (m, 4H), 2.23 (s, 3H), 2.08 (s, 1H), 2.05 (m, 1H), 1.47 (m, 4H), 1.28 (m, 4H), 1.09 (d, J=6.3 Hz, 3H). LC/MS m/z calculated for [M+H]⁺ 1028.4, found 1028.4.

### Example 7: Knockdown of KEAP1 in multiple myeloma (MM). 1s Cells

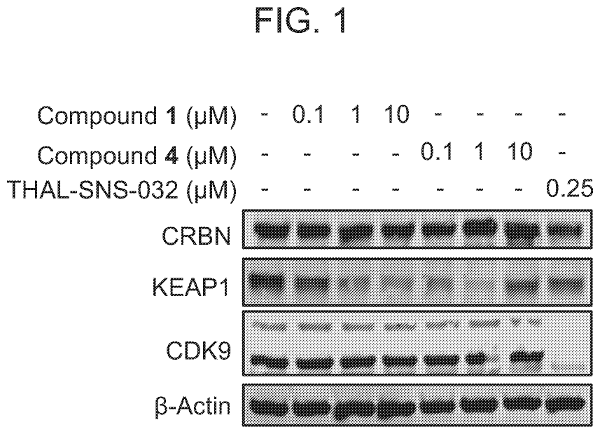

MM.1S cells were treated with 0, 0.1 μM, 1 μM, or 10 μM of compound 1 or compound 4 or 0.25 μM THAL-SNS-032 (a known CDK9 degrader, as a positive control for CDK9 degradation) for 16 hours. Cells were then lysed in RIPA buffer (Sigma) containing protease/phosphatase inhibitor cocktail (Roche). The protein concentrations were measured by bicinchoninic acid assay (BCA) analysis (Pierce™). Equal amounts of protein were resolved by 4-12% Tris-Base gels (Invitrogen), and then transferred to the Immuno-Blot polyvinylidene difluoride (PVDF) membrane (BioRad), and immunoblotted with primary antibodies against KEAP1 (cell signaling), CRBN (cell signaling), CDK9 (cell signaling) and R-Actin (cell signaling), and then immunoblotted with IRDye® 800-labeled goat anti-rabbit IgG and IRDye®800-labeled goat anti-mouse IgG (LI-COR) secondary antibodies. The membranes were detected on Odyssey CLx system. The results, illustrated in FIG. 1, indicated that compound 1 and compound 4 induced the degradation of KEAP1 after 16 hours at the indicated concentrations. THAL-SNS-032 induced CDK9 degradation as expected.

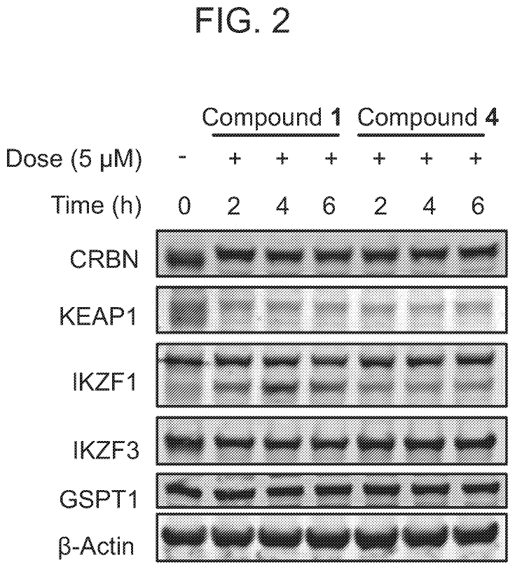

MM.1S cells were treated with 0 or 5 μM compound 1 or compound 4 for 0, 2, 4, or 6 h. Cells were lysed and immunoblotted as described above with antibodies to KEAP1 (cell signaling), CRBN (cell signaling), IKZF1 (cell signaling), IKZF3 (cell signaling), GSPT1 (abcam) and R-Actin (cell signaling). The results, illustrated in FIG. 2, showed that compound 1 and compound 4 induced KEAP1 degradation within 2 h and persisted for at least 6 h at the concentrations of 5 μM, and did not induce the degradation of potential off targets IKZF1, IKZF3 and GSPT1.

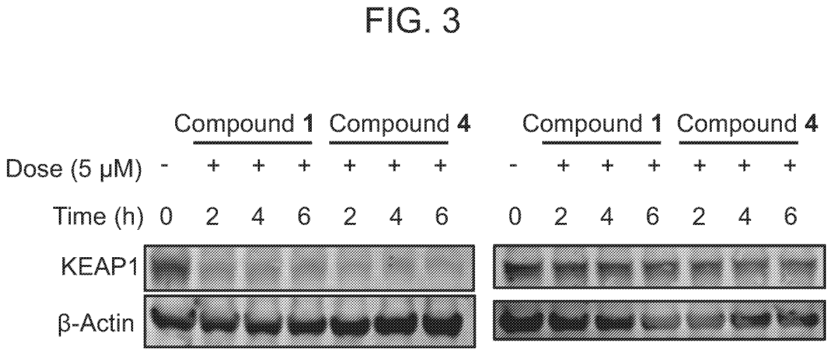

Wild-type (WT) (left panel) or CRBN−/− (right panel) MM.1 S cells were treated with 0 or 5 μM compound 1 or compound 4 for 0, 2, 4, or 6 h. Cells were lysed and immunoblotted as described above with antibodies to KEAP1 (cell signaling) and R-Actin (cell signaling). The results, illustrated in FIG. 3, showed that compound 1 and compound 4 induced KEAP1 degradation in WT MM.1S cells and did not induce KEAP1 degradation in CRBN−/− MM.1S cells, indicating that the KEAP1 degradation is CRBN dependent.

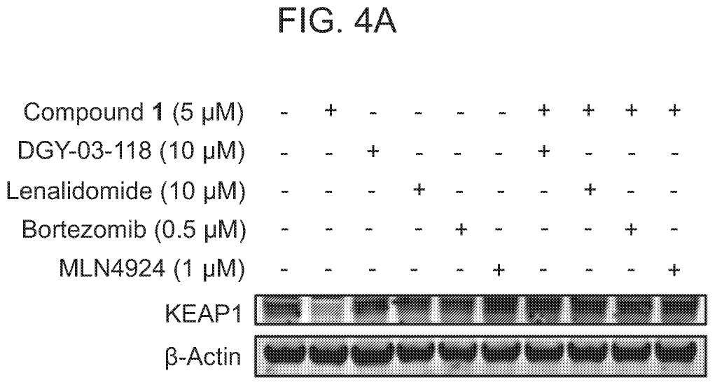

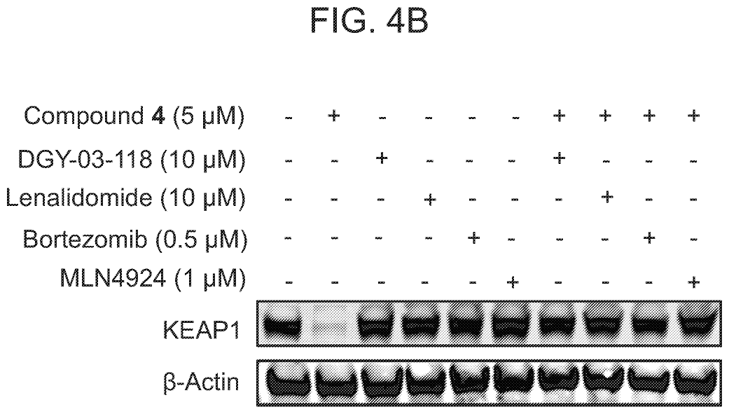

MM.1S cells were pretreated with 10 μM DGY-03-118 (the parental compound and known KEAP1 inhibitor), 10 μM Lenalidomide (CRBN ligand), 0.5 μM Bortezomib (a proteasome inhibitor) and 1 μM MLN4924 (a neddylation inhibitor) for 2 h, and then treated with 5 μM compound 1 for 2 h. Cells were lysed and immunoblotted as described above with antibodies to KEAP1 and β-Actin. The results, illustrated in FIG. 4A, showed that DGY-03-118, Lenalidomide, Bortezomib and MLN4924 rescued the KEAP1 degradation inducing by compound 1, indicating that the KEAP1 degradation are both ligand and proteasome dependent.

MM.1S cells were pretreated with 10 μM DGY-03-118 (the parental compound and known KEAP1 inhibitor), 10 μM Lenalidomide (CRBN ligand), 0.5 μM Bortezomib (a proteasome inhibitor) and 1 μM MLN4924 (a neddylation inhibitor) for 2 h, and then treated with 5 μM compound 4 for 2 h. Cells were lysed and immunoblotted as described above with antibodies to KEAP1 and β-Actin. The results, illustrated in FIG. 4B, showed that DGY-03-118, Lenalidomide, Bortezomib and MLN4924 rescued the KEAP1 degradation inducing by compound 4, indicating that the KEAP1 degradation are both ligand and proteasome dependent.

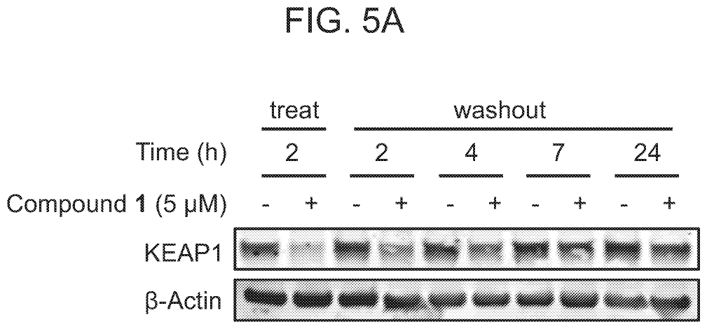

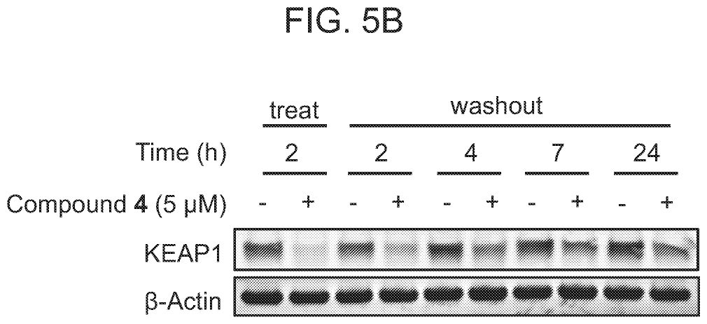

MM.1S cells were treated with 0 or 5 μM compound 1 for 2 h, followed by washout at 2, 4, 7, and 24 hours. Cells were lysed and immunoblotted as described above with antibodies to KEAP1 and β-Actin. The results, illustrated in FIG. 5A, showed that KEAP1 levels in compound 1-treated cells remained below levels in vehicle-treated cells 4 h after washout.

MM.1S cells were treated with 0 or 5 μM compound 4 for 2 h, followed by washout at 2, 4, 7, and 24 hours. Cells were lysed and immunoblotted as described above with antibodies to KEAP1 and β-Actin. The results, illustrated in FIG. 5B, showed that KEAP1 levels in compound 4-treated cells remained below levels in vehicle-treated cells even 24 h after washout, indicating that compound 4 induces sustained depletion of KEAP1.

All patent publications and non-patent publications are indicative of the level of skill of those skilled in the art to which this invention pertains. All these publications (including any specific portions thereof that are referenced) are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A bifunctional compound having a structure represented by formula: 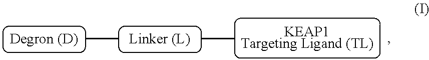
wherein the degron represents a moiety that binds an E3 ubiquitin ligase, and the linker represents a moiety that covalently connects the degron and the targeting ligand, or a pharmaceutically acceptable salt or stereoisomer thereof,
wherein the KEAP1 targeting ligand is represented by formula TL1a or TL1b: 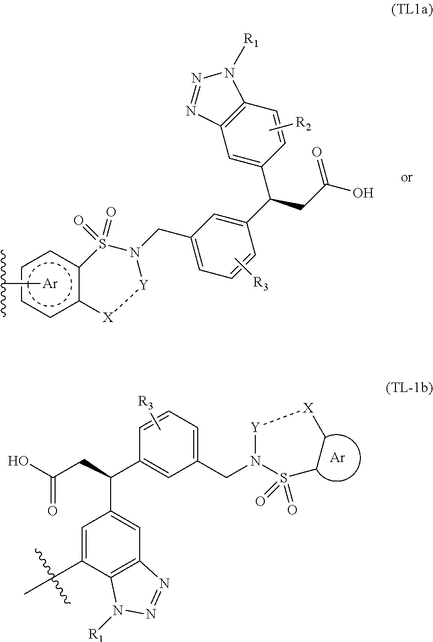
wherein:
R₁ represents optionally substituted C1-C3 alkyl;
R₂ represents Me, OMe, or halo;
R₃ represents Me, OMe, or halo;
X represents H and Y represents optionally substituted C1-C3 alkyl, or
X represents O and Y represents optionally substituted CH₂—CH₂ and X and Y together with the atoms to which they are bound form a 7-membered cyclic sulfonamide;
Ar represents a benzene or pyridine ring, or
wherein the KEAP1 targeting ligand is represented by formula (TL2): 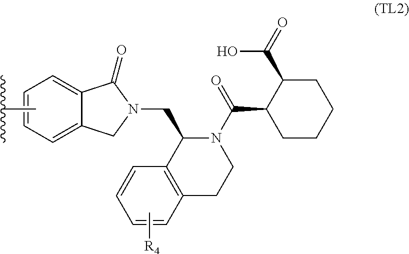
wherein:
R₄ represents optionally substituted C1-C3 alkyl, OMe, or halo, or
wherein the KEAP1 targeting ligand is represented by formula (TL3): 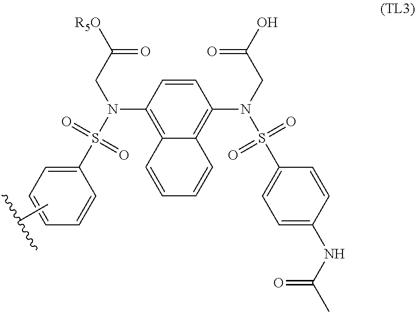
wherein:
R₅ represents optionally substituted C1-C3 alkyl.

2. (canceled)

3. The bifunctional compound of claim 1, wherein the bifunctional compound has a structure represented by: 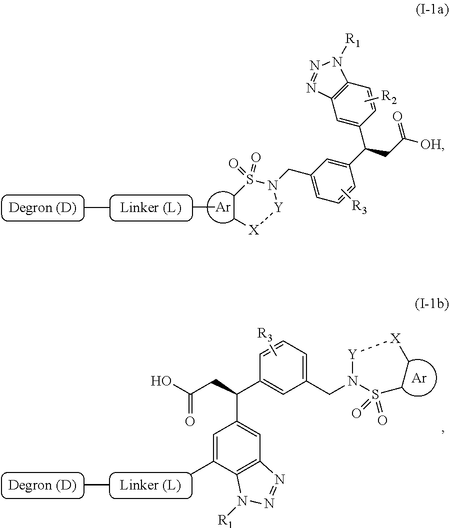
or a pharmaceutically acceptable salt or stereoisomer thereof,
wherein:
R₁ represents optionally substituted C1-C3 alkyl;
R₂ represents Me, OMe, or halo;
R₃ represents Me, OMe, or halo;
X represents H and Y represents optionally substituted C1-C3 alkyl, or X represents —O—, Y represents optionally substituted —CH₂—CH₂— and X and Y together with the atoms to which they are bound form a 7-membered cyclic sulfonamide; and
Ar represents phenyl or pyridinyl.

4. The bifunctional compound of claim 3, wherein the bifunctional compound has a structure represented by: 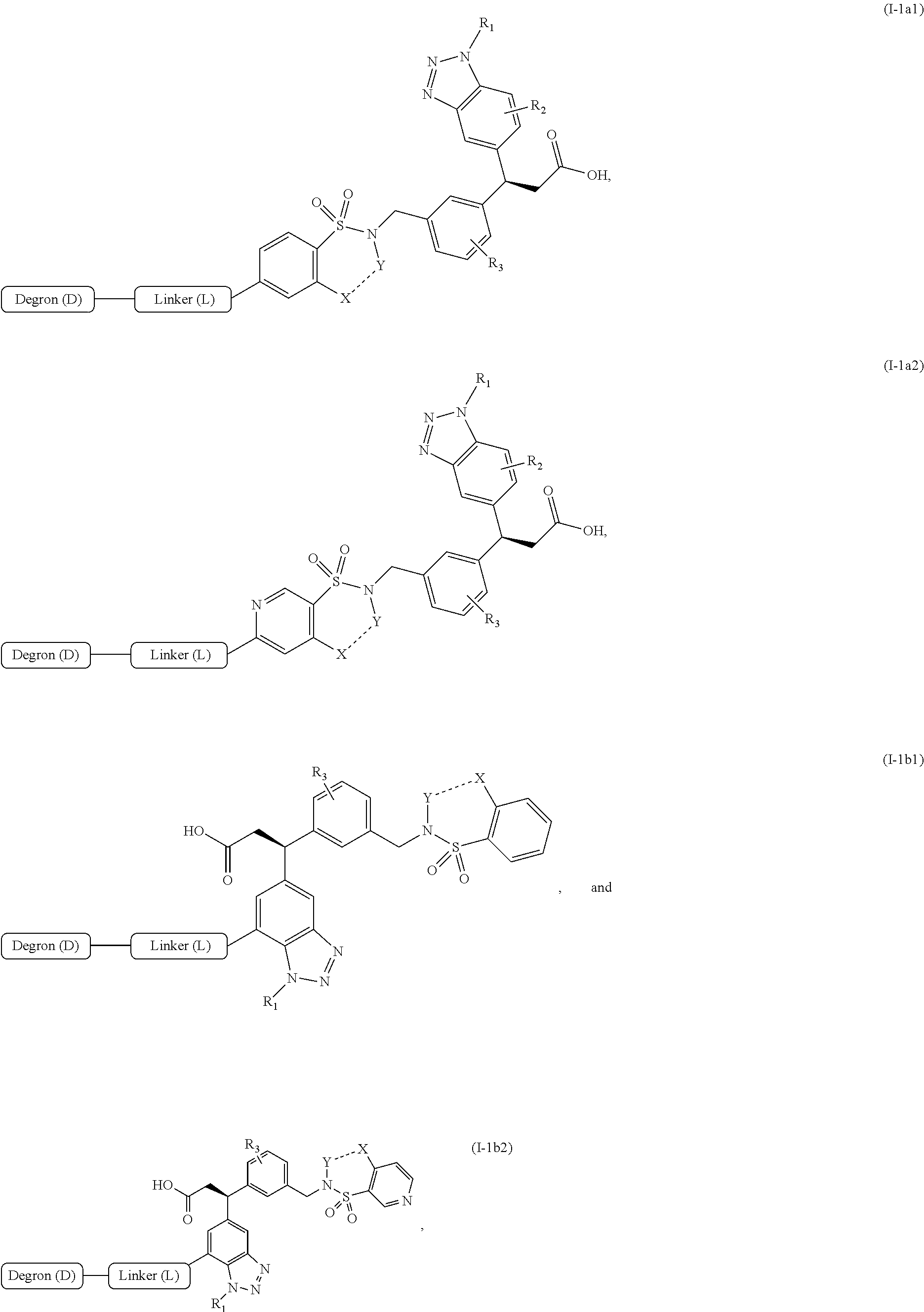
or a pharmaceutically acceptable salt or stereoisomer thereof,
wherein:
R₁ represents optionally substituted C1-C3 alkyl;
R₂ represents Me, OMe, or halo;
R₃ represents Me, OMe, or halo;
X represents H and Y represents optionally substituted C1-C3 alkyl, or X represents —O—, Y represents optionally substituted —CH₂—CH₂— and X and Y together with the atoms to which they are bound form a 7-membered cyclic sulfonamide.

5. (canceled)

6. The bifunctional compound of claim 1, wherein the bifunctional compound has a structure represented by: 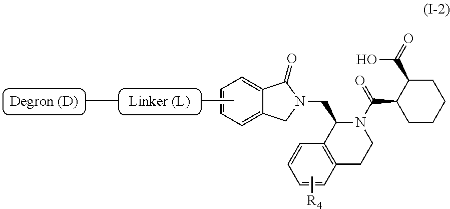
or a pharmaceutically acceptable salt or stereoisomer thereof,
wherein:
R₄ represents optionally substituted C1-C3 alkyl, OMe, or halo.

7. (canceled)

8. The bifunctional compound of claim 1, wherein the bifunctional compound has a structure represented by: 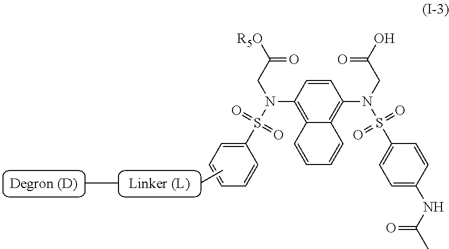
or a pharmaceutically acceptable salt or stereoisomer thereof,
wherein:
R₅ represents optionally substituted C1-C3 alkyl.

9. The bifunctional compound of claim 1, wherein the linker is an alkylene chain or a bivalent alkylene chain interrupted by, and/or terminating in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene, or any combination thereof, wherein R′ is H or C₁-C₆ alkyl, wherein the interrupting group and the terminating functional groups may be the same or different.

10. The bifunctional compound of claim 9, wherein the linker is

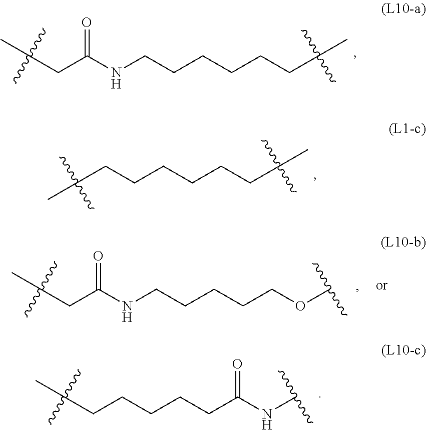 [SMILES: *CCCCCCNC(=O)C* ; *CCCCCC* ; *CC(=O)NCCCCCO* ; *CCCCCC(=O)N*]

11. (canceled)

12. The bifunctional compound of claim 1, wherein the Degron binds the E3 ubiquitin ligase which is cereblon, wherein the Degron is represented by the formula D1 or D2: 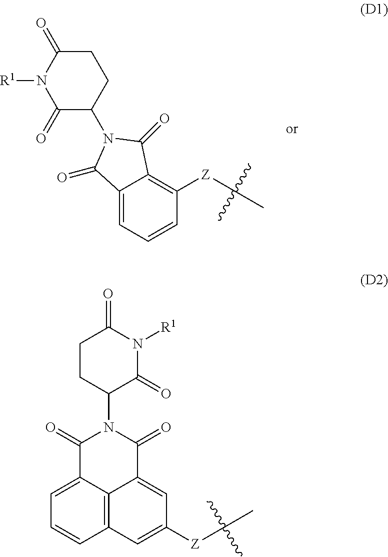
wherein
R¹ is H:
Z is NH, O, or C≡.

13. (canceled)

14. (canceled)

15. The bifunctional compound of claim 1, wherein the degron binds the E3 ubiquitin ligase which is von Hippel-Landau tumor suppressor, wherein the degron is represented by any of structures D3-D6: 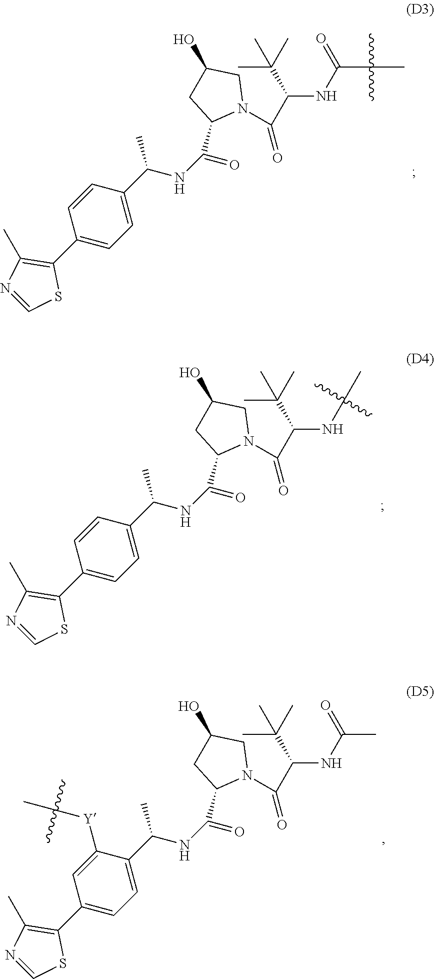
wherein Y′ is a bond, NH, O or CH₂; and 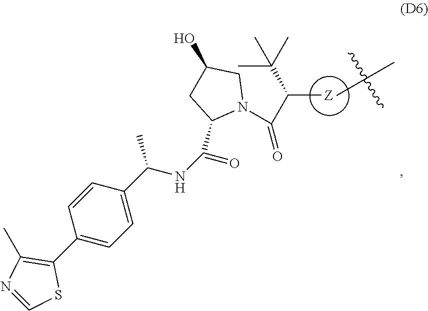
wherein Z is a cyclic group.

16. (canceled)

17. (canceled)

18. The bifunctional compound of claim 1, which is represented by any one of the following formulas: 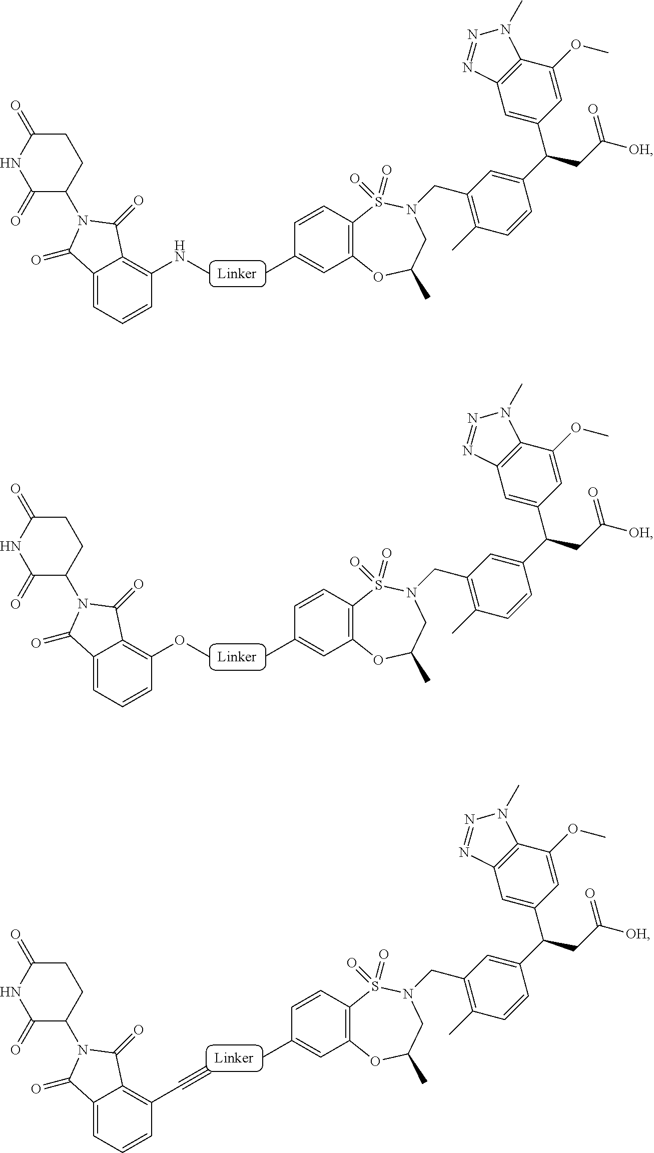 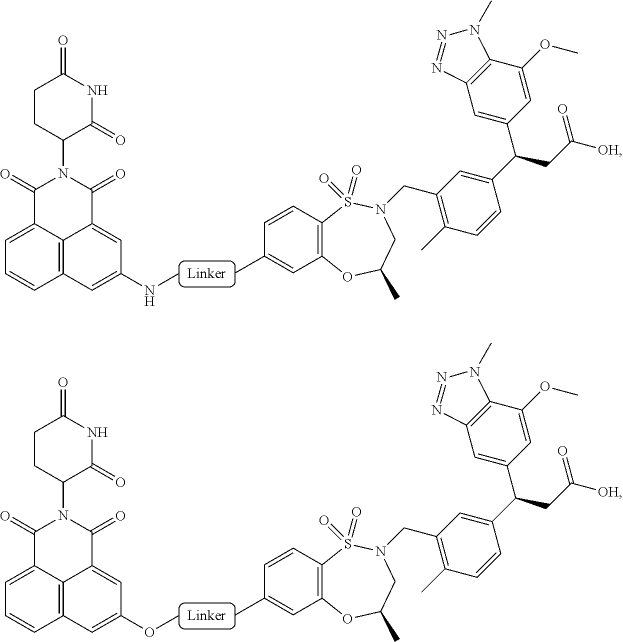 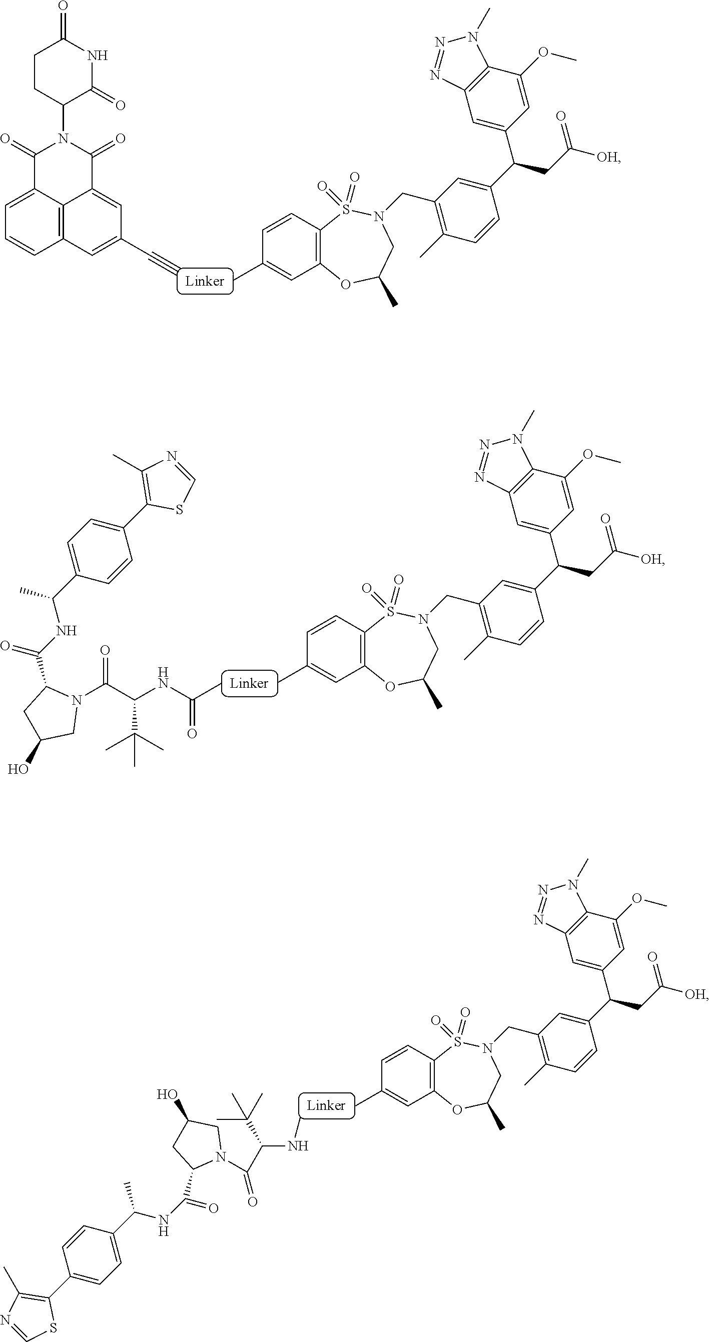 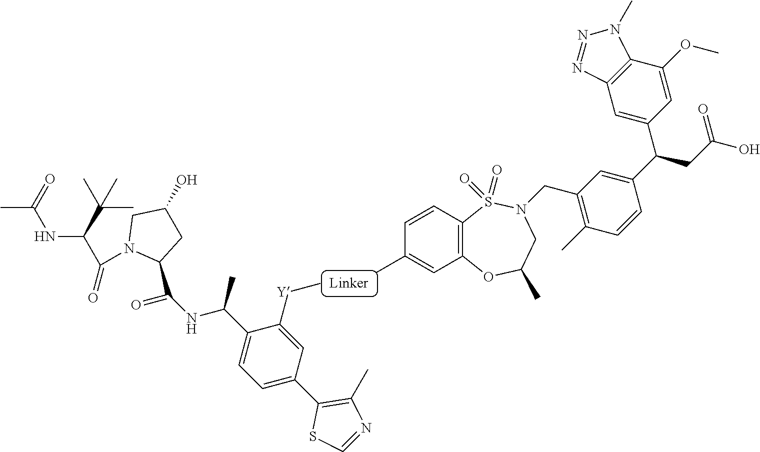
wherein Y′ is a bond, N, O or C, and 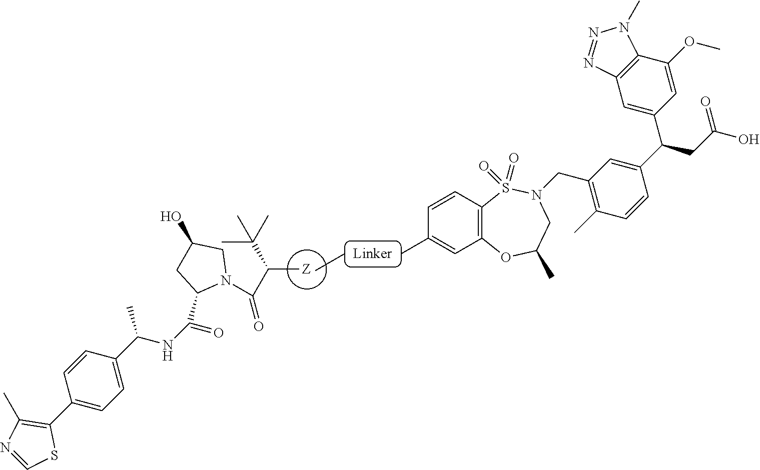
wherein Z is a cyclic group, or a pharmaceutically acceptable salt or stereoisomer thereof.

19. The bifunctional compound of claim 1, which is: 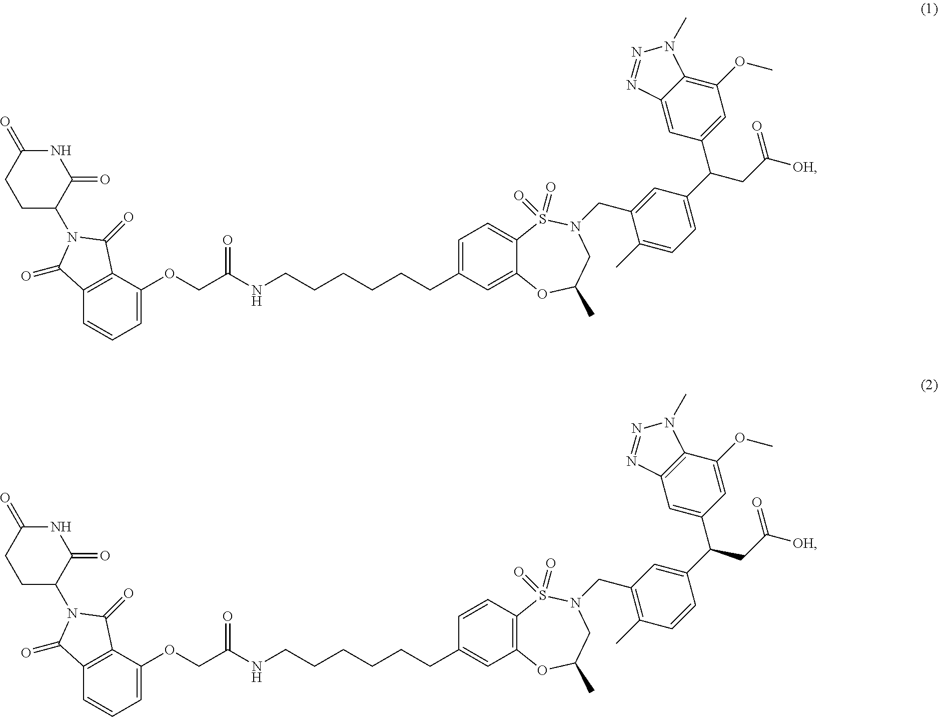 [SMILES: COc1cc(C(CC(=O)O)c2ccc(C)c(CN3C[C@@H](C)Oc4cc(CCCCCCNC(=O)COc5cccc6c5C(=O)N(C5CCC(=O)NC5=O)C6=O)ccc4S3(=O)=O)c2)cc2nnn(C)c12 ; COc1cc([C@@H](CC(=O)O)c2ccc(C)c(CN3C[C@@H](C)Oc4cc(CCCCCCNC(=O)COc5cccc6c5C(=O)N(C5CCC(=O)NC5=O)C6=O)ccc4S3(=O)=O)c2)cc2nnn(C)c12] 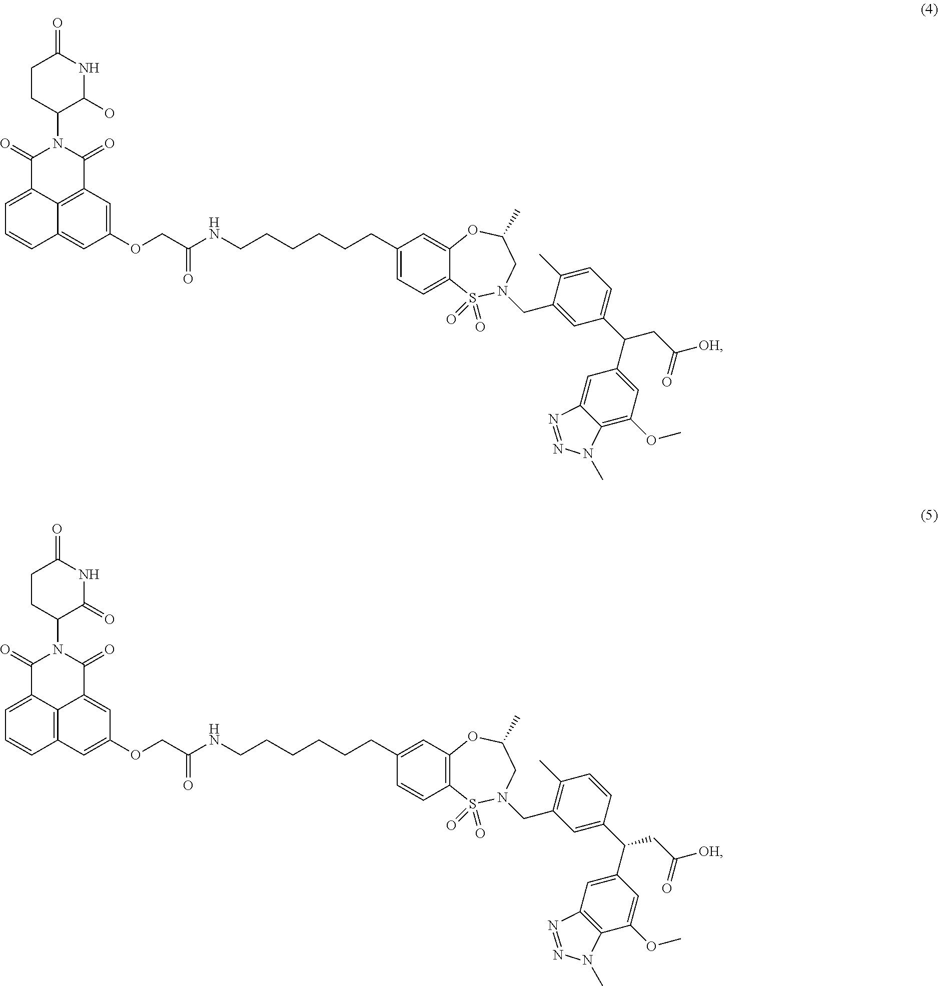 [SMILES: COc1cc(C(CC(=O)O)c2ccc(C)c(CN3C[C@@H](C)Oc4cc(CCCCCCNC(=O)COc5cc6c7c(cccc7c5)C(=O)N(C5CCC(=O)NC5O)C6=O)ccc4S3(=O)=O)c2)cc2nnn(C)c12 ; COc1cc([C@@H](CC(=O)O)c2ccc(C)c(CN3C[C@@H](C)Oc4cc(CCCCCCNC(=O)COc5cc6c7c(cccc7c5)C(=O)N(C5CCC(=O)NC5=O)C6=O)ccc4S3(=O)=O)c2)cc2nnn(C)c12] 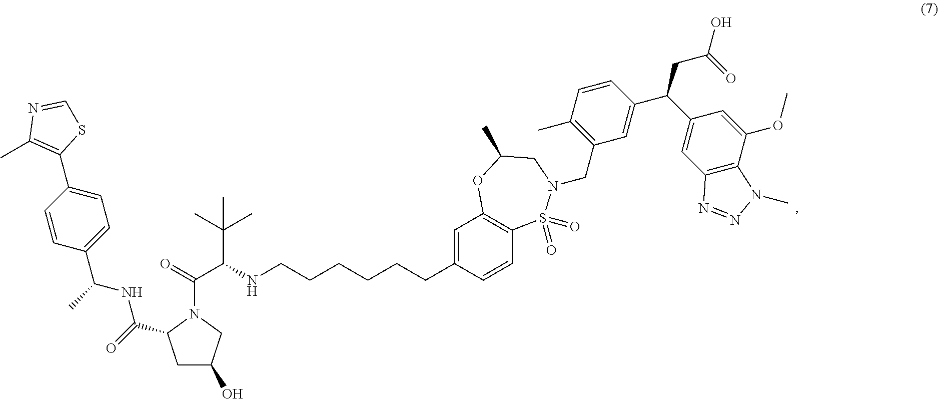 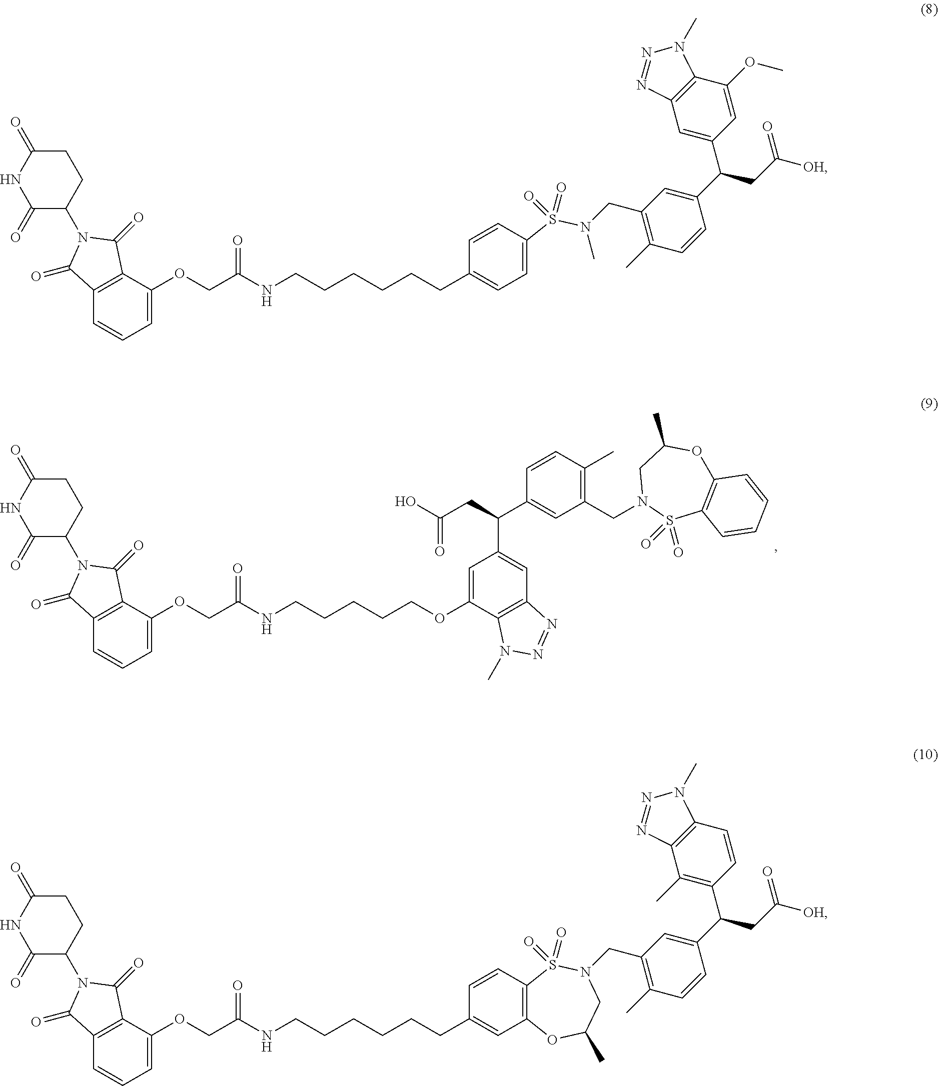 [SMILES: COc1cc([C@@H](CC(=O)O)c2ccc(C)c(CN(C)S(=O)(=O)c3ccc(CCCCCCNC(=O)COc4cccc5c4C(=O)N(C4CCC(=O)NC4=O)C5=O)cc3)c2)cc2nnn(C)c12 ; Cc1ccc([C@H](CC(=O)O)c2cc(OCCCCCNC(=O)COc3cccc4c3C(=O)N(C3CCC(=O)NC3=O)C4=O)c3c(c2)nnn3C)cc1CN1C[C@@H](C)Oc2ccccc2S1(=O)=O ; Cc1ccc([C@H](CC(=O)O)c2ccc3c(nnn3C)c2C)cc1CN1C[C@@H](C)Oc2cc(CCCCCCNC(=O)COc3cccc4c3C(=O)N(C3CCC(=O)NC3=O)C4=O)ccc2S1(=O)=O] 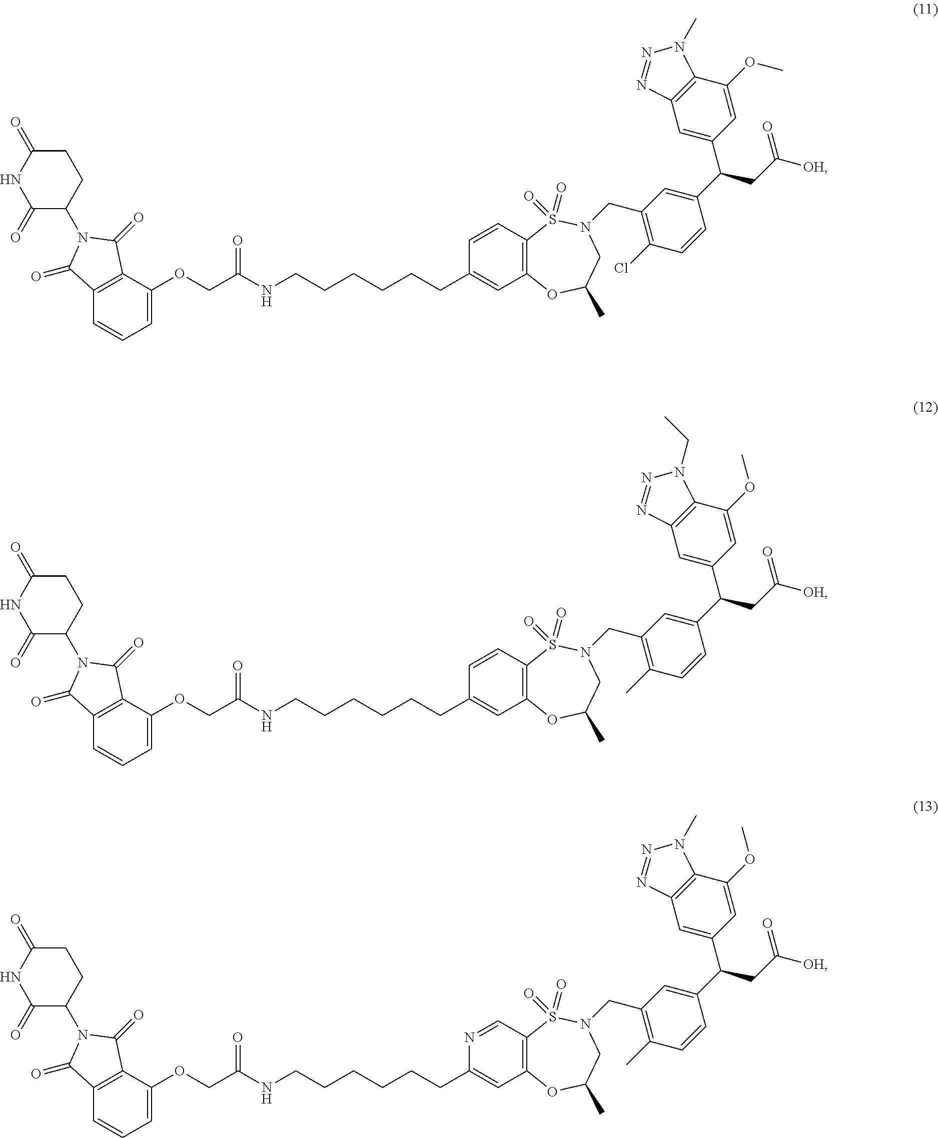 [SMILES: COc1cc([C@@H](CC(=O)O)c2ccc(Cl)c(CN3C[C@@H](C)Oc4cc(CCCCCCNC(=O)COc5cccc6c5C(=O)N(C5CCC(=O)NC5=O)C6=O)ccc4S3(=O)=O)c2)cc2nnn(C)c12 ; CCn1nnc2cc([C@@H](CC(=O)O)c3ccc(C)c(CN4C[C@@H](C)Oc5cc(CCCCCCNC(=O)COc6cccc7c6C(=O)N(C6CCC(=O)NC6=O)C7=O)ccc5S4(=O)=O)c3)cc(OC)c21 ; COc1cc([C@@H](CC(=O)O)c2ccc(C)c(CN3C[C@@H](C)Oc4cc(CCCCCCNC(=O)COc5cccc6c5C(=O)N(C5CCC(=O)NC5=O)C6=O)ncc4S3(=O)=O)c2)cc2nnn(C)c12] 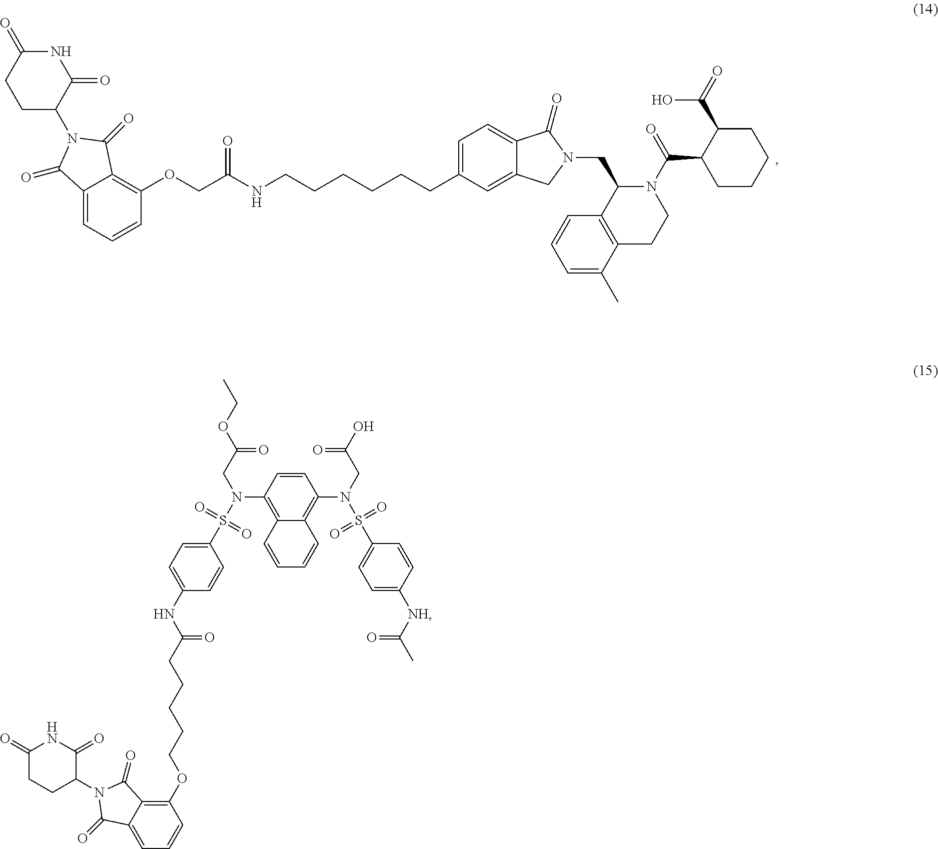 [SMILES: Cc1cccc2c1CCN(C(=O)[C@@H]1CCCC[C@@H]1C(=O)O)[C@@H]2CN1Cc2cc(CCCCCCNC(=O)COc3cccc4c3C(=O)N(C3CCC(=O)NC3=O)C4=O)ccc2C1=O ; CCOC(=O)CN(c1ccc(N(CC(=O)O)S(=O)(=O)c2ccc(NC(C)=O)cc2)c2ccccc12)S(=O)(=O)c1ccc(NC(=O)CCCCCOc2cccc3c2C(=O)N(C2CCC(=O)NC2=O)C3=O)cc1]
or a pharmaceutically acceptable salt or stereoisomer thereof.

20. A pharmaceutical composition, comprising a therapeutically effective amount of the compound of claim 1, or pharmaceutically acceptable salt or stereoisomer thereof, and a pharmaceutically acceptable carrier.

21. The method of treating a disease or disorder that is characterized or mediated by dysfunctional activity of KEAP1, comprising administering to a subject in need thereof a therapeutically effective amount of the compound of claim 1, or a pharmaceutically acceptable salt or stereoisomer thereof.

22. The method of claim 21, wherein the disease or disorder is characterized or mediated by oxidative stress.

23. The method of claim 21, wherein the disease or disorder is cancer.

24. The method of claim 23, wherein the cancer is non-small-cell lung carcinoma, colorectal cancer, cholangiocarcinoma, or breast cancer.

25. The method of claim 21, wherein the disease or disorder is diabetes, chronic kidney disease, cardiovascular disease, chronic obstructive pulmonary disease, or a neurodegenerative disease.
